(19) United States
(12) Patent Application Publication
(10) Pub. No.: US 2018/0317831 A1
(43) Pub. Date: Nov. 8, 2018

(54) DIAGNOSTIC TOOL FOR ASSESSING NEURODEVELOPMENTAL CONDITIONS OR DISORDERS

(71) Applicant: Murdoch Childrens Research Institute, Parkville (AU)

(72) Inventors: Vicki Anne Anderson, Clifton Hill (AU); Miriam Helen Beauchamp, Montreal (CA); David Gordon Darby, Mannerim (AU); Julian Dooley, Cleveland Heights, OH (US); Lyn S. Turkstra, Middleton, WI (US); Francesco Muscara, Victoria (AU); Simone Hearps, Brunswick (AU); George Charalambous, South Melbourne (AU); Sarva Thurai, Thornbury (AU); Mohinder Jaimangal, Mount Pleasant (AU)

(21) Appl. No.: 16/038,064

(22) Filed: Jul. 17, 2018

Related U.S. Application Data
(63) Continuation of application No. PCT/AU2016/050023, filed on Jan. 19, 2016.

(51) Int. Cl. A61B 5/16; A61B 5/00; G16H 10/20; G16H 50/20; G09B 7/00

(52) U.S. Cl. CPC A61B 5/167; A61B 5/168; A61B 5/7445; A61B 2560/0475; G16H 10/20; G16H 50/20; G09B 7/00; A61B 5/002

(57) ABSTRACT

A diagnostic tool for assessing symptoms of neurodevelopmental conditions or disorders of patients and providing an objective measurement of symptoms of such conditions or disorders, including deficiencies in social competency of a patient. The tool can include a health data collection interface enabling a patent to interact with the tool and provide feedback to stimulus provided by the diagnostic tool in in each of at least three test areas related to the patient's ability to intuit emotional state from physical cues. The tool can further include an automated health data assessment device configured to obtain the social feedback of the patient for each test area of the at least three test areas, and to automatically assess social competency of the patient based on the social feedback and to generate an objective measurement of the social competency of the patient for use in diagnosing the patient for a potential neurodevelopmental condition.

### TECHNICAL FIELD

Described embodiments relate to devices for diagnosing and assessing health conditions, and specifically to diagnosing and assessing neurodevelopmental conditions or disorders.

### BACKGROUND

Over recent decades, the medical community has increasingly become aware of the seriousness and pervasiveness of neurodevelopmental conditions or disorders, such as autism spectrum disorders. At the same time, other neurological conditions are also gaining recognition, such as attention deficit hyperactivity disorder, depression, language and learning difficulties, epilepsy. These conditions or disorders present with a variety of symptoms, including deficiencies in social development.

A well-recognized problem in diagnosing neurodevelopmental conditions or disorders is the difficulty in accurately and objectively assessing symptoms of patients. Because these symptoms often relate to social interactions, such as an inability for the patient to process physical cues of others and intuit their emotional state, diagnostic and assessment mechanisms often rely on a subjective human assessment. The subjective nature of this type of assessment, in turn, leads to wide disparities in how and how often patients are diagnostic with a neurodevelopmental condition. Moreover, human assessors frequently harbor implicit biases that inhibit accurate assessment. Certain communities or individuals may, for example, believe that neurodevelopmental conditions are simply a personality trait or choice, and thus not require medical intervention. Because social problems have also been established as comorbidities or precipitants for later mental health problems, delinquency and criminal behaviour, the subjective nature of diagnostic methods can lead to real and substantial consequences to patients and others.

Thus, what is needed is an accurate, objective tool for diagnosing neurodevelopmental conditions or disorders.

### BRIEF DESCRIPTION OF DRAWINGS

Embodiments are described in further detail below, by way of example and with reference to the accompanying drawings, in which:

FIG. 1 is a block diagram of an example of an assessment system for assessing social competency of a user;

FIG. 2 is a process flow diagram of a computer-implemented method of assessing social competency according to some embodiments, the method implementable by the assessment system of FIG. 1;

FIG. 3 is a block diagram of an example of a communications system comprising an assessment system comprising a server and a computing device in communication with the server across a communications network, according to some embodiments;

FIG. 4 is a process flow diagram of a computer-implemented method for assessing social competency according to some embodiments, the method operable by the server of FIG. 3; and

FIG. 5 is a process flow diagram of a computer-implemented method of assessing social competency according to some embodiments, the method operable by one of the computing devices of FIG. 3.

FIG. 6 is an example screen shot of an assessment platform displayed on a user interface of the assessment system of FIG. 1 or FIG. 3;

FIG. 7 is a process flow diagram depicting a computer-implemented method of determining a measure of selective attention of a user of the assessment system of FIG. 1 or FIG. 3, according to some embodiments;

FIG. 8 is an example screen layout of a game to determine a measure of selective attention displayed on a user interface of the assessment system of FIG. 1 or FIG. 3;

FIG. 9 is an example screen shot of a game to determine a measure of selective attention displayed on a user interface of the assessment system of FIG. 1 or FIG. 3;

FIG. 10 is a process flow diagram depicting a computer-implemented method of determining a measure of reaction time of a user of the assessment system of FIG. 1 or FIG. 3, according to some embodiments,

FIG. 11 is an example screen layout of a game to determine a measure of reaction time displayed on a user interface of the assessment system of FIG. 1 or FIG. 3;

FIG. 12 is an example screen shot of a game to determine a measure of reaction time displayed on a user interface of the assessment system of FIG. 1 or FIG. 3;

FIG. 13 is a process flow diagram depicting a computer-implemented method of determining a measure of speed and/or information processing of a user of the assessment system of FIG. 1 or FIG. 3, according to some embodiments.

FIG. 14 is an example screen layout of a game to determine a measure of speed and/or information processing displayed on a user interface of the assessment system of FIG. 1 or FIG. 3;

FIG. 15 is an example screen shot of a game to determine a measure of speed and/or information processing displayed on a user interface of the assessment system of FIG. 1 or FIG. 3;

FIG. 16 is a process flow diagram depicting a computer-implemented method of determining a measure of non-verbal language comprehension of a user of the assessment system of FIG. 1 or FIG. 3, according to some embodiments,

FIG. 17 is an example screen layout of a game to determine a measure of non-verbal language comprehension displayed on a user interface of the assessment system of FIG. 1 or FIG. 3;

FIG. 18 is an example screen shot of a game to determine a measure of non-verbal language comprehension displayed on a user interface of the assessment system of FIG. 1 or FIG. 3;

FIG. 19 is a process flow diagram depicting a computer-implemented method of determining a measure of a user's ability to comprehend social intent of a user of the assessment system of FIG. 1 or FIG. 3, according to some embodiments,

FIG. 20 is an example screen layout of a game to determine a measure of a user's ability to comprehend social intent displayed on a user interface of the assessment system of FIG. 1 or FIG. 3;

FIG. 21 is an example screen shot of a game to determine a measure of a user's ability to comprehend social intent displayed on a user interface of the assessment system of FIG. 1 or FIG. 3;

FIG. 22 is a process flow diagram depicting a computer-implemented method of determining a measure of pragmatics of a user of the assessment system of FIG. 1 or FIG. 3, according to some embodiments,

FIG. 23 is an example screen layout of a game to determine a measure of pragmatics of a user displayed on a user interface of the assessment system of FIG. 1 or FIG. 3;

FIG. 24 is an example screen shot of a game to determine a measure of pragmatics of a user displayed on a user interface of the assessment system of FIG. 1 or FIG. 3;

FIG. 25 is a process flow diagram depicting a computer-implemented method of determining a measure of prosody of a user of the assessment system of FIG. 1 or FIG. 3, according to some embodiments,

FIG. 26 is an example screen layout of a game to determine a measure of prosody of a user displayed on a user interface of the assessment system of FIG. 1 or FIG. 3;

FIG. 27 is a process flow diagram depicting a computer-implemented method of determining a measure of ability of a user of the assessment system of FIG. 1 or FIG. 3 to recognise emotion, according to some embodiments,

FIG. 28 is an example screen layout of a game to determine a measure of ability of a user to recognise emotion displayed on a user interface of the assessment system of FIG. 1 or FIG. 3;

FIG. 29 is an example screen shot of a game to determine a measure of ability of a user to recognise emotion displayed on a user interface of the assessment system of FIG. 1 or FIG. 3;

FIG. 30 is a process flow diagram depicting a computer-implemented method of determining a measure of emotion perception of self and others of a user of the assessment system of FIG. 1 or FIG. 3, according to some embodiments

FIG. 31 is an example screen layout of a game to determine a measure of emotion perception of self and others displayed on a user interface of the assessment system of FIG. 1 or FIG. 3;

FIG. 32 is an example screen shot of a game to determine a measure of emotion perception of self and others displayed on a user interface of the assessment system of FIG. 1 or FIG. 3;

FIG. 33 is a process flow diagram depicting a computer-implemented method of determining a measure of attribution of social intent and/or social information processing of a user of the assessment system of FIG. 1 or FIG. 3, according to some embodiments,

FIG. 34 is a process flow diagram depicting a computer-implemented method of determining a measure of socio-moral solutions, perspective taking/empathy, and/or moral reasoning of a user of the assessment system of FIG. 1 or FIG. 3, according to some embodiments;

FIG. 35 is an example screen layout of a game to determine a measure of socio-moral solutions, perspective taking/empathy, and/or moral reasoning displayed on a user interface of the assessment system of FIG. 1 or FIG. 3;

FIG. 36 is an example screen shot of a game to determine a measure of socio-moral solutions, perspective taking/empathy, and/or moral reasoning displayed on a user interface of the assessment system of FIG. 1 or FIG. 3;

FIG. 37 is an example screen shot of a game to determine a measure of socio-moral solutions, perspective taking/empathy, and/or moral reasoning displayed on a user interface of the assessment system of FIG. 1 or FIG. 3;

FIG. 38 is an example screen shot of a game to determine a measure of socio-moral solutions, perspective taking/empathy, and/or moral reasoning displayed on a user interface of the assessment system of FIG. 1 or FIG. 3;

FIG. 39 is a process flow diagram depicting a computer-implemented method of determining a measure of theory of mind of a user of the assessment system of FIG. 1 or FIG. 3, according to some embodiments;

FIG. 40 is a process flow diagram depicting a computer-implemented method of determining a measure of theory of mind of a user of the assessment system of FIG. 1 or FIG. 3, according to some embodiments;

FIG. 41 is an example screen layout of a game to determine a measure of theory of mind displayed on a user interface of the assessment system of FIG. 1 or FIG. 3;

FIG. 42 is an example screen shot of a game to determine a measure of theory of mind displayed on a user interface of the assessment system of FIG. 1 or FIG. 3;

FIG. 43 is a process flow diagram depicting a computer-implemented method of administering a computer-based social skills questionnaire, according to some embodiments; and

FIG. 44 is an example screen layout of the computer-based social skills questionnaire displayed on a user interface of the assessment system of FIG. 1 or FIG. 3.

### DESCRIPTION OF EMBODIMENTS

Described embodiments relate to diagnostic tools for assessing symptoms of neurodevelopmental conditions or disorders, including systems and computer-implemented methods for assessing social competency. In particular, described are assessment systems, assessment servers and computer-implemented methods for assessing social competency of a user.

Some embodiments relate to an assessment application or tool for facilitating the assessment of social competency of a user of the assessment system. The assessment application is deployed on or implemented by the assessment system and when executed by one or more processors of the assessment system, is configured to provide an assessment platform to the user, for example, by displaying the assessment platform on a user interface of the assessment system, to facilitate the social competency assessment.

In some embodiments, the assessment system is a standalone computing device, such as a table or laptop. In other embodiments, the assessment system is a distributed system comprising a server and a computing device in communication with the server across a communications network. In some embodiments, the assessment application is deployed on or implemented by the server and is configured to cooperate with a browser application deployed on the computing device to provide the assessment platform to a user interface of the computing device to allow a user to engage or interact with the assessment system and to assess the social competency of the user. In other embodiments, the assessment application is distributed between the server and the computing device, with one or more code modules of the assessment application being deployed on the server and one or more code modules of the assessment application being deployed on the computing device and the server and computing device cooperating with one another to provide the assessment platform to a user interface of the computing device to allow a user to engage or interact with the assessment system and to assess the social competency of the user.

The assessment application includes a games or testing module and an assessment module. The games module includes at least one interactive game component in each of at least three social domains to test a patient's social functions across the social domains. For example, the at least three social domains comprise attention/executive function, communication, and social cognition. Each of the interactive game components are configured to provide an interactive game to the assessment platform and to determine a result or an outcome for the interactive game based on user inputs from the user at the computing device. For example, the outcome may comprise data relating to or based on the user inputs provided by the user in response to prompts from the game component when the user is interacting with the game, and which may be determined by monitoring by the games module of the user's interaction with the game.

The assessment module is configured to assess social competency of the user of the assessment platform based on the outcome of at least one interactive game in each of the at least three social domains and to generate an assessment outcome. The assessment module may also employ data other than the outcomes of the interactive games in determining the assessment outcome. For example, the assessment module may employ data stored in a user record associated with the user in determining the assessment. In some embodiments, the assessment module may determine a social quotient or “social IQ score” for the user indicative of social competency of the user and which may be employed to guide professional interpretation of the user's social competency and identification of appropriate early intervention measures. For example, in some embodiments, the assessment module is configured to determine the social quotient by referencing a collection or pool or normative data. Thus, the assessment module may be configured to determine a measure of social competency of the user. In some embodiments, the assessment application may be further configured to diagnose the social competency of the user based on data determined by the assessment module.

The described systems and computer implemented methods realize the genuine benefits of digital health by converging health and technological advances in information technology software and hardware (tablet devices, touch screens), thereby providing professionals with convenient, low cost, easy to interpret assessment tools which deliver an automated, comprehensive ‘social profile’ and introduce efficiencies and cost-saving for the healthcare system through real-time, automated data capture, analysis and scoring.

The described systems and computer implemented methods may be considered as providing a digital social assessment application or tool for specific groups of users which may allow social strengths and weaknesses of a user or patient to be profiled and/or categorised to diagnose social impairment and thereby assisting health and education professionals to intervene with tailored or targeted treatment or assistance for social disorders. For example, the computerised assessment tool may be used to assess, identify and/or diagnose social impairments or problems in children, adolescents, and adults. It may be employed in a variety of sectors such as child health, mental health, education, employment, recruitment and aged care. For example, the computerised assessment tool may be used as a screening tool for schools, adult psychological testing market, and/or recruitment industry in which assessment of social skills are a key priority.

The digital social assessment application or tool may be ‘ecologically sensitive’, creating a virtual environment or situation that closely simulates real life experience and allows the user to become a part of the social situation. For example, the virtual environment may simulate the real-world by using real life stimuli (child faces and familiar situations), and may thereby optimize the likelihood of emotional engagement, motivation and participation.

The described systems and computer implemented methods may provide for a more accurate assessment of social competency by taking the user's level and motivation into account, for example, by providing an assessment platform that is action orientated, fun and allows for the accurate capture of objective data direct from the user in real-time. In particular, by allowing for the data. i.e., the outcome of the games, to be gathered or sampled from the individual user directly in an objective as opposed to subjective manner (i.e., without reliance on the tester's perspective), the gathered data is untainted or affected by a bias of the tester and accordingly, the assessment of the social competency of the user may be more accurate.

The described systems and computer implemented methods may provide for a more simple and user-friendly assessment of social competency. For example, current assessments require the patient to interact with the professional in a meaningful way in a formal setting, which may therefore have limited ecological validity. The described systems and computer implemented methods are likely to be more attractive to patients as they allow the patient to be tested in a suitable, yet relatively stress-free, environment.

The described systems and computer implemented methods add an objective, repeatable and comprehensive measure in assessing the social competency of the user, providing the ability to quantify social skills and extend on current reliance on third party rating measures such as parent and teacher rating measures, to patient-direct methods.

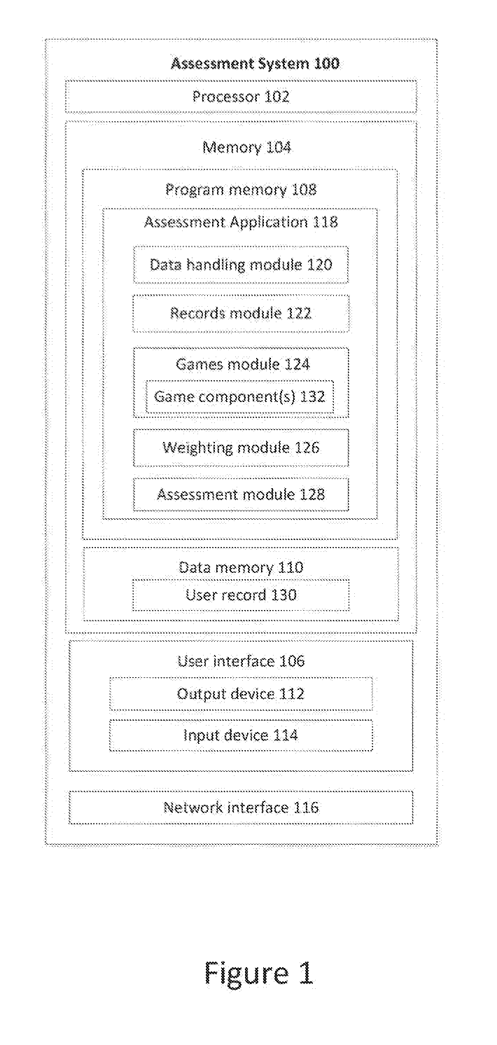

Referring to FIG. 1, there is illustrated an example of an assessment system 100, according to some embodiments. The assessment system 100 comprises at least one processor 102, one or more forms of memory 104, and a user interface 106. The one or more processors 102 (referred to for ease of reference as “processor 102”) includes components such as digital signal processing units (DSPUs), central processing units (CPUs), arithmetic logic units (ALUs) and/or registers for storing data.

Memory 104 comprises a combination of volatile (e.g. RAM) and non-volatile e.g. hard disk drive, solid state drive, flash memory and/or optical disc) computer readable storage. In particular, memory 104 comprises program memory 108 and data memory 110. For example, program memory 108 may include read-only memory (ROM) such as erasable ROM (EROM) and electrically erasable programmable ROM (EEPROM or flash ROM), or random access memory (RAM) such as static RAM (SRAM), dynamic RAM (DRAM) or non-volatile RAM (NVRAM or flash). Program memory 108 has sufficient capacity to store program code executable by the processor 102 in order to perform appropriate processing functions as described herein. Data memory 110 may be configured to store data pertinent to operations of the assessment system 100 and/or to store information pertaining to the assessment platform.

The user interface 106 comprises at least one output device 112, such as a display and/or speaker, for providing an output to the user of the assessment system 100 and at least one input device 114, such as a touch-screen, a keyboard, mouse, microphone, video camera, stylus, push button, switch or other peripheral device that can be used for providing user input to the assessment system 100.

In some embodiments, the assessment system 100 may further comprise a network interface 116. The network interface 116 comprises suitable hardware and software interfaces to facilitate wireless and/or wired communication with a remote server (not shown) and/or other computing devices, systems or servers. For example, the network interface 116 may comprise a USB port, Ethernet port, a wireless adapter or a Bluetooth module.

As shown in FIG. 1, program memory 108 comprises an assessment application 118 for assessing social competency. The assessment application 118 comprises program code, such as executable code modules, which, when aggregated and executed by the processor 102, performs programmed functions as described herein. In some embodiments, such code modules may include a data handling module 120, a records module 122, games module 124, a weightings module 126, and an assessment module 128.

The data handling module 120, when executed by the processor 102, is configured to pass data, such as requests and responses, between the user interface 106 and the relevant module(s) of the assessment application 118.

The records module 122, when executed by the processor 102, is configured to create, store, modify and delete user records 130 associated with users of the assessment platform. For example, each user record 130 may comprise personal details of the user, such as name, age, contact information, information relating to social competency of the user, outcomes of games played in the assessment platform by the user, and/or determinations made by the assessment module 118, etc. In some embodiments, the user records 130 are stored locally in data memory 112. However, it will be appreciated that the user records 130 may be stored elsewhere, for example in a remotely located database (not shown), on an external storage device (not shown), in addition or as an alternative to the local storage.

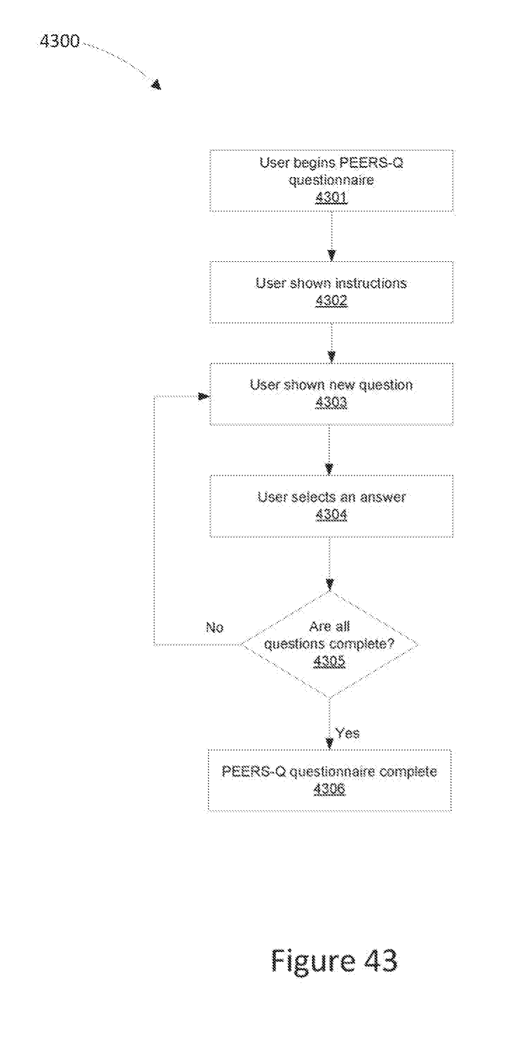

In some embodiments, information determined from responses to one or more social skills questionnaires, such as a computer-based social skills questionnaire may be associated with the user and stored in their user record and may be employed in assessing the user social competency. For example, such social skills questionnaires may be provided to those associated with the user, such as friends, family, teachers, employers, colleagues, supervisors and/or the patient or user themselves. The social skills questionnaires may be designed to determine and provide a rating for mediators of social function, such as developmental stage, brain integrity, personal attributes, environment, and social outcomes, such as social participation and mental health. In some embodiments, the social skills questionnaires may be designed to determine an assessment of one or more of (i) externalizing behaviours vs. internalizing behaviours, (ii) psychological problems depression and/or anxiety, (iii) Participation loneliness, withdrawal, and/or home/community/school participation, (iv) personality/temperament and/or shyness vs. extraversion, and (v) physical appearance height, weight, disabilities. The social skills questionnaires may also provide a number of general questions regarding numbers of friends, regular activities, and/or social opportunities and employ the answers to these questions in determining the assessment of the above factors. An example of a method of administering a computer-based social skills questionnaire is discussed below with reference to FIG. 43.

In some embodiments, data gathered from users of the assessment platform and/or from the social skills questionnaires is categorised and standardized to create age and gender norms for the diagnosis of social impairment and this normative data may be stored at a remote database and may be accessible to the assessment system 100 via the network interface 116. In some embodiments, the normative data may be stored locally in the data memory 110 of the assessment system 100.

In some embodiments, normative data may be collected by conducting a standardisation project of the assessment system 100, which may involve assessing a population-based sample using the assessment system 100. For example, the normative data may be based on regression-based continuous norming to result in minimal associated uncertainty. In some cases, data may be factor analysed, using exploratory and confirmatory methods, to check statistical validity of theoretical domains. When data supports it, domains may be adjusted to meet findings.

The games module 124, when executed by the processor 102, may be configured to provide a plurality of interactive games or tests to the assessment platform displayed on the output device 112 of the user interface 106. The games module 124 may comprise a plurality of interactive game components 132, and each interactive game component, when executed by the processor 102, may be configured to provide an interactive game to the assessment platform to allow the user of the assessment system 100 to play the game and to enable the games module 124 to determine an outcome of the game played by the user. The outcome of the game may provide an effective indication or assessment of a specific skill or ability of the user. For example, the outcome may comprise data relating to or based on the user inputs provided by the user in response to prompts from the game component 132 when the user is interacting with the game, and which may be determined by monitoring by the games module of the user's interaction with the game.

The interactive games may be categorised according to different types of games, each type associated with a social domain. For example, the social domains may comprise attention/executive function, communication, and social cognition. The attention/executive function game(s) may be configured to determine a measure of attention/executive function of a user playing the attention/executive function game(s), the communication game(s) may be configured to determine a measure of communications ability of a user playing communication game(s), and the social cognition game(s) may be configured to determine a measure of social cognition ability of a user playing the social cognition game(s).

In some embodiments, the interactive games of each type may include basic versions of the interactive game and more complex version of the interactive game. In some embodiments, the interactive games, whether the basic or more complex version, may include an introductory level and an assessment level. For example, the introductory level may be provided to allow the user to try the interactive game to ensure they are comfortable with how it works and what they are required to do to effectively play the game before progressing to the assessment level. In some embodiments, the outcome of the introductory level of the game for a user is not employed by the assessment module 128 in assessing a social competency of the user.

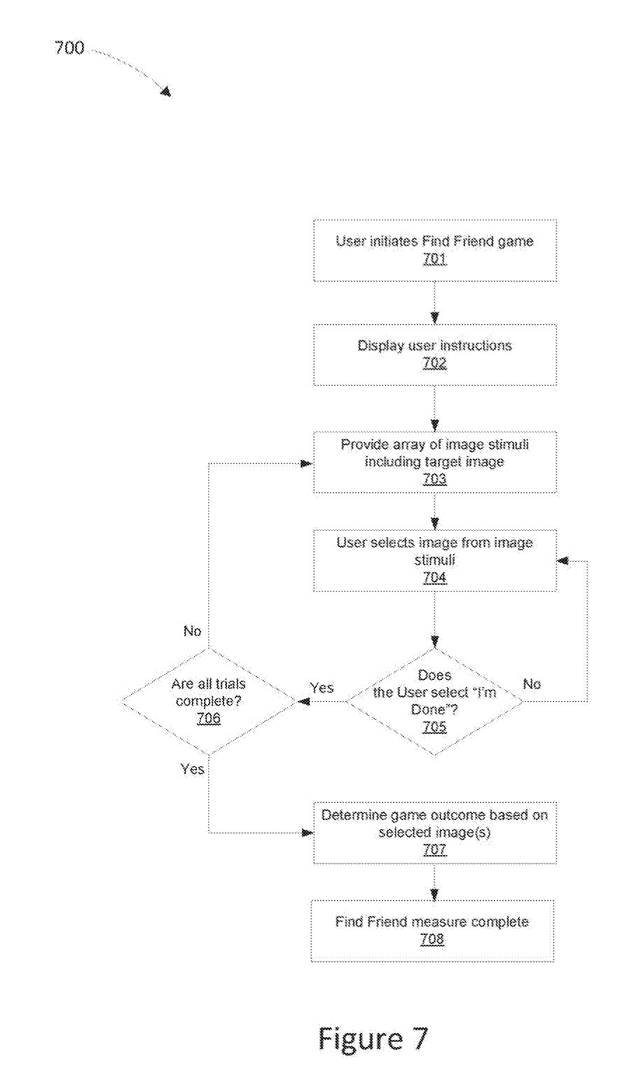

Examples of a plurality of social domain types of interactive games delivered by the interactive game component(s) 132 of the games module 124 are discussed in more detail below with reference to FIGS. 7 to 42.

The weighting module 126, when executed by the processor 102, is configured to apply weightings to outcomes of interactive games played by users of the assessment platform. For example, interactive games provided on the assessment platform may be accorded different levels of importance in assessing the social competency of a user engaging in the assessment platform. Accordingly, the outcomes of the interactive games played by a user may be weighted before the assessment module 128 determines an assessment of the social competency of the user based on the outcomes of the interactive games played by the user on the assessment platform.

The assessment module 128, when executed by the processor 102, may be configured to determine an assessment of the social competency of a user engaging with the assessment platform provided by the assessment system 100. The assessment module 128 may base the assessment on the outcomes of at least some of the interactive games played by the user, and in some embodiments, the weighted outcomes of the at least some of the interactive games played by the user. In some embodiments, the assessment module 128 may base the assessment on the outcome of at least one interactive game associated with each of the social domains of attention/executive function, communication, and social cognition. The assessment module 128 may also employ data other than the outcomes of the interactive games in determining the assessment. For example, the assessment module 128 may employ data stored in the user record 130 associated with the user in determining the assessment and/or the normative data as discussed above.

In some embodiments, the assessment module 128 may determine a social quotient or “social IQ score” for the user indicative of social competency of the user and which may be employed to guide professional interpretation of the user's social competency and identification of appropriate early intervention measures.

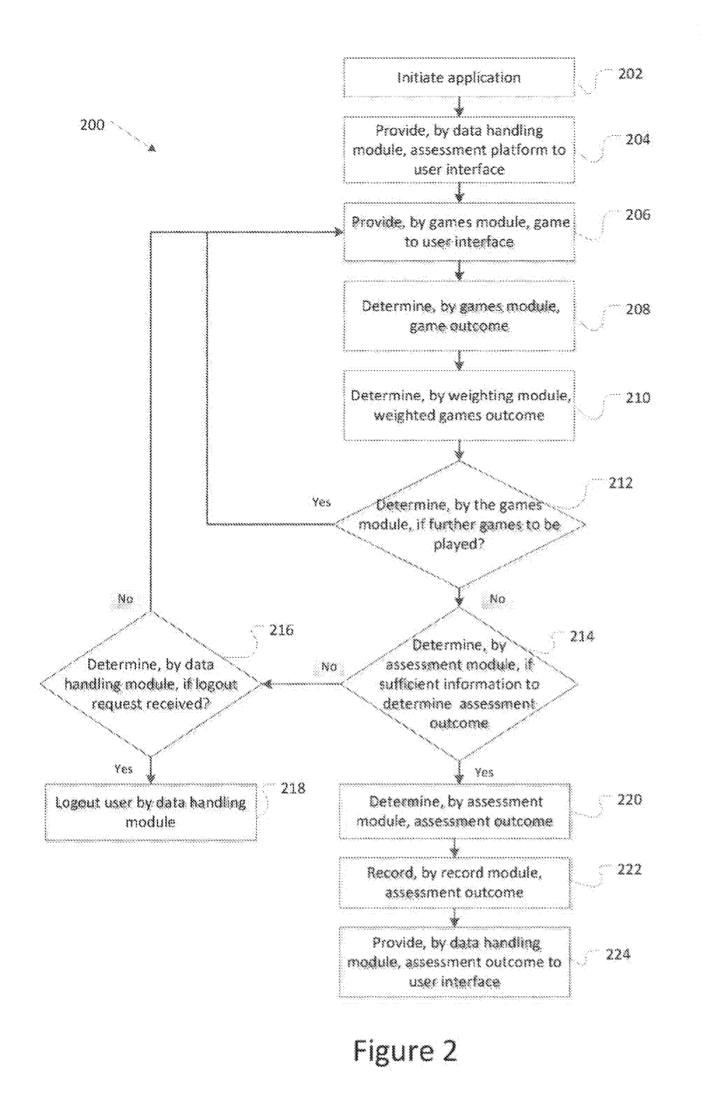

Referring now to FIG. 2, there is shown a process flow diagram of a computer-implemented method 200 of assessing social competency according to some embodiments, the method 200 operable by the assessment system 100. In some embodiments, the data handling module 120, the records module 122, the games module 124, and the assessment module 128, and in some embodiments, the weighting module 126, are executed by the processor 102 in order to perform the functions of the method 200 as described.

The method 200 begins with the initiation of the assessment application 118, at 202. For example, a user of the assessment system 100 can initiate the application by turning on the assessment system 100 and/or clicking on an icon shown on a display of the output device 112 of the user interface 106.

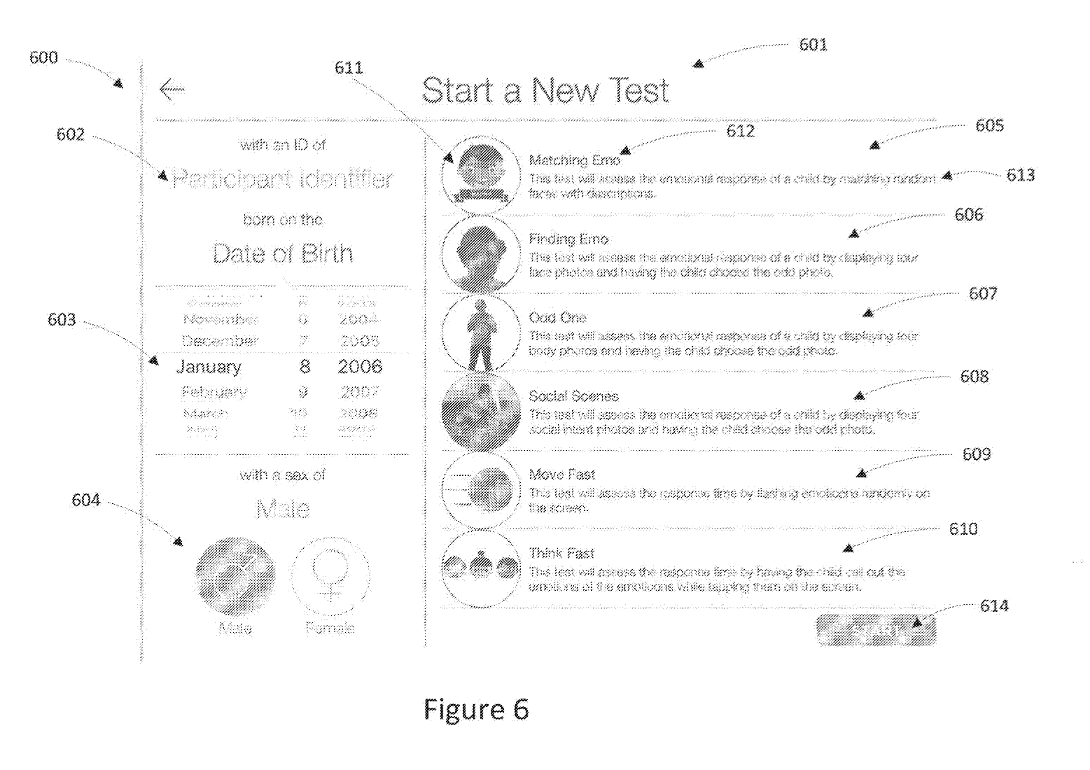

Once the assessment application 118 has been initiated, an assessment platform is provided to output device 112 of the user interface 106, by the data handling module 120 of the assessment application 118, at 204. For example, the assessment platform may be displayed on a display of the output device 112, as discussed in more detail below with reference to FIG. 6.

The games module 124 provides an interactive game to the assessment platform, at 206. In some embodiments, the data handling module 120 receives a game selection from the input device 114 and the games module 124 provides an interactive game corresponding to the game selection to the assessment platform in response to receiving the game selection. In some embodiments, games may be selected by the assessment application 118 for the user, for example, automatically in response to the initiation of the assessment application 118 or in response to receiving, by the data handling module 120 from the input device 114, an indication that automatic selection of the games by the assessment application 118 for the user is desired. In some embodiments, the game(s) selected by the games module 124 for the user is based on information received from the records module 122 about the user, for example, from the user record 130. In some embodiments, some games may be automatically selected by the assessment application 118 for the user and the user may be allowed to select other games from a plurality of user selectable games options provided on the assessment platform.

The games module 124 determines an outcome of the interactive game based on the user's inputs received via the input device 114 when the user is engaging or playing the interactive game, at 208. In some embodiments, the records module 122 records the game outcome, for example, in user records 130.

The manner in which the games module 124 provides the games to the assessment platform and monitors the user's interaction or engagement with the game to determine the game outcomes will depend on the game which has been played and is discussed in more detail below with reference to FIGS. 7 to 42.

In some embodiments, the games module 124 passes the game outcome to the weighting module 126 and the weighting module 126 determines a weighted game outcome based on the game outcome and a game weighting, at 210. Different games may be accorded different weightings based on their deemed importance or priority in determining an assessment of the user's social competency. For example, the game weighting for a given game may depend on the other games which are been used to determine the assessment, whether the game is a basic or complex version, to which social domain type the game belongs, and/or the characteristics of the game. The game weighting may be stored in locally in data memory 110 and/or for example, at a remote database (not shown). In some embodiments, where no weighting module 126 is employed, the games module 124 may pass the game outcome of the game directly to the assessment module 128.

The games module 124 determines whether further game(s) are to be played, at 212. If the games module 124 determines that a further game is to be played, for example, in response to receiving, by the data handling module 120, a further game selection from the input device 114 and/or in response to determining, by the games module 124, an automatically selected further interactive game, the games module 124 provides the game selection of the interactive game to the assessment platform, at 206.

If the games module 124 determines that a further game is not to be played, for example, in response to receiving, by the data handling module 120 from the input device 114, a request for an assessment outcome, and/or in response to determining, by the games module 124, that no further interactive games are to be provided, the assessment module 128 determines whether there is sufficient information to determine an assessment outcome, at 214. For example, in some embodiments, the assessment module 128 determines whether a game outcome has been determined for at least one interactive game in each of the at least three social domains, i.e., attention/executive function, communication, and social cognition.

In some embodiments, if the assessment module 128 determines that there is insufficient information to determine an assessment outcome, at 214, the data handling module 120 determines whether a logout or other session termination notification has been received from the input device 114, at 216. If the data handling module 206 determines that a logout or other session termination notification has been received from the input device 114, the data handling module 120 logs out the user and/or ends the session. In some embodiments, the records module 122 records the games outcomes determined during the session in the user record 130. If the data handling module 120 determines that a logout or other session termination notification has not been received from the input device 114 at 216, the assessment application 118 determines and provides a game selection to the assessment platform at 206. In other embodiments, if the assessment module 128 determines that there is insufficient information to determine an assessment outcome, at 214, the assessment application 118 determines and provides a game selection to the assessment platform at 206.

If the assessment module 128 determines that there is sufficient information to determine an assessment outcome, at 214, the assessment module 128 determines an assessment outcome based on the game outcomes and/or weighted game outcomes associated with the user, at 220. In some embodiments, the assessment module 128 may also base the assessment outcome on information in the user record 130 and/or the normative data. In some embodiments, the assessment outcome comprises a social competency value such as a social quotient or social IQ. For example, in some embodiments, the assessment module 128 compares each game outcome and/or weighted game outcome with the normative data for the gender and/or age of the user in order to determine the assessment outcome.

In some embodiments, the assessment module 128 may cause the data handling module 120 to retrieve the normative data from a remote server (not shown) via the network interface 116. In some embodiments, the assessment module 128 may cause the data handling module 120 to transmit information associated with the user, including the outcome of the at least one interactive game, to a remote server (not shown) via the network interface 116 and to receive an assessment of the social competency of the user from the remote server (not shown) via the network interface 116 and to pass the assessment of the social competency of the user to the assessment module 128. The assessment module 128 may then generate the assessment outcome based on the received assessment of the social competency.

In some embodiments, the assessment module 128 may pass the assessment outcome to the records module 122 and the records module 122 may record the assessment outcome in the user record 130, at 222. In some embodiments, the data handling module 120 may provide the assessment outcome to the assessment platform shown on the output device 112 of the user interface 106.

In some embodiments, the assessment application 118 may further determine a diagnosis of social competency of the user based on the assessment outcome.

In some embodiments, the assessment application 118 may further determine a treatment plan based on the assessment outcome.

It will be appreciated that the steps of method 200 may be performed in any suitable order and are not limited to the order herein described. For example, step 224 may occur before, concurrently or after step 222.

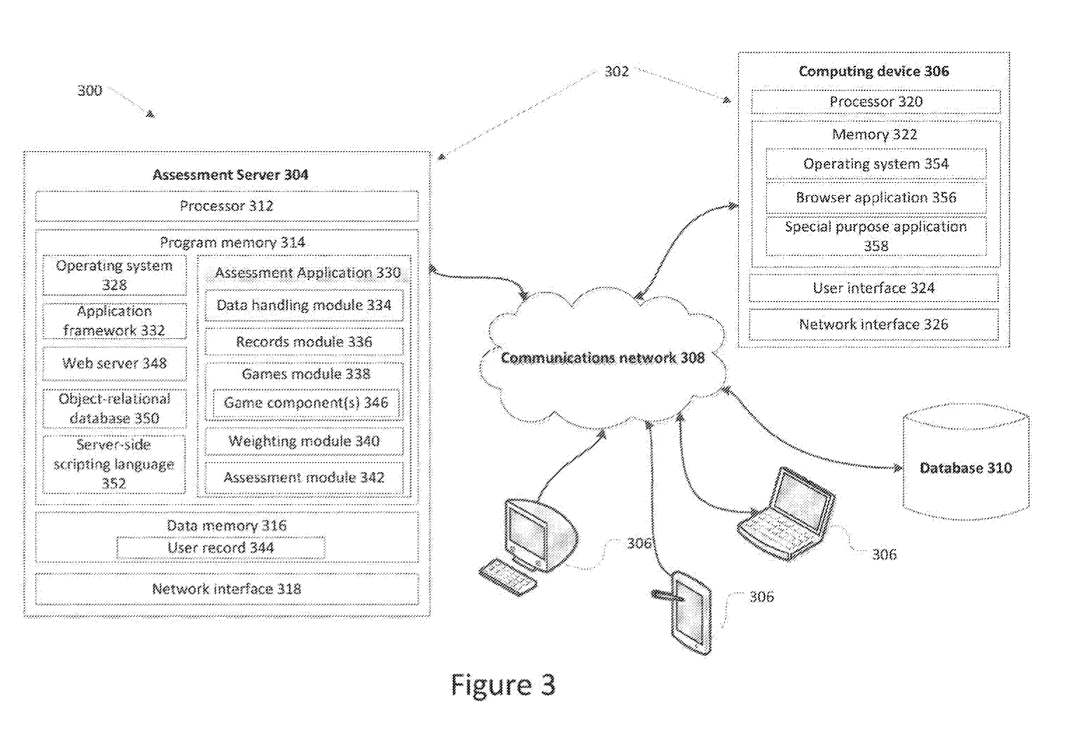

Referring now to FIG. 3, there is illustrated an example of a communications system 300 comprising an assessment system 302 comprising an assessment server 304 and a computing device 306 in communication with the assessment server 304 across a communications network 308, such as a local area network, a wireless data network, an intranet or the Internet or a combination of a number of such networks. The communications system 300 may further comprise a content file server or database 310 for storage and retrieval of data and/or one or more additional computing devices 306.

The database 310 may be coupled to and accessible by the assessment server 304 and/or the one or more computing devices 306, for example, directly or via a private communications network. In some embodiments, the database 310 may be coupled to and accessible by the assessment server 304 and/or the one or more computing devices 306 over the communications network 308. The database 310 may be configured to store data pertinent to operations of the assessment server 304 and provision of service to the computing devices 306. For example, the database 310 may store data pertinent to the assessment environment including game data, records of user details and personal information, statistical information, weightings etc.

In some embodiments, the assessment server 304 may comprise a server or server system, and may, in some embodiments, comprise multiple servers or server systems. In some embodiments, the computing device 306 may comprise a mobile or hand-held computing device such as a smartphone or tablet, a laptop, or PC, and may, in some embodiments, comprise multiple computer devices. In some embodiments, the communications system 300 comprises a client-server architecture where the assessment server 304 is configured as a server and at least one of the computing devices 306 is configured as a client device. For example, in some embodiments, in response to a request from one of the computing devices 306, the assessment server 304 is configured to execute an assessment application to provide an assessment environment or platform to the computing device 306 and to facilitate the assessment of the social competency of a user of the computing device 306 engaging with the assessment platform.

The assessment server 304 comprises a processor 312 (which may include multiple processing components or multiple virtual or physical servers operating together), program memory 314, data memory 316 and a network interface 318. The processor 312 may include components such as digital signal processing units (DSPUs), central processing units (CPUs), arithmetic logic units (ALUs) and registers for storing data. Program memory 314 and/or data memory 316 may comprise a combination of volatile and non-volatile computer readable storage. For example, program memory 314 may include read-only memory (ROM) such as erasable ROM (EROM) and electrically erasable programmable ROM (EEPROM or flash ROM), or random access memory (RAM) such as static RAM (SRAM), dynamic RAM (DRAM) or non-volatile RAM (NVRAM or flash). Program memory 314 has sufficient capacity to store program code executable by the processor 312 in order to perform appropriate processing functions as described herein, including providing an assessment environment or platform for facilitating the assessment of social competency of a user engaging in the assessment environment. Data memory 316 may be configured to store data pertinent to operations of the assessment server 304 and provision of service to the computing devices 306, and/or to store information pertaining to the assessment platform and the user of the assessment platform, such as the user records 130. The network interface 318 may comprise suitable hardware and software interfaces to facilitate wireless communication with computing devices 306 and servers 308, for example, over a network, such as communications network 310. For example, the network interface 318 may comprise a USB port, Ethernet port, a wireless adapter or a Bluetooth module. In some embodiments, the network interface 318 may comprise suitable hardware and software interfaces to facilitate wired communication between the assessment server 304, computing devices 306, and servers 308.

The computing device(s) 306 comprise at least one processor 320, one or more forms of memory 322, a user interface 324 and a network interface 326. Memory 322 may comprise volatile (e.g. RAM) and n-volatile (e.g. hard disk drive, solid state drive, flash memory and/or optical disc) storage. For example, memory 322 may store or be configured to store a number of software applications or applets executable by the at least one processor 320 to perform various device-related functions discussed herein. The user interface 324 may comprise at least one output device, such as a display and/or speaker, for providing an output for the computing device 306 and at least one input device, such as a touch-screen, a keyboard, mouse, microphone, video camera, stylus, push button, switch or other peripheral device that can be used for providing user input to the computing device 306. The network interface 326 may comprise suitable hardware and software interfaces to facilitate wireless communication with the assessment server 304, other computing devices 306 and/or servers 308, for example, over a network, such as communications network 310. For example, the network interface 326 may comprise a USB port, Ethernet port, a wireless adapter or a Bluetooth module. In some embodiments, the network interface 326 may comprise suitable hardware and software interfaces to facilitate wired communication between the computer device 306, the assessment server 304, other computing devices 104 and/or server(s) 308.

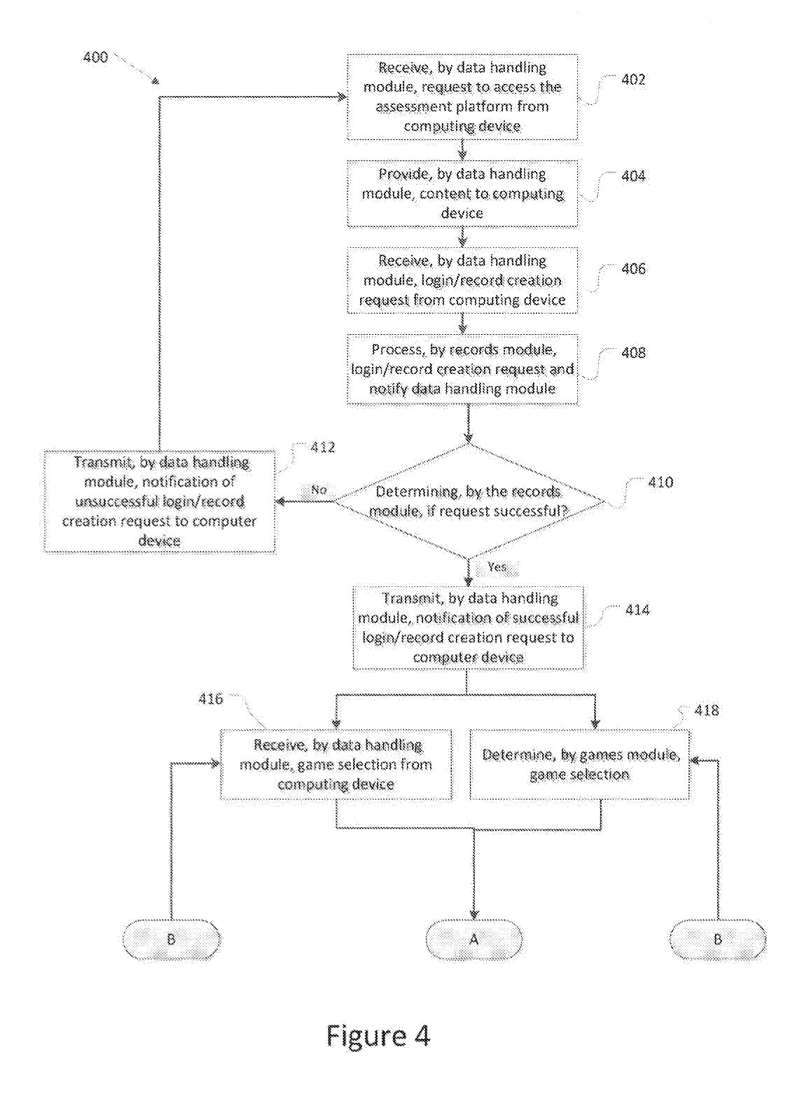

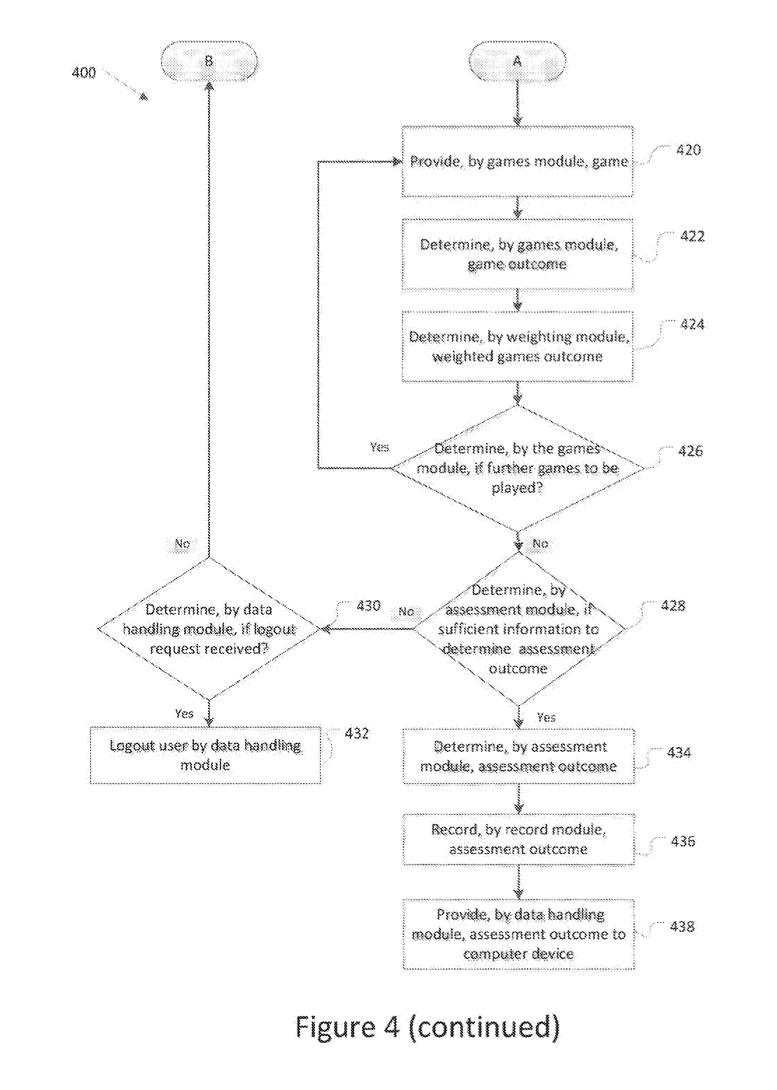

The program memory 314 of the assessment server 304 comprises program code including an operating system 328 and an assessment application 330 supported by suitable application framework 332, such as Symfony (for PHP). Similar to the assessment application 118 hosted by the assessment system 100 discussed above, the assessment application 330 comprises program code, such as executable code modules, which includes a data handling module 334, the records module 336, the games module 338, and the assessment module 342, and in some embodiments, the weightings module 340. The executable code modules of the assessment application 330, when aggregated and executed by the one or more processors 312, perform programmed functions as described herein and cause the assessment server 304 to provide the assessment platform to the computing device(s) 306 to facilitate the assessment of social competency of users of the assessment platform, as will be discussed in more detail with reference to FIGS. 4 to 44.

The data handling module 334, when executed by the processor(s) 312, may be configured to pass data such as requests and responses, between the computing device(s) 306 and the relevant module(s) of the assessment application 330.

The records module 336, when executed by the processor(s) 312, may be configured to create, store, modify and delete user records 344 associated with users of the assessment platform. Similar to user records 130, each user record 344 may comprise personal details of the user, such as name, age, contact information, information relating to social competency of the user, outcomes of games played on the assessment platform by the user, determinations made by the assessment module 342, etc. The user records 344 may be stored in data memory 316, in database 312 and/or locally at the computing device(s) 306, for example, in memory 322.

The games module 338, when executed by the processor(s) 312, may be configured to provide a plurality of interactive games to the user platform or environment. The games module 338 may comprise a plurality of interactive game components 346, each interactive game component, when executed by the processor(s) 312, may be configured to provide an interactive game to the user platform, to allow the user to play the game and to determine an outcome of the game played by the user. The outcome of the game may provide an effective indication or assessment of a specific skill or ability of the user. The interactive game components 346 may comprises similar components to interactive game components 132 of the assessment application 118 and are described in detail below with reference to FIGS. 7 to 42.

The weighting module 340, when executed by the processors 312, may be configured to applying weightings to outcomes of interactive games played by users of the assessment platform. For example, interactive games provided on the assessment platform may be accorded different levels of importance in assessing the social competency of a user engaging with the assessment platform or environment. Accordingly, the outcomes of the interactive games played by a user may be weighted before the assessment module 342 determines an assessment of the social competency of the user based on the outcomes of the interactive games played by the user.

The assessment module 342, when executed by the processor(s) 312, may be configured to determine an assessment of the social competency of a user engaging with the assessment platform. The assessment module 342 may base the assessment on the outcomes of at least some of the interactive games played by the user, and in some embodiments, weighted outcomes of the interactive games played by the user. In some embodiments, the assessment module 342 may base the assessment on the outcome of at least one interactive game associated with each of the social domains of attention/executive function, communication, and social cognition. The assessment module 342 may also employ data other than the outcomes of the interactive games in determining the assessment. For example, the assessment module 342 may employ data stored in the user record 344 associated with the user in determining the assessment. In some embodiments, the assessment module 342 may determine a social quotient or “social IQ score” for the user indicative of social competency of the user and which may be employed to guide professional interpretation of the user's social competency and identification of appropriate early intervention measures.

Program memory 314 may also comprise web server functions 348 (e.g. using a hypertext transfer protocol daemon (HTTP)), data processing functions and data storage and retrieval functions (e.g. using structured query language (SQL) support, such as object-relational database 350) in conjunction with database 312. Program memory 314 may also comprise scripting language support 352, such as Microsoft™ ASP, ASP.NET, Java/J2EE or PHP.

When supported by and combined with the application framework 332, and the other server-side functions described previously, the assessment application 330 provides an assessment platform or environment for assessing social competency of user engaging with the assessment platform. In some embodiments, the assessment platform may encompass activities that can be viewed as being performed or executed at the computing devices 306. However, it is to be emphasised that the activities performed or executed at the computing devices 306 are reliant on program code (including applets, where appropriate) served by assessment server 304 to the computing devices 306 and executed by a browser application 356. The functionality of the assessment platform is therefore driven by code executed and served by the assessment server 304.

Memory 322 of the computing device 306 includes an operating system 354 and a number of software applications or applets executable by the at least one processor 320 to perform various device-related functions. At least one such software application includes a browser application 356 for enabling a user to navigate to sites accessible over the communications network 308 to receive content therefrom. In some examples, the browser application 356 can be used to communicate with assessment server 304 to request content therefrom, in the form of one or more web pages provided as program code to the browser application 356 and executable by the processor 320. According to some embodiments, the assessment server 304 is configured to serve code to the computing device 306 to provide the assessment platform or environment generated by browser application 356 and viewable on a display of the user interface 324. Thus, the assessment server 304 can be considered to act as a social competency assessment system or to act as a host for such a system.

In some embodiments, the browser application 356 may be supplemented by a special-purpose add-on or may be substituted by special-purpose user software (i.e. an “app” for a smart-phone or tablet device) in order to perform the functions described herein or to facilitate such functions. For example, for a computing device 306, a special purpose application 358 may be downloaded from the assessment server 304 or a separate dedicated download server (not shown) and installed on the computing device 306. When such a special purpose application 358 is executing on the mobile user computing devices 306, it may facilitate appropriate formatting, interaction, display and input to enable the user interface functionality described herein. In this way, the special-purpose application 358 cooperates with the assessment server 304 to provide access to and interaction with the assessment platform or environment realised by the assessment application 330 implemented by the assessment server 304.

For simplicity of illustration, the assessment server 304 is generally described as serving code or one or more applets to the computing devices 306 to perform some of the described user interface functions. However, it should be understood that this includes the assessment server 304 transmitting code and/or data to (and receiving code and/or data from) the special-purpose application 358 executing on the computing device 306 to enable that special-purpose application 358 to provide the necessary or appropriate displays and interactive features (e.g. display of information fields, user selectable options, conference panels etc.) described herein. Other user software applications may execute on the computing device(s) 306 using the operating system 354.

Referring now to FIG. 4, there is shown a process flow diagram of a computer-implemented method 400 of assessing social competency according to some embodiments, the method 400 operable by the assessment server 304. The data handling module 334, the records module 336, the games module 338 and the assessment module 342, and in some embodiments, the weighting module 340, are executed by the processor(s) 312 in order to perform the functions of the method 400 as described.

In some embodiments, the method 400 begins with a login process. For example, in some embodiments, the data handling module 334 receives a request to access the assessment platform from the browser application 356, at 402. The data handling module 334 provides content to the browser application 356 to allow the browser application 356 to display the assessment platform on the user interface 324, at 404. In some embodiments, the assessment platform provides input text field(s) or selectable option(s) to allow a user to login to a previously created account with the assessment application 330 and/or to create an account with the assessment application 330. The assessment platform may also provide input text field(s) or selectable option(s) to allow information about the user to be recorded in the user's account of the user records 344, such as the user's name and age.

The data handling module 334 may receive a login and/or records creation request from the browser application 356, which may, for example, include user data input from the user details area of the assessment platform and passes the data to the records module 336 for processing, at 406. The records module 336 processes the user data to determine whether the login and/or records creation request was successful or unsuccessful, at 408. Depending on the type of data received, the records module 336 interacts with the user records to verify or reject the user login attempt, creates a new account for the user and/or add information to the user's account in the user records and sends a notification to the data handling module 334 and/or the games module 336 to signify that the login and/or records creation request was successful or unsuccessful.

If the data handling module 334 determines from the notification that the login and/or records creation request was unsuccessful at 410, the data handling module 206 may notify the browser application 356 accordingly, and may, for example, issue a prompt to the browser application 356 to cause the browser application 356 to display an unsuccessful login and/or records creations notification in the assessment environment on the user interface 324, at 412. If the data handling module 334 determines from the notification that a login attempt was successful or a user account was successfully created, at 410, the data handling module 334 may notify the browser application 356 accordingly, and may, for example, prompt to the browser application 356 to cause the browser application 356 to display a successful login and/or records creations notification in the assessment environment on the user interface 324, at 414.

However, it will be appreciated that in other embodiments, method 400 may be initiated without a login process.

In some embodiments, the assessment platform provides input text fields and/or selectable options to allow a user to select a game from a plurality of games displayed on the assessment platform. Thus, in some embodiments, the data handling module 334 receives a game selection from the browser application 356, at 416.

However, in some embodiments, once assessment application 330 has been initiated, for example, the login and/or records creation request is deemed successful by the records module 208, games may be automatically selected by the games module 210 of the assessment application 330 for the user, at 418. In some embodiments, the game(s) selected by the games module 338 for the user is based on information received from the records module 344 about the user.

In some embodiments, one or more games may be automatically selected by the assessment application 330 for the user and the user may be allowed to select other games from a plurality of user selectable games options provided on the assessment platform.

The games module 338 provides the interactive game to the assessment environment to allow the user to play the interactive game, at 420 and determines an outcome of the interactive game based on the user's inputs when engaging or playing the interactive game, at 422. The records module 336 may record the game outcome. In some embodiment, the games module 336 provides the interactive game to the assessment platform in response to the data handling module 334 receiving a game selection from the browser application 356 of the computing device 306 at 418 and passing it to the games module 338. In some embodiments, the games module 338 provides the interactive game to the assessment environment in response to the games module 338 automatically determining the interactive game for the user, at 420.

In some embodiments, the games module 336 passes the game outcome to the weighting module 340 and the weighting module 340 determines a weighted game outcome based on the game outcome and a game weighting, at 424. In some embodiments, different games may be accorded different weightings based on their deemed importance or priority in determining an assessment of the user's social competency. For example, the game weighting for a given game may depend on the other games which are been used to determine the assessment, whether the game is a basic or complex version, to which social domain type the game belongs, and/or the characteristics of the game. The game weighting may be stored in database 310 and/or data memory 316.

In some embodiments, where no weighting module 340 is employed, the games module 338 may pass the game outcome of the game directly to the assessment module 342.

The games module 338 determines whether further game(s) are to be played, at 436. If the games module 338 determines that a further game is to be played, for example, in response to receiving, by the data handling module 334, a further game selection from the browser application 356 and/or in response to determining, by the games module 338, an automatically selected further interactive game, the games module 338 provides the game selection of the interactive game to the assessment platform, at 420.

If the games module 338 determines that a further game is to be played, for example, in response to receiving, by the data handling module 334, a request for an assessment outcome, and/or in response to determining, by the games module 338, that no further interactive games are to be provided, the assessment module 342 determines whether there is sufficient information to determine an assessment outcome, at 428. For example, in some embodiments, the assessment module 342 determines whether a game outcome has been determined for at least one interactive game in each of the at least three social domains, i.e., attention/executive function, communication, and social cognition.

If the assessment module 342 determines that there is insufficient information to determine an assessment outcome, at 428, the data handling module 334 determines whether a logout or other session termination notification has been received from the browser application 356, at 430. If the data handling module 334 determines that a logout or other session termination notification has been received from the browser application 356, at 430, the data handling module 334 logs out the user and/or ends the session. In some embodiments, the records module 336 records the games outcomes determined during the session in the user record 344. If the data handling module 334 determines that a logout or other session termination notification has not been received from the browser application, at 430, the assessment application 330 determines a game selection at 416 and/or 418.

If the assessment module 342 determines that there is sufficient information to determine an assessment outcome, at 428, the assessment module 342 determines an assessment outcome based on the game outcomes and/or weighted game outcomes associated with the user, at 434. In some embodiments, the assessment module 342 may also base the assessment outcome on information in the user record, such as information derived from social skills questionnaires. In some embodiments, the assessment outcome comprises a social competency value such as a social quotient or social IQ.

The assessment module 342 may pass the assessment outcome to the records module 336 and the records module 336 may record the assessment outcome in the users record 344, at 436. In some embodiments, the data handling module 334 may provide the assessment outcome to the browser application 356 to cause the browser application 356 to display the assessment outcome on the assessment platform on the user interface 324. In some embodiments, the assessment module 342 may further determine a treatment plan based on the assessment outcome, which data handling module 334 may provide to the browser application 356 to cause the browser application 356 to display the treatment plan on the assessment platform on the user interface 324.

It will be appreciated that the steps of method 400 may be performed in any suitable order and are not limited to the order herein described. For example, step 424 may occur before, concurrently or after steps 426 and/or 428 Similarly, step 436 may occur before, concurrently or after step 438.

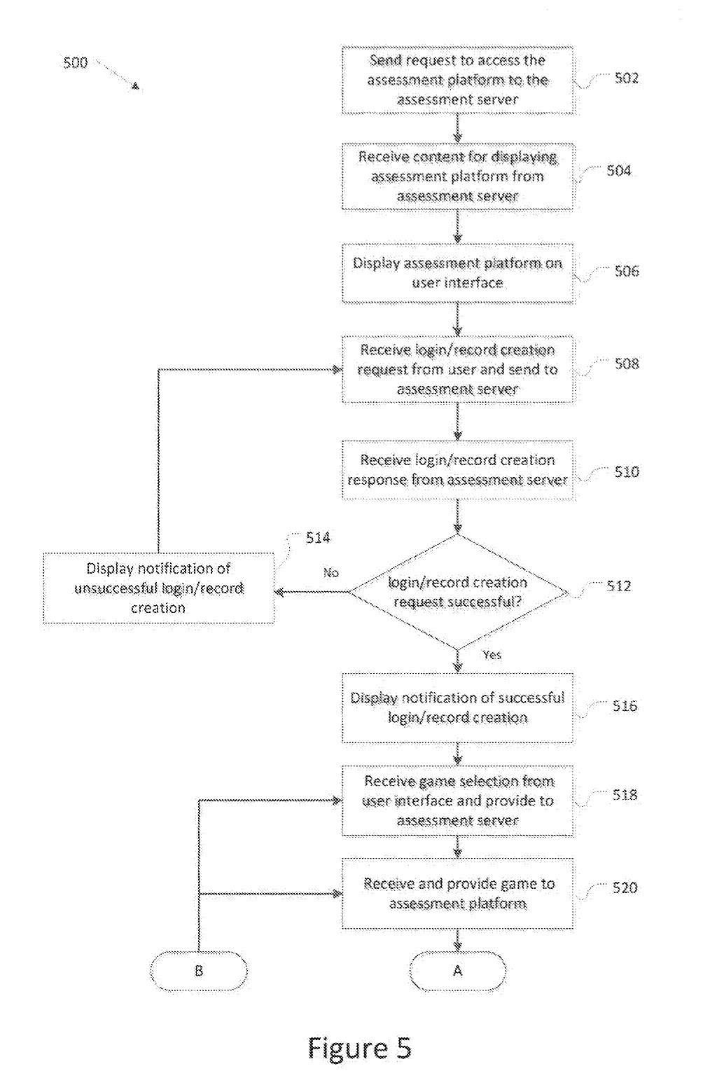

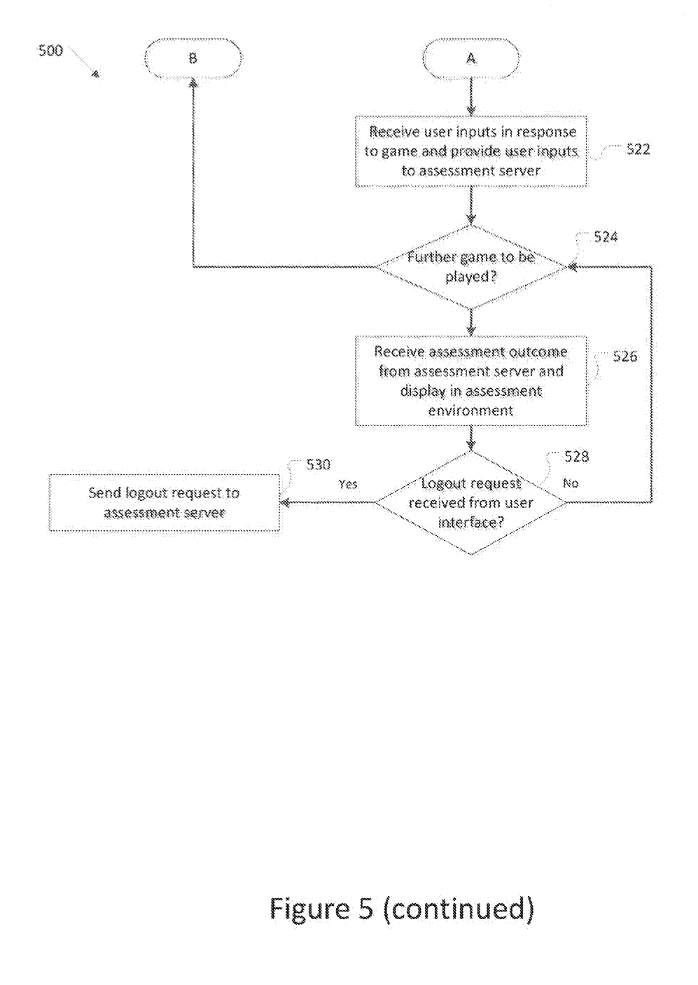

Referring now to FIG. 5, there is shown a process flow diagram of a computer-implemented method 500 of providing an assessment platform for assessing social competency according to some embodiments, the method 500 operable by one of the computing devices 306. The browser application 356 is executed by the processor 320 in order to perform the functions of the method 500 as described.

The method 500 begins with the browser application 356 sending a request to the assessment server 304 seeking access to the assessment platform, at 502. For example, the browser application 356 may send the request in response to receiving an input from the user interface 324. The browser application 356 receives content from the assessment server 304 for displaying the assessment platform on the user interface 324, at 504.

In some embodiments, the browser application 356 receives a login or record creation request from the user via the user interface 324 and transmits the request to the assessment server 304, at 508. The browser application 356 may then receive login or record creation response from the assessment server, at 510. If the browser application 356 determines that the login or record creation request was not successful, at 512, the browser application 356 may display a notification of an unsuccessful login or record creation request on the user interface 324 of the computing device 306, at 514. If the browser application 356 determines that the login or record creation request was successful, at 512, the browser application 356 may display a notification of a successful login or record creation request on the user interface 324 of the computing device 306, at 516.

In some embodiments, the browser application 356 receives an input indicative of a game selection of a game provided on the assessment platform from the user via the user interface 324 and provides the game selection to the assessment server 304, at 518. In response, the browser application 356 receives the selected game from the assessment server 304 and provides it to the assessment platform, at 520.

In some embodiments, as opposed to receiving the game selection form the user at 518, the assessment application 330 automatically determines a game selection for the user, as discussed above with reference to FIG. 5, and the browser application 356 receives the selected game from the assessment server 304 and provides it to the assessment platform, at 520.

The browser application 356 receives user inputs from the user via the user interface 324 as the user plays or engages with the game, and the browser application 356 transmits the user inputs to the assessment server 304, at 522.

The browser application 356 determines whether a further game is to be played, at 524. In some embodiments, the browser application 356 may query the assessment server 304 to determine whether a further game is to be played. In some embodiments, the browser application 356 and/or the assessment server 304 may determine whether a further game is to be played based on whether there is sufficient information to determine an assessment outcome. For example, in some embodiments, sufficient information to determine an assessment outcome requires a game outcome to have been determined for at least one interactive game in each of the at least three social domains, i.e., attention/executive function, communication, and social cognition. In some embodiments, the browser application 356 and/or the assessment server 304 may determine whether a further game is to be played based on whether the browser application 356 have received a user input indicative of a game selection.

If the browser application 356 determines that a further game is to be played, the method reverts to 520, and the browser application 356 receives the selected game from the assessment server 304 and provides it to the assessment platform, at 520. In some embodiments, the method may revert to 518 to allow the user to make a game selection.

If the browser application 356 determines that a further game is not to be played, the method reverts to 520, the browser application 356 receives an assessment outcome from the assessment server 304, at 526.

In some embodiments, the browser application 356 determines whether a logout request and/or other indication for termination of the assessment application is received from the user via the user interface 324, at 528. If the browser application 356 determines that no such logout or termination request has been received, the method 500 may revert to 524 to determine whether another game is to be played. If the browser application 356 determines that a logout or termination request has been received, the browser application 356 may send a request to the assessment server 304 to log out of the assessment platform, at 530.

It will be appreciated that the steps of method 500 may be performed in any suitable order and are not limited to the order herein described.

FIG. 6 is an example screen shot of a display 600 of the assessment platform displayed on the output device 112 of the assessment system 100 or the user interface 324 of the computing device 306 of the assessment system 302. The assessment platform may have a title 601, which may prompt the user to “Start a New Test”, for example. As shown, the assessment platform may comprise a plurality of input fields 602, 603 and 604. Input field 602 may be a participant identifier field, through which a user can input a unique identification number that is assigned to them, for example. Input field 603 may be a date of birth field, through which a user may be able to input their date of birth. Input field 604 may be a sex field, through which the user can input their sex or gender. The assessment platform may further comprise a number of selectable options 605, 606, 607, 608, 609 and 610 to allow a user to select an interactive game from a plurality of games and/or to provide data to the assessment application, such as user record data and/or data relevant to determining the normative data, as discussed above, using the user interface 106 or user interface 324.

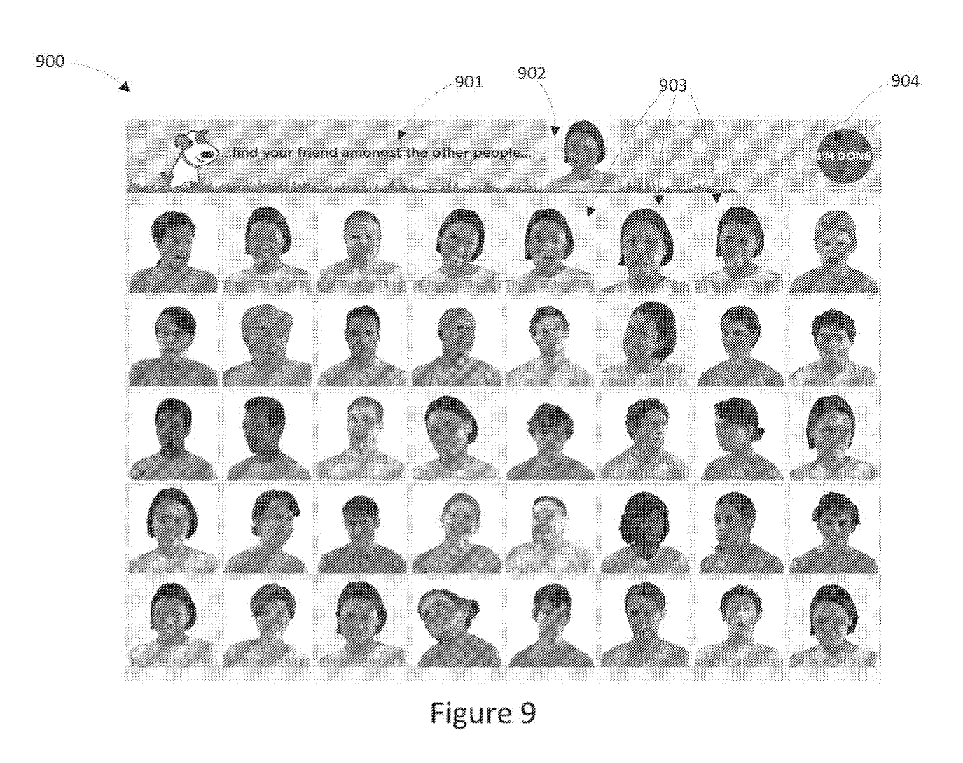

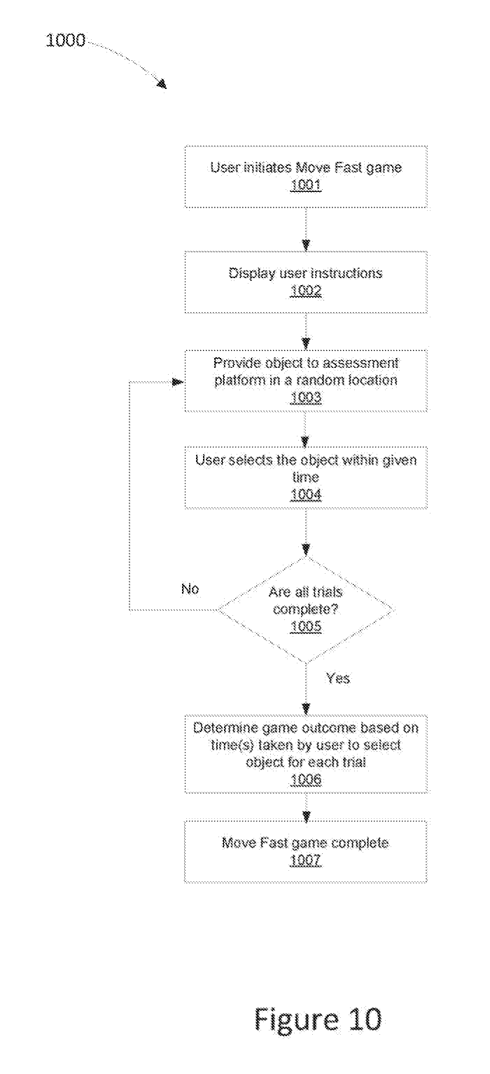

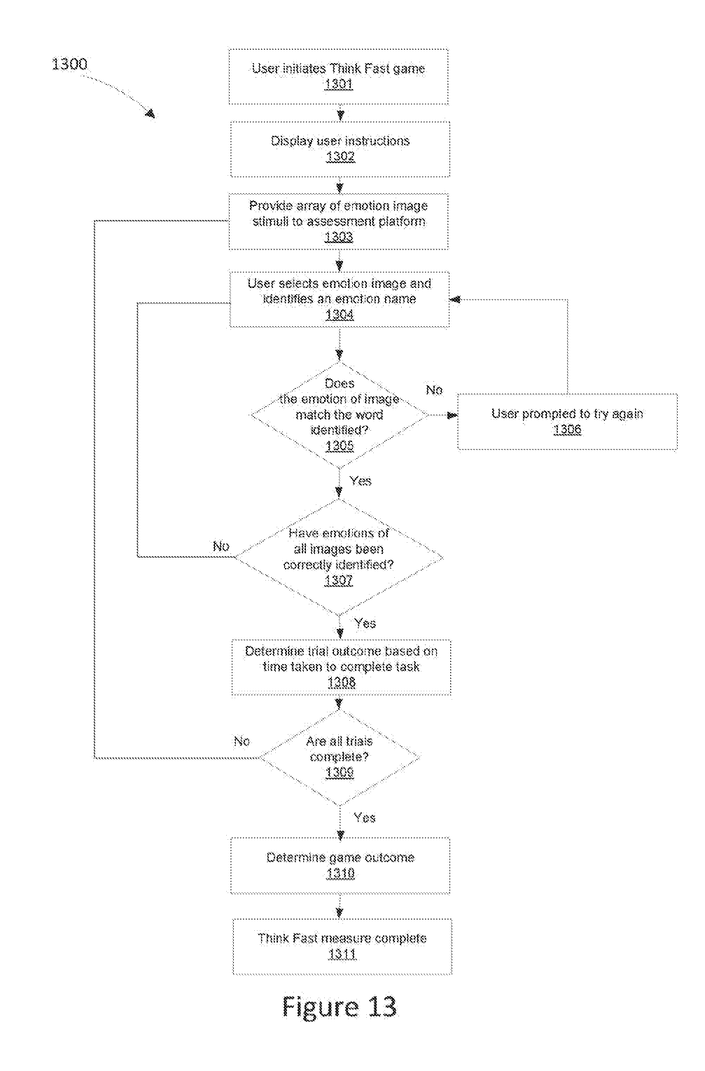

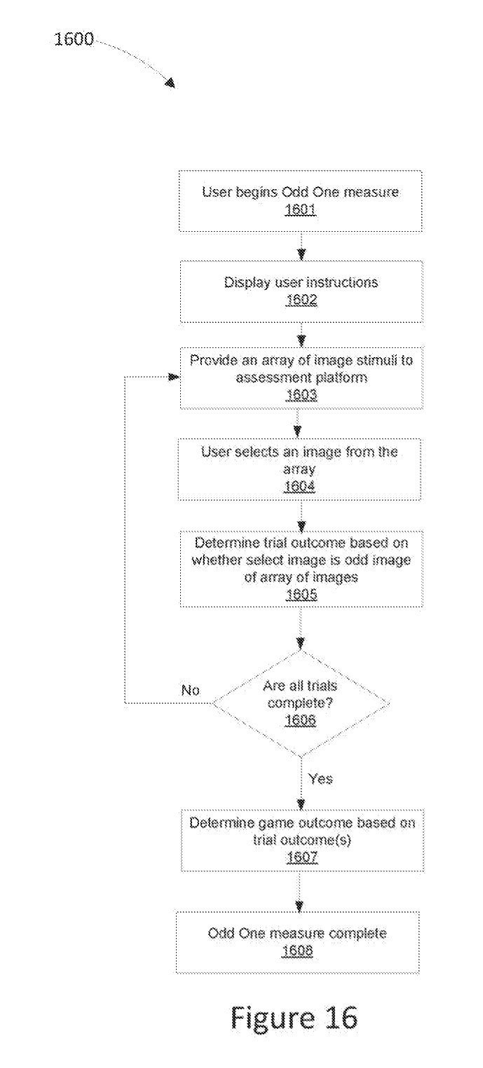

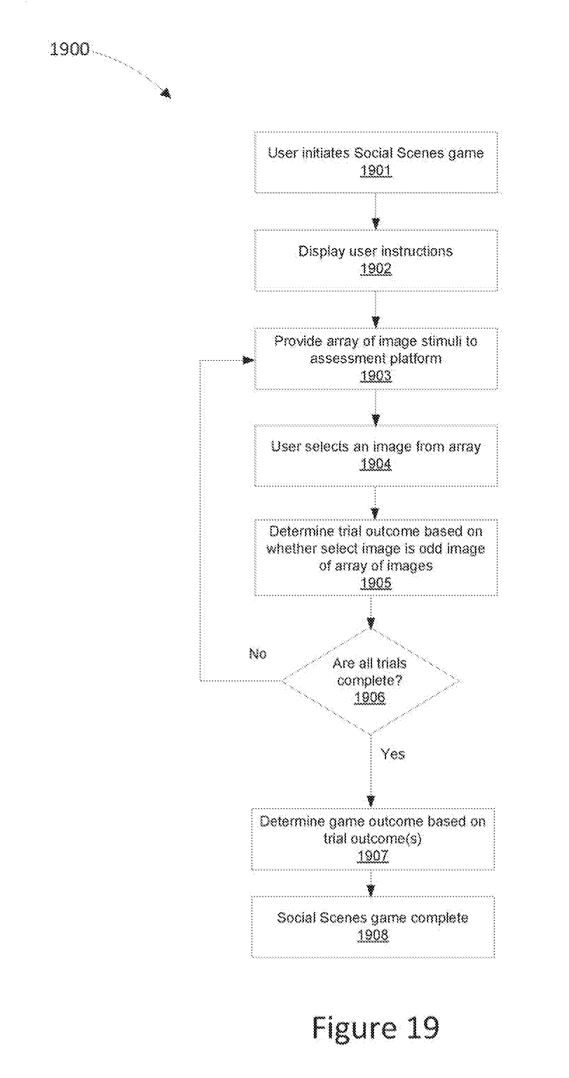

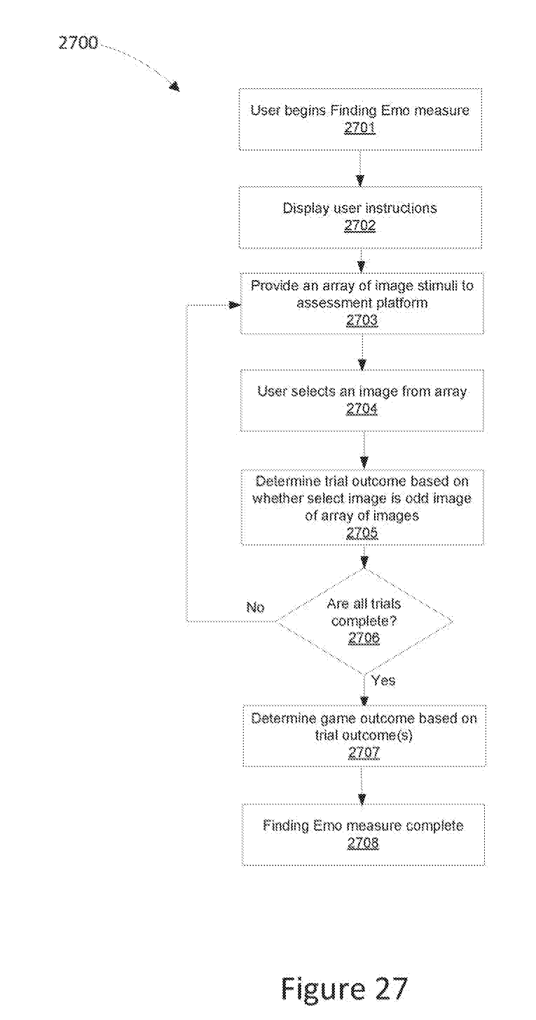

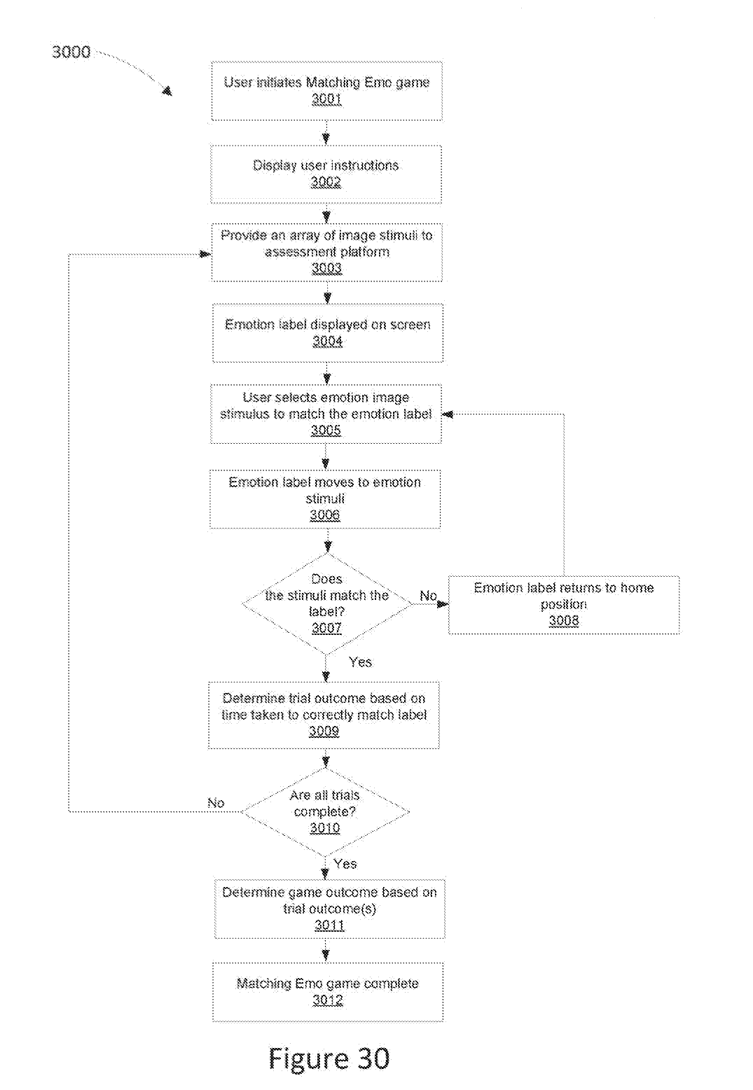

In the illustrated example, selectable option 605 may allow a user to navigate to a basic social cognition type game for determining a measure of a user's emotion perception of self and others, which may be called the “Matching Emo” game in some embodiments, as described below in further detail with reference to FIGS. 30 to 32. Selectable option 606 may allow a user to navigate to a basic social cognition type game for determining a measure of the user's ability to recognise emotion, which may be called the “Finding Emo” game in some embodiments, as described below in further detail with reference to FIGS. 27 to 29. Selectable option 607 may allow a user to navigate to a basic communication type game for determining a measure of the user's ability to comprehend non-verbal language, which may be called the “Odd One” game in some embodiments, as described below in further detail with reference to FIGS. 16 to 18. Selectable option 608 may allow a user to navigate to a basic communication type game for determining a measure of the user's ability to comprehend social intent, which may be called the “Social Scenes” game in some embodiments, as described below in further detail with reference to FIGS. 19 to 21. Selectable option 609 may allow a user to navigate to a basic attention/executive function type game for determining a measure of reaction time of the user, which may be called the “Move Fast” game in some embodiments, as described below in further detail with reference to FIGS. 10 to 12. Selectable option 610 may allow a user to navigate to a basic attention/executive function type game for determining a measure of speed and/or information processing of the user, which may be called the “Think Fast” game in some embodiments, as described below in further detail with reference to FIGS. 13 to 15.

Each selectable option 610 may include a logo 611, a title 612, and a description 613. Logo 611 may give a user of the assessment platform an idea as to the type of images used in each game. Title 612 may give an indication as to what the game may involve. Description 613 may go into further detail about the game. For selectable option 605, for example, title 611 shows one of the images used in the game. Title 612 reads “Matching Emo”, which gives the payer an indication that the game may involve matching emotions. Description 613 reads “This test will assess the emotional response of a child by matching random faces with descriptions”.

Social competence develops naturally through childhood and adolescence, starting with the infant's social smile and progressing to the adolescent's pre-occupation with peer interaction and acceptance. Social development may be underpinned by maturation of various skills, each contributing to ultimate social success. For example, skills which may be integral to social competence may include: i) attention and executive function: selective attention, rapid response, self-regulation, mental flexibility, adaptability; ii) social cognition: emotion perception and identification from facial expression and gestures, perspective taking, empathy, attribution of social intent, moral maturity; and iii) social communication: decoding body language, understanding pragmatics, irony and sarcasm. In some embodiments, the interactive games of the games module 124 of the assessment application 118 may be configured to determine or measure at least some of such social skills of the user.

Attention/executive function type games or tests are designed to determine a measure of attention/executive function such as the user's processing speed, attention, self-regulation and/or flexibility. For example, a basic attention/executive function type game may comprise a game configured to determine a measure of the user's selective attention, reaction time and/or speed and/or information processing. A more complex attention/executive function type game may comprise a game configured to determine a measure of the user's inhibition/self-regulation and/or social/mental flexibility, for example.

Selective attention allows an individual to attend to important tasks while ignoring irrelevant stimuli. Impairment in selective attention may result in difficulty concentrating on a task and engaging appropriately in a high stimulus social environment such as a school classroom. Research provides support for the negative social outcomes of selective attention impairment exemplified in attention deficit hyperactivity disorder.

Reaction time is a basic cognitive process, and underlies almost all higher order cognitive skills such as information processing speed, attention and self-regulation. The inclusion of reaction time in a cognitive battery allows for the ability to accurately account for motor skills deficits.

Speed of processing social information is important for keeping pace with conversation and social interactions. Slowed processing speed may result in negative social outcomes due to difficulty responding to fast-paced or complex social interactions.

An example of a basic attention/executive function type game for determining a measure of selective attention is represented by FIG. 7. FIG. 7 is a process flow diagram of a computer-implemented method 700 of determining a measure of selective attention of the user, according to some embodiments, the method 700 operable by the assessment system 100, 302, and in particular, execution of the processor(s) 102, 312 of an interactive game component 132, 346 of the games module 124, 338 of the assessment application 118, 330. In this example, the basic attention/executive function type game is referred to as FIND FRIEND.

Method 700 starts when a user begins the “Find Friend” game, at 701, for example, by selecting selectable option 605 on the assessment platform. In some embodiments, processor 102, 312 executes an interactive game component 132, 346 that causes a set of instructions to be shown to the user, at 702, to direct the user to identify a target image or photo from a selection or array of image or photo stimuli. For example, the set of instructions may include an instruction such as “find your friend amongst the other people”, including a picture of the “friend” the user is to find. Processor 102, 312 causes an array of photo stimuli to be shown on the screen, which includes at least one picture of the target image or “friend” to be identified by the user, at 703. The user selects an image stimulus from the array that they believe to match the target image, at 704. In some embodiments, processor 102, 312 monitors user inputs to determine whether the user has selected an “I'm Done” button to show that they have made their selection, at 705. If not, the method goes back to step 704. In other embodiment, the selection of an image stimulus is sufficient to indicate to the games module 124, 338 that the user has made their selection. If the user does select the “I'm Done” button, or the processor otherwise determines that the user has made their selection, processor 102, 312 determines or checks to see whether all trials are complete at step 706. For example, in some embodiments, the “Find Friend” game may comprise a plurality of rounds or trials. If more trials are to be performed, a new set of image stimuli are generated and provided to the assessment platform, at 703. Once all trials are complete, the games module 124, 338 determines the game outcome based on the selected image(s) input by the user. For example, for each trial, the games module 124, 338 compares the selected image with the target image to determine whether the user has correctly identified the target image amongst the array of images displayed. In some embodiments, the game outcome comprises a score indicative of a percentage of correctly identified images. In some embodiments, the games module 124, 338 may be further configured to monitor a length of time taken by the user to make the selection and the game outcome may be further based on time taken to make the selection. The method then moves to 708, where the Find Friend game is considered to be complete. Processor 102, 312 may cause the Find Friend game to exit at step 708.

According to some embodiments, the Friend Find game assesses selective attention through two or more trials. For example, some embodiments may have two, three, four, five, six, or more trials. During each trial, a number of images are shown arranged on the screen, and a target image is displayed in the middle top screen. Some embodiments may have 40 images displayed in an 8 by 5 grid. Some alternative embodiments may have 20, 25, 30, 35, or another number of images, which may be arranged on the screen in a grid or in another arrangement on the screen.

Each image may display a person facing the user. The person may be facing the user straight on, or angled slightly to the right or left. The image may show the person from the shoulders up, and the person may be placed on a white background in some embodiments. In each trial, a number of the images may display the same person as the target image. For example, in some embodiments, 12 of the displayed images may show the same person as the target photo. Each photo may display a different emotion. The user may be asked to select as many images as they can find showing the same person as the target image. They are asked to do this as quickly as possible, then select “I'm done” when they have finished. In some embodiments, the Find Friend game may take approximately two minutes to complete. In some alternative embodiments, the Find Friend game may take approximately four, six, eight, ten, or more minutes to complete.

In some embodiments, the Find Friend game may include a practice round, to allow the user to become familiar with the format of the game. During the practice round, an assessor may explain the Find Friend game to the user, explaining that the target image is an image of their “friend”, and that they must tap all of the images of their friend that appear on the screen as quickly as they can. The assessor may play one round of the game as a demonstration to the user. The assessor may then ask the user to try it. In some embodiments, the practice round may show 12 images arranged on the screen, plus the target image. In some embodiments, the practice round may show 10, 14, 16, 18, 20, or another number of images on the screen. The number of images showing the same person as the person in the target image may be one, two, three, four, five or more in some embodiments. The practice round may include a number of trials, such as one, two, three, four, five or more trials. Once the practice round is complete, the assessed phase of the Find Friend game may begin, as described above with reference to FIG. 7.

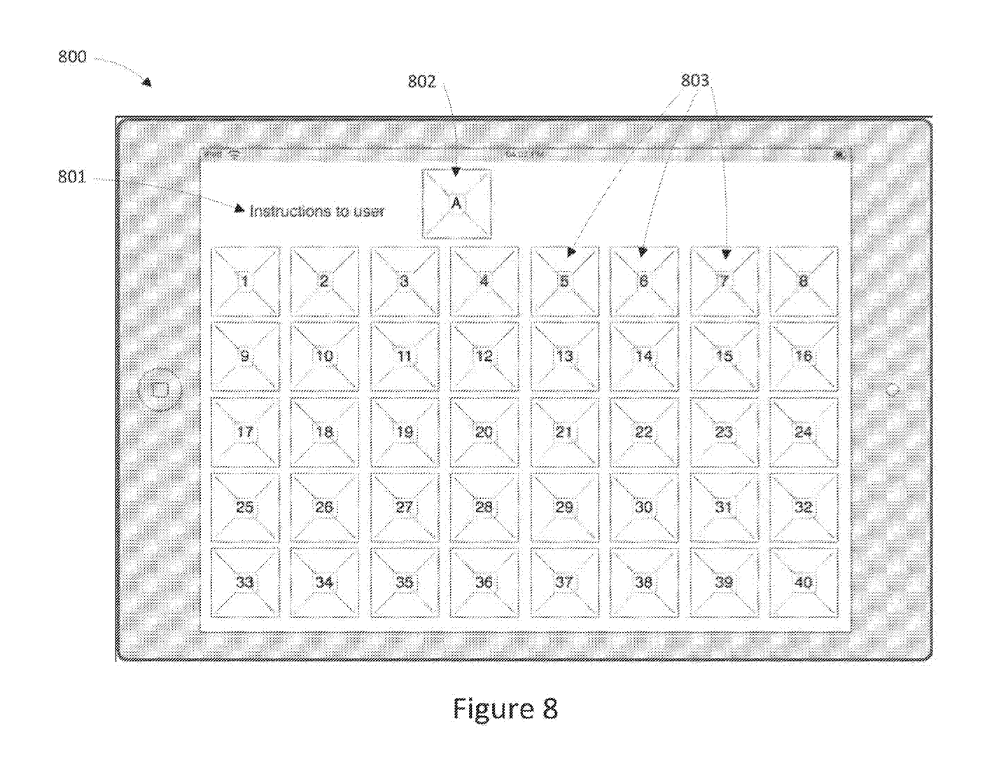

An example screen layout 800 for the “Find Friend” game is shown in FIG. 8. Screen layout 800 includes instructions 801, a primary picture 802, and an array of pictures 803. Instructions 801 may prompt a user to select a picture from array 803 that matches primary picture 802. Primary picture 802 and the pictures in array 803 may be photos of human faces in some embodiments.

An example screenshot 900 for the “Find Friend” game is shown in FIG. 9. Screenshot 900 includes instructions 901, a primary picture 902, an array of pictures 903, and an “I'm done” button 904. In the illustrated embodiments, instructions 901 prompt a user to “find your friend amongst the other people”. In the illustrated embodiment, primary picture 902 and the pictures in array 903 are pictures of human faces showing a variety of emotions. Once a user has made their selection, they may be able to indicate this by pressing the “I'm done” button 904.

An example of a basic attention/executive function type game for determining a measure of reaction time of the user is represented by FIG. 10. FIG. 10 is a process flow diagram of a computer-implemented method 1000 of determining a measure of reaction time, according to some embodiments, the method 1000 operable by the assessment system 100, 302, and in particular, execution of the processor(s) 102, 312 of an interactive game component 132, 346 of the games module 124, 338 of the assessment application 118, 330. In this example, the basic attention/executive function type game is referred to as MOVE FAST.

Method 1000 starts when a user initiates the “Move Fast” game, at 1001, for example, by selecting selectable option 605 on the assessment platform. In some embodiments, processor 102, 312 executes an interactive game component 132, 346 that causes a set of instructions to be shown to the user, at 1002, which may include an instruction such as “tap the object as fast as you can”. Processor 102, 312 causes an object to be displayed on the assessment platform shown on a screen of the user interface 106, 324, at 1003. The object is positioned at one of a plurality of locations on the assessment platform in an apparently random location. The user selects a location on the assessment platform with a view to selecting the location of the object on the screen, and processor 102, 312 records the time taken for them to do this, at 1004. Processor 102, 312 checks to see whether all rounds or trials are, at 1005. For example, in some embodiments, the “Move Fast” game may comprise a plurality of rounds or trials. If more trials are to be performed, an object is displayed at a new location on the assessment platform, at 1003. Once all trials are complete, the games module 124, 338 determines the game outcome based on the time taken by the user to correctly identify the location of the object for each round, at 1006. For example, in some embodiments, the games module 124, 338 may be configured to record the time taken by the user to select a location and to compare the selected location with the position of the object. If the selected location of the object corresponds substantially with the position of the object, the round outcome for the game comprises the recorded time. If the selected location of the object does not correspond substantially with the position of the object, the round outcome for the game comprises a timed out or error value. The method then moves to step 1007, where the Move Fast measure is considered to be complete. Processor 102, 312 may cause the Move Fast game to exit at step 1007.

According to some embodiments, the Move Fast game assesses reaction time across 1 or more trials, which may be 2, 3, 4, 5, 6, or more trials in some embodiments. In each trial, an array of around 40 items may be shown on the screen, which may be around 20, 25, 30, 35, 45 or 50 items in some embodiments. Each item may consist of an illustrated object appearing in a random location on the screen. The object may be one of a number of stored illustrated objects. In some embodiments, there may be 2, 3, 4, 5, 6, 7, 8, 9, 10, or more stored illustrated objects that may be randomly selected for display. Reaction time may be measured by how quickly the user taps the illustrated object once it appears on the screen. According to some embodiments, the Move Fast game may take approximately two minutes to complete. In some alternative embodiments, the Move Fast game may take approximately four, six, eight, ten, or more minutes to complete.

The game outcome or measure of reaction time of the user may be determined based on a number of measured results of the Move Fast game. For example, the total number of items completed, the percentage of the available items completed, the response time for each individual item, and the total time taken to complete the game may be measured and used to determine a measure of reaction time of a user.

In some embodiments, the Move Fast game may include a practice round, to allow the user to become familiar with the format of the game. During the practice round, an assessor may explain the Move Fast game to the user, telling the user to tap the moving object as fast as they can. The assessor may play one round of the game as a demonstration to the user. The assessor may then ask the user to try it. In some embodiments, the practice round may generate 8 objects for the user to tap. In some embodiments, the practice round may generate around 2, 4, 6, 10, or more objects for the user to tap. In some embodiments, the practice round may consist of one trial. In some other embodiments, the practice round may have 2, 3, 4, 5, or more trials. Once the practice round is complete, the assessed phase of the Move Fast game may begin, as described above with reference to FIG. 10.

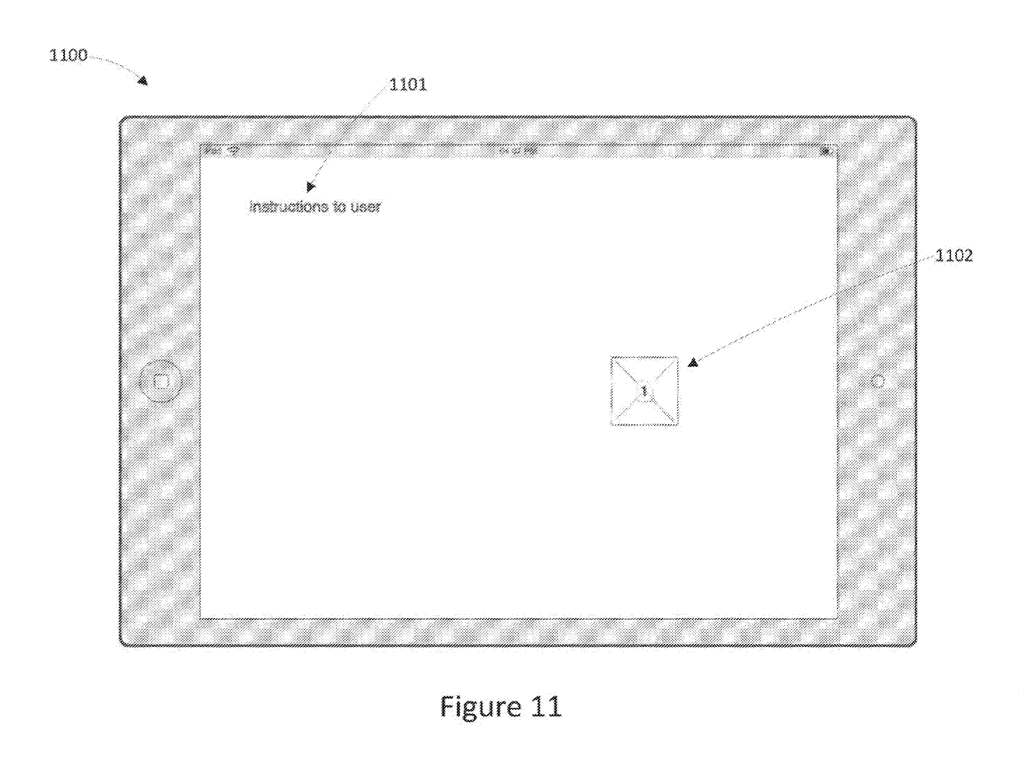

An example screen layout 1100 for the “Move Fast” game is shown in FIG. 11. Screen layout 1100 includes instructions 1101 and an object 1102. Instructions 1101 may prompt a user to select the object 1102 as quickly as they can. Object 1102 may be a computer generated image, symbol or graphic in some embodiments, such as an image of a ball, a square shape, or another visual object.

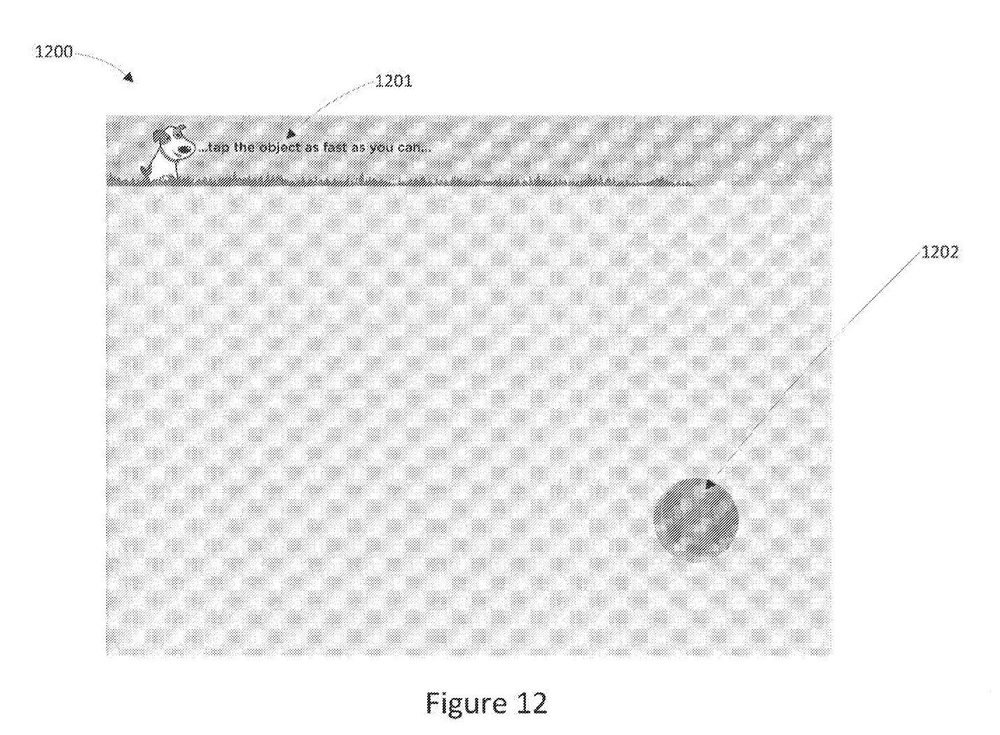

An example screenshot 1200 for the “Move Fast” game is shown in FIG. 12. Screenshot 1200 includes instructions 1201 and an object 1202. In the illustrated embodiments, instructions 1201 prompt a user to “tap the object as fast as you can”. In the illustrated embodiment, object 1202 is a computer generated image of a ball.

An example of a basic attention/executive function type game for determining a measure of speed and/or information processing of the user is represented by FIG. 13. FIG. 13 is a process flow diagram of a computer-implemented method 1300 of determining a measure of speed and/or information processing, according to some embodiments, the method 1300 operable by the assessment system 100, 302, and in particular, execution of the processor(s) 102, 312 of an interactive game component 132, 346 of the games module 124, 338 of the assessment application 118, 330. In this example, the basic attention/executive function type game is referred to as THINK FAST.

Method 1300 starts when a user initiates the “Think Fast” game, at 1301, for example, by selecting selectable option 605 on the assessment platform. In some embodiments, processor 102, 312 executes an interactive game component 132, 346 that causes a set of instructions to be shown to the user on the assessment platform, at 1302, which may include an instruction such as “tap and name the emotion”. Processor 102, 312 causes an array of emotion image stimuli to be provided to the assessment platform shown on a screen of the user interface 106, 324, at 1303. For example, the emotion image stimuli may include cartoon images of faces, photos or illustrations showing a variety of emotions. The user selects an emotion image stimulus, and names or identifies the emotion that they believe the image shows, at 1304. In some embodiments, an assessor assisting or monitoring the user participating in the game checks whether the emotion identified or described by the user matches the selected emotion image stimulus, at 1305. In some embodiments, processor 102, 312 executing the interactive game component 132, 346 determines whether the user identified the correct emotion. For example, the user may provide an input indicative of the identified emotion to the assessment platform via the user interface 106, 324, which may be processed by the processor 102, 312 to determine whether the identified emotion matches the selected emotion depicted by the emotion image stimulus. The user may enter the input by entering it into a text box provided on the assessment platform, making a selection from a list of possible emotions provided on the assessment platform, and/or vocally providing an audio input to the assessment platform and processor 102, 312 may determine whether the correct emotion was named based by processing the input from the user. If the user's identified emotion does not correspond substantially with the emotion depicted by the selected emotion image stimulus, the assessment platform prompts the user to reattempt to identify the emotion associated with the selected emotion image stimulus, at 1306 and the method returns to 1304. In some embodiments, the incorrectly identified emotion and selected emotion image stimulus are recorded. If the image stimulus does match or substantially correspond with the emotion identified by the user, processor 102, 312 executes the interactive game component 132, 346 to determine whether emotions associated with all of the emotion image stimuli of the array have been correctly identified by the user, at 1307 and if not, the user is prompted to select the text emotion image in the array, at 1304. If all of the emotion image stimuli of the array have been correctly identified, at 1307, the interactive game component 132, 346 determines the outcome of the trial. For example, the outcome of the trial may be based on one of more of the following factors: the percentage of correctly identified emotions on a first attempt; percentage of correctly identified emotions on a second, third and/or fourth attempt; the overall time taken to complete the trial; and whether or not the trial was successfully completed. Processor 102, 312 then executes the interactive game component 132, 346 to determine if more trials are to be performed, at 1309. If one or more trials are to be performed, a new set of emotion image stimuli are generated and provided to the assessment platform, at step 1303. If no more trials are to be performed, processor 102, 312 then executes the interactive game component 132, 346 to determine the game outcome based on the trial outcome of the one or more trials, at 1310. Once all trials are complete, the method moves to step 1311, where the Think Fast measure is considered to be complete. Processor 102, 312 may cause the Think Fast game to exit at step 1311.

According to some embodiments, the Think Fast game assesses information processing speed across 1 or more trials, which may be 2, 3, 4, 5, 6, or more trials in some embodiments. Each trial may consist of showing a number of illustrated faces on the screen. There may be 20, 25, 30, 35, 40, 45 or 50 faces shown on the screen in some embodiments. The faces may be shown in a grid, might may be a eight by five grid in some embodiments, or the faces may appear on the screen in a different arrangement. Each illustrated face may show one of a selection of emotions. In some embodiments, each face may show one of three emotions, which may be happy, sad, or angry. In some embodiments, each face may show one of four, five, six, or more emotions.

Each emotion may be displayed in random order a number of times on the screen. For example, each emotion may appear 5, 6, 7, 8, 9, 10, 11, 12, 13, 14 or 15 times on the screen in some embodiments. The user may be asked to tap each illustrated face and the name the emotion shown, starting from left to right and top to bottom. In some embodiments, the user may be asked to start at a different location on the screen, or to select the faces at random. Once tapped, the face may fade to grey to signal that it has been completed. If the user incorrectly names an emotion, the assessor may tap the incorrect face and asks the user to start again from that point. The Think Fast game may take approximately two minutes to complete. In some alternative embodiments, the Think Fast game may take approximately four, six, eight, ten, or more minutes to complete.

The game outcome or measure of information processing speed of the user may be determined based on a number of measured results of the Think Fast game. For example, the total number of items completed, the percentage of the available items completed, the number of correct answers, the number of attempts at a correct answer, the response time for each individual item, and the total time taken to complete the game may be measured and used to determine a measure of information processing speed of a user.

In some embodiments, the Think Fast game may include a practice round, to allow the user to become familiar with the format of the game. During the practice round, an assessor may explain the Think Fast game to the user, telling the user to quickly name and tap the faces. The assessor may tell the user the names of the emotions that each face may display. The assessor may direct the user to start on a new row once they have completed one row of faces. The assessor may also tell the user that if they incorrectly name the emotion, they will need to go back and try to name the emotion again. In some embodiments, the assessor may play one round of the game as a demonstration to the user. The assessor may then ask the user to try it. In some embodiments, the practice round may generate 8 faces for the user to name. In some embodiments, the practice round may generate around 2, 4, 6, 10, or more faces for the user to name. In some embodiments, the practice round may consist of one trial. In some other embodiments, the practice round may have 2, 3, 4, 5, or more trials. Once the practice round is complete, the assessed phase of the Think Fast game may begin, as described above with reference to FIG. 13.

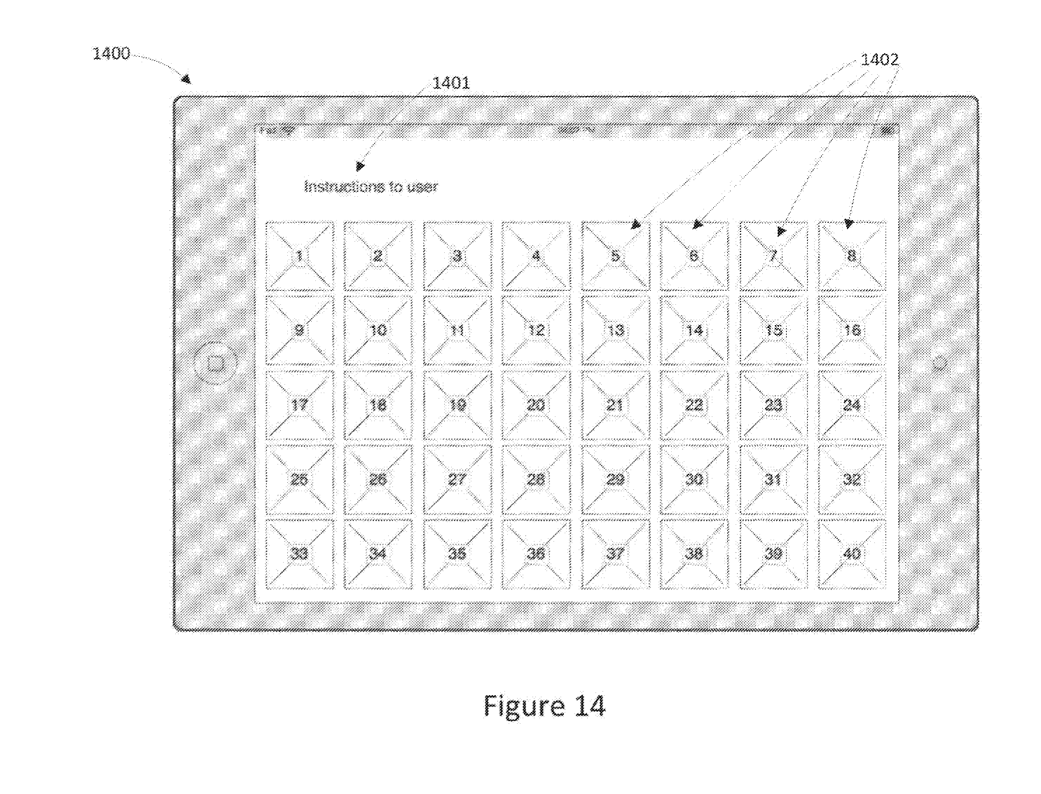

An example screen layout 1400 for the “Think Fast” game is shown in FIG. 14. Screen layout 1400 includes instructions 1401, and an array of emotion images 1402. Instructions 1401 may prompt a user to select an image from array 1402 and name the emotion that the image depicts. Images in array 1402 may be cartoon images of human faces in some embodiments.

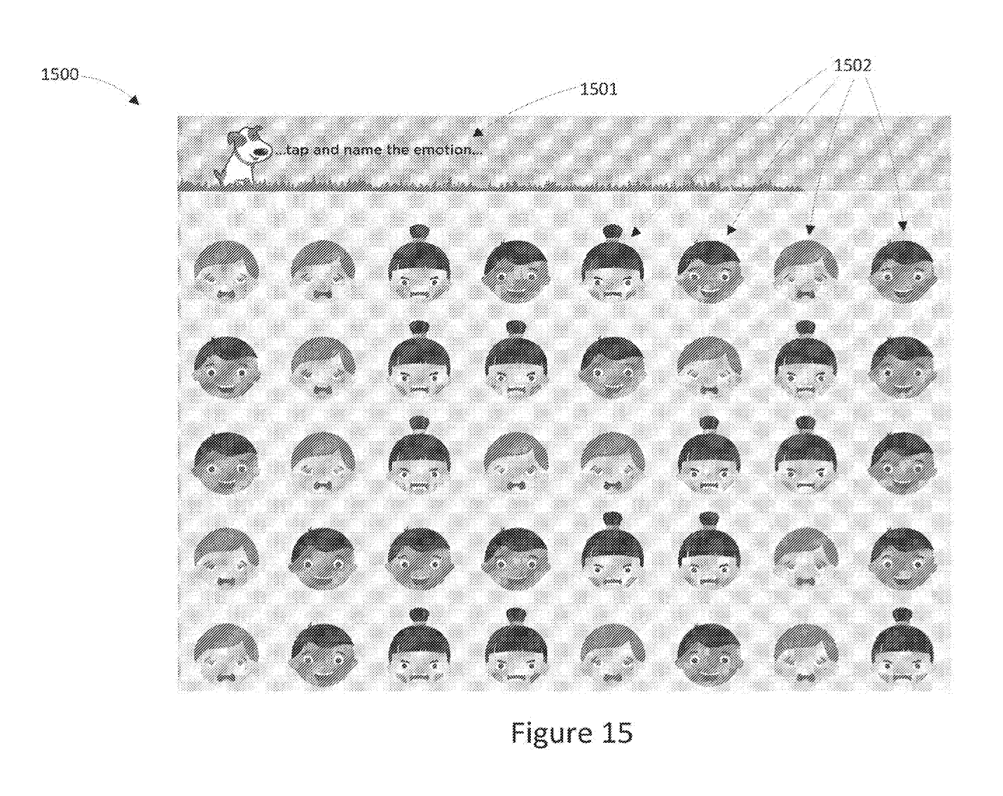

An example screenshot 1500 for the “Think Fast” game is shown in FIG. 15. Screenshot 1500 includes instructions 1501, and an array of pictures 1502. In the illustrated embodiments, instructions 1501 prompt a user to “tap and name the emotion”. In the illustrated embodiment, images in array 1302 are cartoon images of human faces showing a variety of emotions.

Communication type games or tests are designed to determine a measure of the user's comprehension of communication such as pragmatic language, prosody, and/or non-verbal language. For example, a basic communication type game may comprise a game configured to determine a measure of the user's ability to comprehend non-verbal language, for example, by decoding/processing body language and signs and/or non-verbal gestures, and/or social intent. A more complex communication type game may comprise a game configured to determine a measure of pragmatics, such as language use in social context and/or social intention, and/or prosody, such as use and understanding of irony and/or empathy.

As with perception and recognition of facial expression, processing nonverbal gestures involve interpreting body language to respond appropriately. Impairment in processing non-verbal gestures similarly affect ability to engage in appropriate reciprocal interaction and may result in negative social outcomes

The processing of social intention in an important step in the social communication process; appropriate behavioural responses are related to appropriate interpretation of social intention. Attribution of intention to ambiguous social interactions is influenced by an individual's temperament, interpersonal relationships, education and social learning.

Social interpretation is important for understanding language within a social context, such as turn-taking, regulation of appropriateness, and understanding context.

Prosody recognition refers to interpretation of non-linguistic signals such as intonation, pitch and rhythm and is necessary for understanding nuances of speech such as irony, emphasis and clarification, and the emotional state of the speaker. Impairment in prosody may result in confusion and responses inappropriate to the social situation.

An example of a basic communication type game for determining a measure of the user's ability to comprehend non-verbal language, for example decoding/processing body language and signs and/or non-verbal gestures, is represented by FIG. 16. FIG. 16 is a process flow diagram of a computer-implemented method 1600 of determining a measure of non-verbal language comprehension, according to some embodiments, the method 1600 operable by the assessment system 100, 302, and in particular, execution of the processor(s) 102, 312 of an interactive game component 132, 346 of the games module 124, 338 of the assessment application 118, 330. In this example, the basic communication type game is referred to as ODD ONE.

Method 1600 starts when a user initiates the “Odd One” game, at 1601, for example, by selecting selectable option 605 on the assessment platform. In some embodiments, processor 102, 312 executes an interactive game component 132, 346 that causes a set of instructions to be shown to the user, at 1602, which may include an instruction such as “tap the odd one out”. Processor 102, 312 causes an array of image stimuli to be displayed on the assessment platform shown on a screen of the user interface 106, 324, at 1603. For example, the array of image stimuli may include a number of images or photos of human faces showing various emotions or headshots with faces showing various emotions. In some embodiments, one of the image stimulus (the odd one) will be showing one emotion, and the remaining image stimulus will be showing a second, different emotion. The user selects an image stimulus from the array that they believe to be the odd one out from the group, at 1604. Processor 102, 312 determines the round or trial outcome based on a comparison of the selected image stimulus with the odd image stimulus, i.e., the image stimulus depicting an emotion different from that of the other image stimuli of the array, 1605. For example, if the selected image stimulus corresponds with the odd image stimulus, the trial outcome comprises an indication that the input provided was correct and if the selected image stimulus does not correspond with the odd image stimulus, the trial outcome comprises an indication that the input provided was incorrect. In some embodiments, the trial outcome may further comprise the selected image stimulus and the odd image stimulus, which may be employed by the assessment module 128, 342 or other component of the assessment application 118, 330 or assessment system in diagnosing the social competency of the user. For example, such information may provide an indication that a user has difficulties in distinguishing between two or more specific emotions in particular. Processor 102, 312 checks to see whether all trials are complete, at 1606. If more trials are to be performed, a new array of image stimuli are generated and provided to the assessment platform, at 1603. Once all trials are complete, the games module 124, 338 determines the game outcome based on the trial outcome(s), at 1607. The method moves to 1608, where the Odd One measure is considered to be complete. Processor 102, 312 may cause the Odd One game to exit at 1608.

According to some embodiments, the Odd One game assesses a user's ability to comprehend non-verbal language across 1 or more trials, which may be 2, 3, 4, 5, 6, or more trials in some embodiments. In some embodiments, the Odd One game may have 24 trials. Each trial may cause a number of preselected photos to be displayed. In some embodiments, 2, 3, 4, 5, 6, 7, 8 or more images may be displayed. The images may be displayed in a grid, which may be a two by two grid in some embodiments, or they may be arranged in a different configuration on the screen. All but one of the images may show people displaying one emotion, while one image may show a person displaying a second emotion. Each image may display a full-body image of a person facing the user. They may be facing the user straight on, or at a left or right angle. The person in the image may appear on a white background. The user may be asked to select the image displaying the odd emotion in each trial. The Odd One game may have a number of easy trials and a number of hard trials. In some embodiments, the game may have an equal number of easy and hard trials. The Odd One game may take approximately five minutes to complete. In some embodiments, the game may take approximately two, three, four, six, seven, eight, nine, ten, or more minutes to complete.

The measure of a user's ability to comprehend non-verbal language may be determined based on a number of measured results of the Odd One game. For example, the total number of items completed, the percentage of the available items completed, the number of correct answers, the response time for each individual item, the total time taken to complete the game, and the user's verbal explanation may be measured and used to determine a measure of information processing speed of a user.

In some embodiments, the Odd One game may include a practice round, to allow the user to become familiar with the format of the game. During the practice round, an assessor may explain the Odd One game to the user, telling the user to tap the odd one out. In some embodiments, the assessor may play one round of the game as a demonstration to the user. The assessor may then ask the user to try it. In some embodiments, the practice round may consist of three trials. In some other embodiments, the practice round may have 1, 2, 4, 5, or more trials. Once the practice round is complete, the assessed phase of the Odd One game may begin, as described above with reference to FIG. 16.

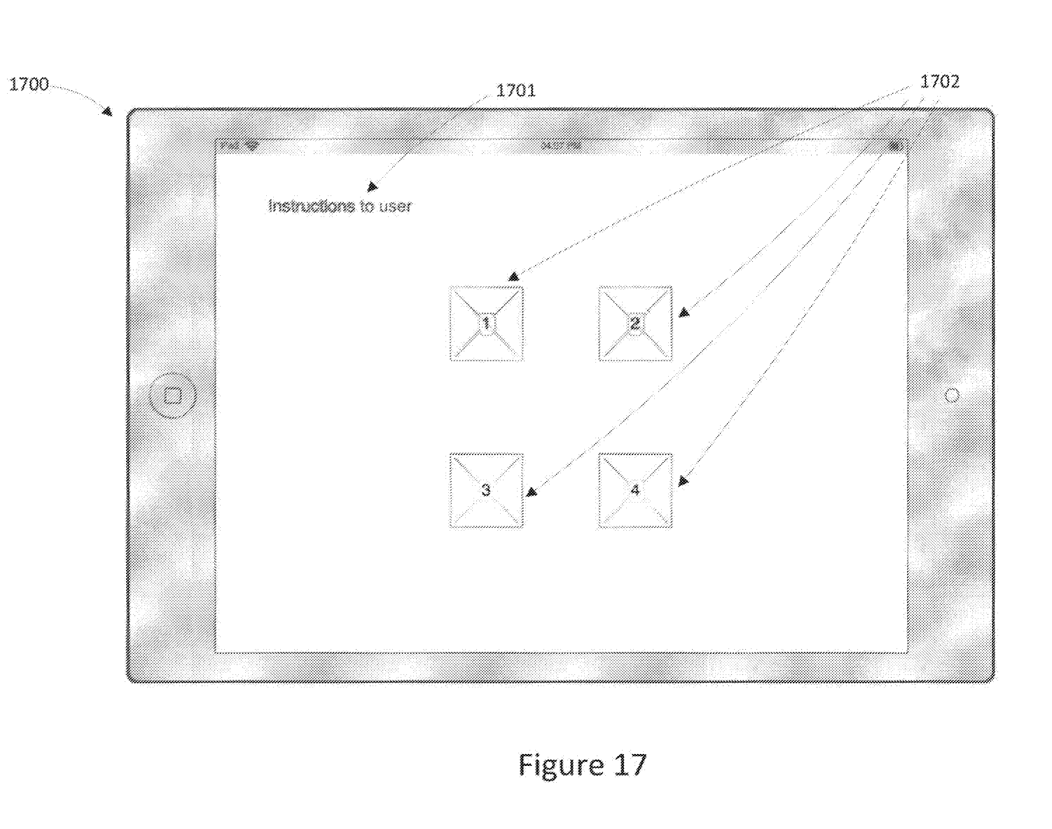

An example screen layout 1700 for the “Odd One” game is shown in FIG. 17. Screen layout 1700 includes instructions 1701, and an array of pictures 1702. Instructions 1701 may prompt a user to select a picture from array 1702 that is the odd picture from the group. The pictures in array 1702 may be photos of human faces in some embodiments.

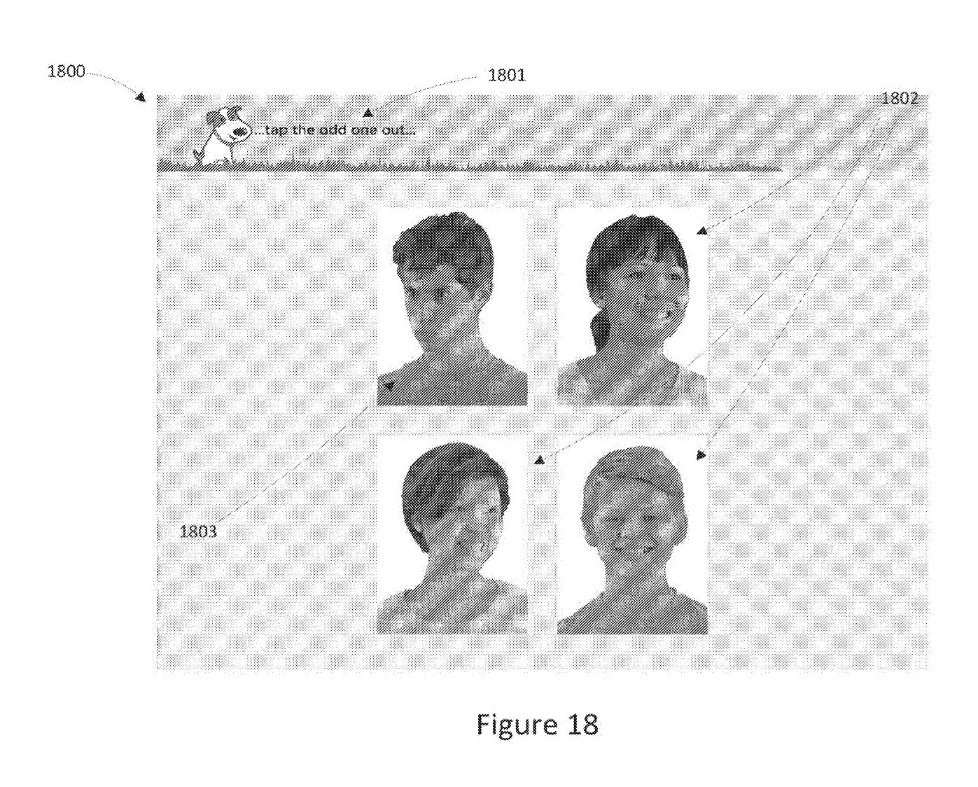

An example screenshot 1800 for the “Odd One” game is shown in FIG. 18. Screenshot 1800 includes instructions 1801, and an array of pictures 1802, 1803. In the illustrated embodiments, instructions 1801 prompt a user to “tap the odd one out”. In the illustrated embodiment, the pictures in array 1802, 1803 are pictures of human faces showing a variety of emotions. Image 1803 is showing a picture of a person showing an angry emotion, while images 1802 show a happy emotion.

An example of a basic communication type game for determining a measure of the user's ability to comprehend social intent is represented by FIG. 19. FIG. 19 is a process flow diagram of a computer-implemented method 1900 of determining a measure of a user's ability to comprehend social intent, according to some embodiments, the method 1900 operable by the assessment system 100, 302, and in particular, execution of the processor(s) 102, 312 of an interactive game component 132, 346 of the games module 124, 338 of the assessment application 118, 330. In this example, the basic communication type game is referred to as SOCIAL SCENES.

Method 1900 starts when a user begins the “Social Scenes” game, at 1901, for example, by selecting selectable option 605 on the assessment platform. In some embodiments, processor 102, 312 executes an interactive game component 132, 346 that causes a set of instructions to be shown to the user at 1902, which may include an instruction such as “tap the odd one out”. Processor 102, 312 causes an array of image stimuli to be to be displayed on the assessment platform shown on a screen of the user interface 106, 324, which may include images of a number of social scenes, at 1903. In some embodiments, one of the image stimulus will depict one form of social interaction, and the remaining photo stimuli will depict a second, different form of social interaction. The user selects a image that they believe to be the odd one out, i.e., the image stimulus depicting a social interaction different from that of the other image stimuli of the array, 1904. Processor 102, 312 determines the round or trial outcome based on a comparison of the selected image stimulus with the odd image stimulus, i.e., the image depicting a social interaction different from that of the other image stimuli of the array, 1905. For example, if the selected image stimulus corresponds with the odd image stimulus, the trial outcome comprises an indication that the input provided by the user was correct and if the selected image stimulus does not correspond with the odd image stimulus, the trial outcome comprises an indication that the input provided was incorrect. In some embodiments, the trial outcome may further comprise the selected image stimulus and the odd image stimulus, which may be employed by the assessment module 128, 342 or other component of the assessment application 118, 330 or assessment system in diagnosing the social competency of the user. For example, such information may provide an indication that a user has difficulties in distinguishing between two or more specific social interactions in particular. Processor 102, 312 checks to see whether all trials are complete, at 1906. If more trials are to be performed, a new array of image stimuli are generated and provided to the assessment platform, at 1903. Once all trials are complete, the games module 124, 338 determines the game outcome based on the trial outcome(s), at 1907. The method moves to 1908, where the Social Scenes game is considered to be complete. Processor 102, 312 may cause the Social Scenes game to exit at 1908.

The Social Scenes game assesses social intent processing over 1 or more trials, which may be 2, 3, 4, 5, 6, or more trials. In some embodiments, the Social Scenes game may have 12 trials. Each trial may cause a number of images to be displayed on the screen displaying social scenarios. In some embodiments, 2, 3, 4, 5, 6, 7, 8 or more images may be displayed. The images may be displayed in a grid, which may be a two by two grid in some embodiments, or they may be arranged in a different configuration on the screen. All but one of the images may show one social scenario, while one image may show a second social scenario. The user may be asked to select the odd scenario. The Social Scenes game may take approximately five minutes to complete. In some embodiments, the game may take approximately two, three, four, six, seven, eight, nine, ten, or more minutes to complete.

The game outcome or measure of a user's ability to comprehend social intent may be determined based on a number of measured results of the Social Scenes game. For example, the total number of items completed, the percentage of the available items completed, the number of correct answers, the response time for each individual item, the total time taken to complete the game, and the user's verbal explanation may be measured and used to determine a measure of information processing speed of a user.

In some embodiments, the Social Scenes game may include a practice round, to allow the user to become familiar with the format of the game. During the practice round, an assessor may explain the Social Scenes game to the user, telling the user to tap the odd one out. The assessor may explain that they don't want to user to tap the image that is the odd one out because of the colours in the image, the number of people in the image, or the ages of the people. In some embodiments, the assessor may play one round of the game as a demonstration to the user. In some embodiments, the assessor may also ask the user to try it. In some embodiments, the practice round may consist of 3 trials. In some other embodiments, the practice round may have 1, 2, 4, 5, or more trials. Once the practice round is complete, the assessed phase of the Social Scenes game may begin, as described above with reference to FIG. 19.

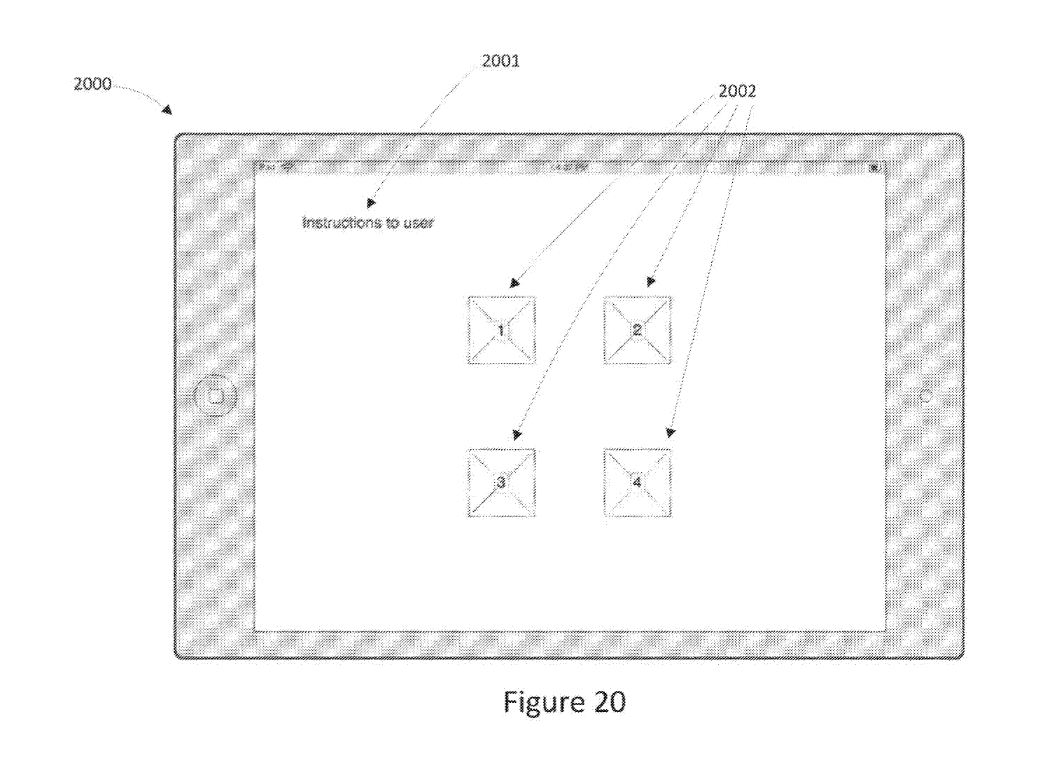

An example screen layout 2000 for the “Social Scenes” game is shown in FIG. 20. Screen layout 2000 includes instructions 2001, and an array of pictures 2002. Instructions 2001 may prompt a user to select a picture from array 2002 that is the odd picture from the group. The pictures in array 2002 may be photos of social scenes in some embodiments.

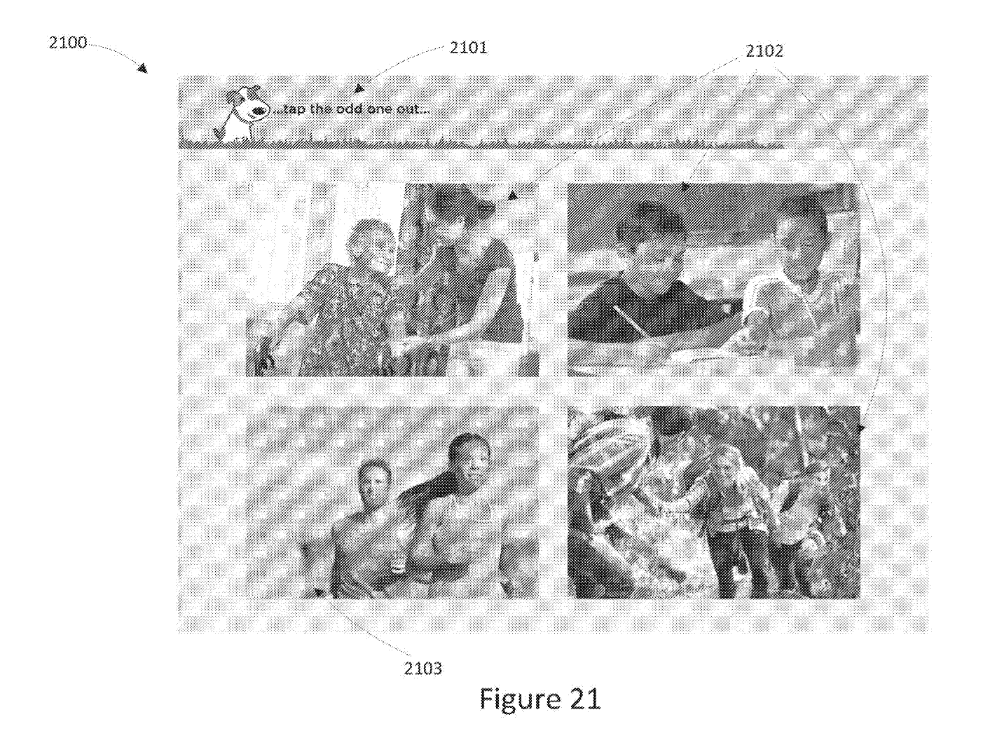

An example screenshot 2100 for the “Social Scenes” game is shown in FIG. 21. Screenshot 2100 includes instructions 2101, and an array of pictures 2102, 2103. In the illustrated embodiments, instructions 2101 prompt a user to “tap the odd one out”. In the illustrated embodiment, the pictures in array 2102, 2103 are pictures of social scenes showing a variety of social interactions. Image 2103 is showing a picture of a competitive social interaction, while images 2102 show a supportive social interaction.

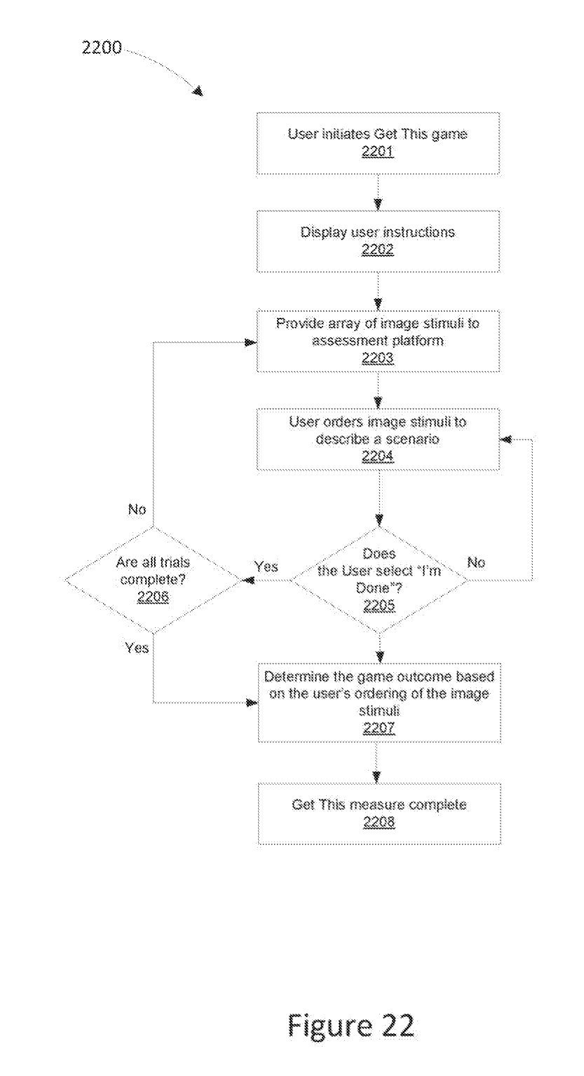

An example of a complex communication type game for determining a measure of pragmatics, such as language use in social context and/or social intention, is represented by FIG. 22. FIG. 22 is a process flow diagram of a computer-implemented method 2200 of determining a measure of pragmatics, according to some embodiments, the method 2200 operable by the assessment system 100, 302, and in particular, execution of the processor(s) 102, 312 of an interactive game component 132, 346 of the games module 124, 338 of the assessment application 118, 330. In this example, the complex communication type game is referred to as GET THIS.

Method 2200 starts when a user initiates the “Get This” game, for example, by selecting selectable option 605 on the assessment platform, at 2201. In some embodiments, processor 102, 312 executes an interactive game component 132, 346 that causes a set of instructions to be shown to the user, at 2202, which may include an instruction such as “move the pictures around so that they are in the correct order”. Processor 102, 312 causes an array of image stimuli to be provided to the assessment platform and shown on a screen of the user interface 106, 324, at 2203. The image stimuli each depict a scene of a scenario or story and when placed in order, collectively depict the scenario or story playing out, but which are shown on the screen in an arbitrary or apparently random order. The image stimuli may comprise illustrated stimuli and/or photo stimuli. The user reorders the image stimuli into an order that they believe describes a scenario or story. Processor 102, 312 monitors or checks whether the user has selected an “I'm Done” button to show that they have made their selection, at 2205. If not, the method goes back to 2204. If the user does select the “I'm Done” button, processor 102, 312 checks to see whether all trials are complete at step 2206. For example, in some embodiments, the “Get This” game may comprise a plurality of rounds or trials. If more trials are to be performed, a new set of image stimuli are generated and provided to the assessment platform at 2203. Once all trials are complete, the games module 124, 338 determines the game outcome based on the ordering of the image stimuli input by the user, at 2207. For example, for each trial, the games module 124, 338 compares the user's ordering of the images with a target ordering of the images to determine whether the user has correctly ordered image stimuli. In some embodiments, the games module 124, 338 may be further configured to monitor a length of time taken by the user to order the image stimuli and the game outcome may be further based on time taken to order the image stimuli. The method then moves to 2208, where the Get This game is considered to be complete. Processor 102, 312 may cause the Get This game to exit at step 2208.

The Get This game assesses social interpretation across 1 or more trials, which may be 2, 3, 4, 5, 6, or more trials. In some embodiments, the Get This game may have 12 trials. The Get This game may cause a number of illustrated images to appear in a random order on the screen, and asked to move them into an order that makes sense given the social scenario. In some embodiments, the Get This game may cause 1, 2, 3, 4, 5, 6, 7, 8, 9, 10 or more images to be shown on the screen.

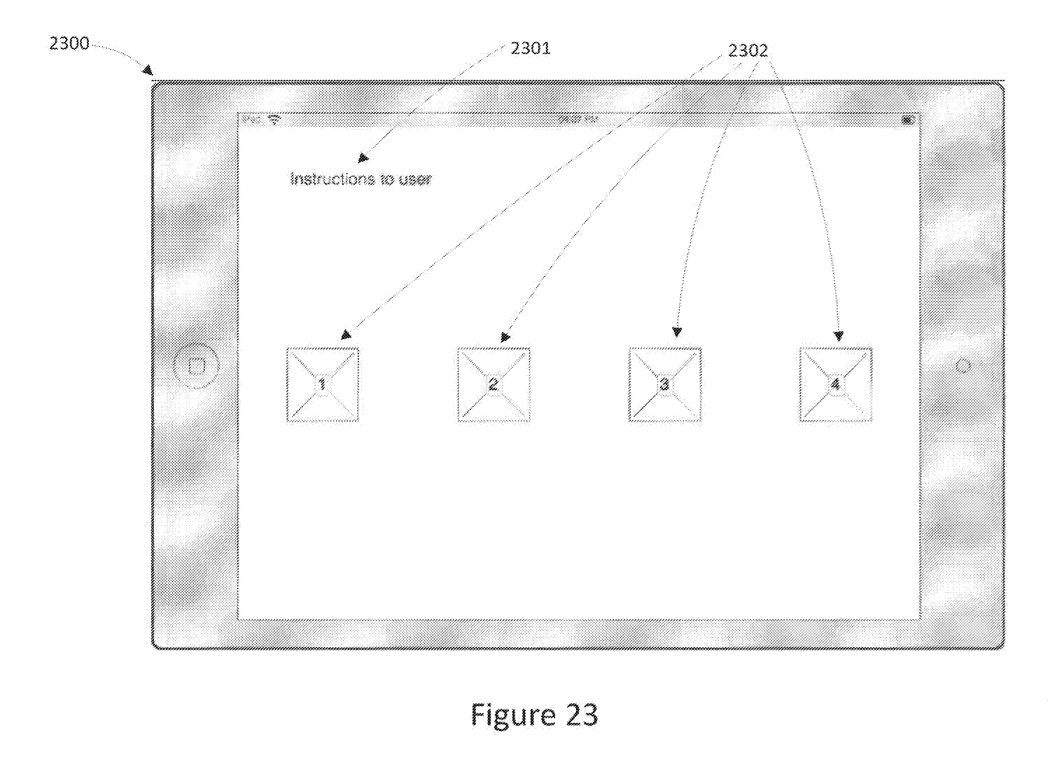

An example screen layout 2300 for the “Get This” game is shown in FIG. 23. Screen layout 2300 includes instructions 2301, and an array of images 2302. Instructions 2301 may prompt a user to order the images in array 2302 so that they are in the correct order. Images 2302 may be illustrations of a scenario or scene in some embodiments.

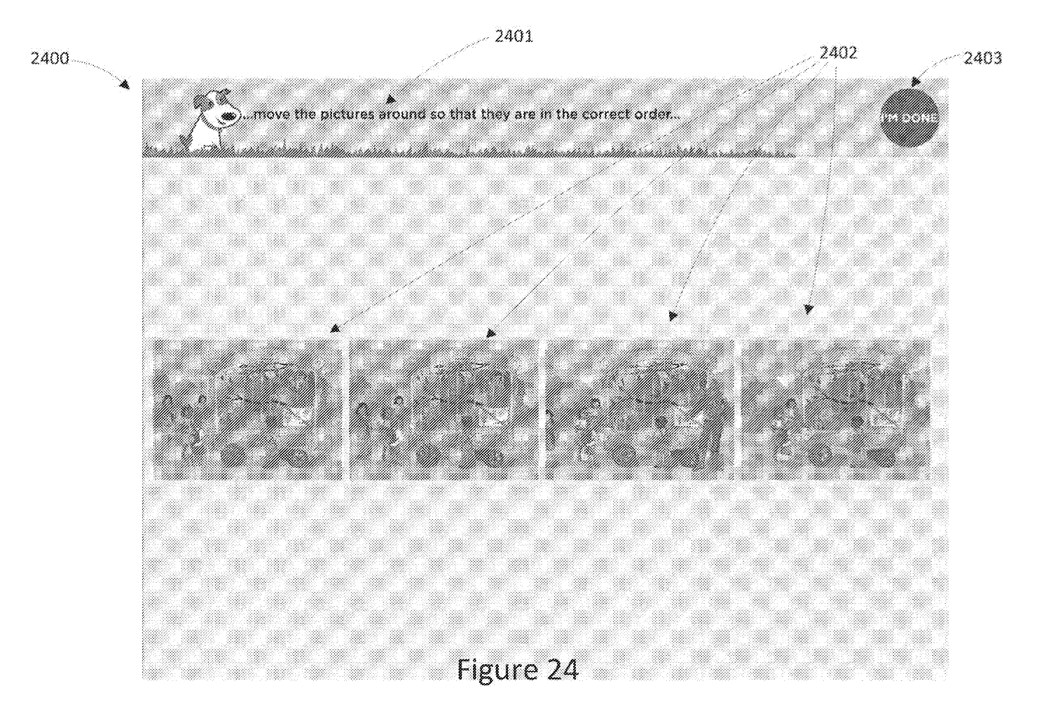

An example screenshot 2400 for the “Get This” game is shown in FIG. 24. Screenshot 2400 includes instructions 2401, an array of images 2402, and an “I'm done” button 2403. In the illustrated embodiments, instructions 2401 prompt a user to “move the pictures around so that they are in the correct order”. In the illustrated embodiment, the pictures in array 2402 are illustrations of a screen showing a boy attempting to feed a monkey, and being shown a sign that stipulates that the monkeys are not to be fed. Once a user has ordered the images, they may be able to indicate this by pressing the “I'm done” button 2403.

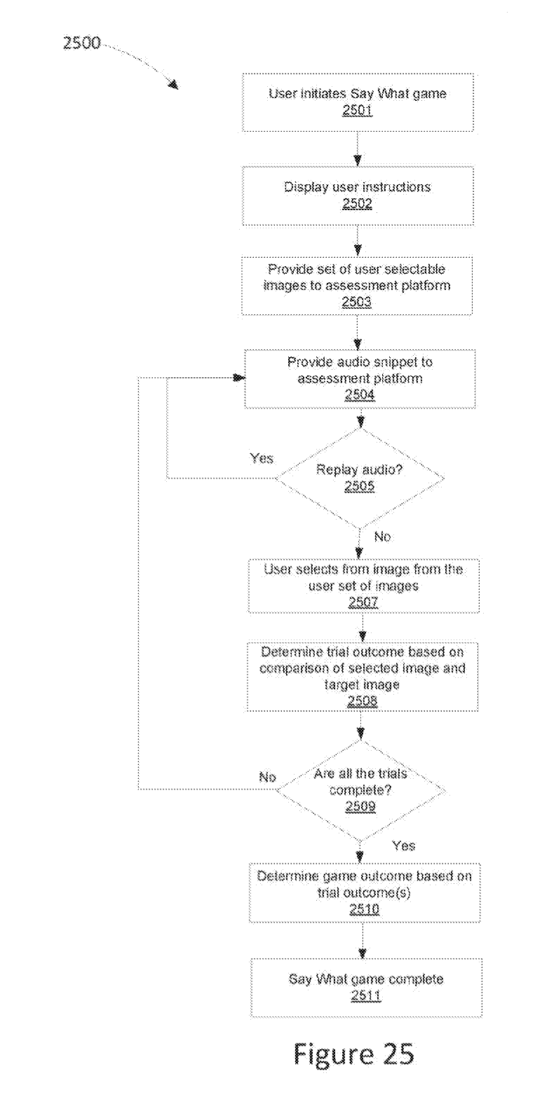

An example of a complex communication type game for determining a measure of prosody, such as use and understanding of irony and/or empathy, is represented by FIG. 25. FIG. 25 is a process flow diagram of a computer-implemented method 2500 of determining a measure of prosody according to some embodiments, the method 2500 operable by the assessment system 100, 302, and in particular, execution of the processor(s) 102, 312 of an interactive game component 132, 346 of the games module 124, 338 of the assessment application 118, 330. In this example, the complex communication type game is referred to as SAY WHAT.

Method 2500 starts when a user initiates the “Say What” game, at 2501, for example, by selecting selectable option 605 on the assessment platform. In some embodiments, processor 102, 312 executes an interactive game component 132, 346 that causes a set of instructions to be shown to the user at 2502, which may include an instruction such as “tap the correct emotion”. The interactive game component 132, 346 causes a set of user selectable images, each corresponding to a different emotion, to be provided to the assessment platform and shown on a screen of the user interface 106, 324, at 2503. The interactive game component 132, 346 causes an audio snippet corresponding to an emotion to be provided to the assessment platform and played to the user via the user interface 106, 324, at 2504. In some embodiments, the audio snippet is replayed. In some embodiments, the interactive game component 132, 346 provides an option to the assessment platform to allow the user to select to have the audio snippet replayed, at 2505. If the user elects to have the audio snippet replayed the method reverts to 2504. If the user elects not to have the audio snippet replayed, the user continues with the game by selecting an image from the set of images displayed which depicts an the emotion that the user considers corresponds to the emotion evoked or suggested by the audio snippet, at 2507. Processor 102, 312 then checks to see whether all trials are complete at 2509. The interactive game component 132, 346 of the games module 124, 338 determines the trial outcome based on a comparison of the selected image with a target image associated with the audio snippet. For example, for each trial, the interactive game component 132, 346 compares the selected image with a target image to determine whether the user has correctly identified the emotion evoked or suggested by the audio snippet. In some embodiments, the interactive game component 132, 346 may be further configured to monitor a length of time taken by the user to select the image from the set of images and the trial outcome may be further based on time taken to select the image. If more trials are to be performed, a new audio snippet is provided to the assessment platform, at step 2503. Once all trials are complete, the games module 124, 338 determines the game outcome based on the trial outcome(s), at 2510. The method moves to 2511, where the Say What game is considered to be complete. Processor 102, 312 may cause the Say What game to exit at step 2511.

The Say What game assesses prosody recognition across 1 or more trails, which may be 2, 3, 4, 5, 6, or more trials. In some embodiments, the Get This game may have 10 trials. The user may be played a short audio recording of a content neutral sentence such as “Vicki drove her car to work on Tuesday”, spoken with an emotional tone. The user may be asked to identify the tone from a list of emotions. In some embodiments, the list may contain 2, 3, 4, 5, 6, 7, 8, 9, 10, or more emotions to choose from.

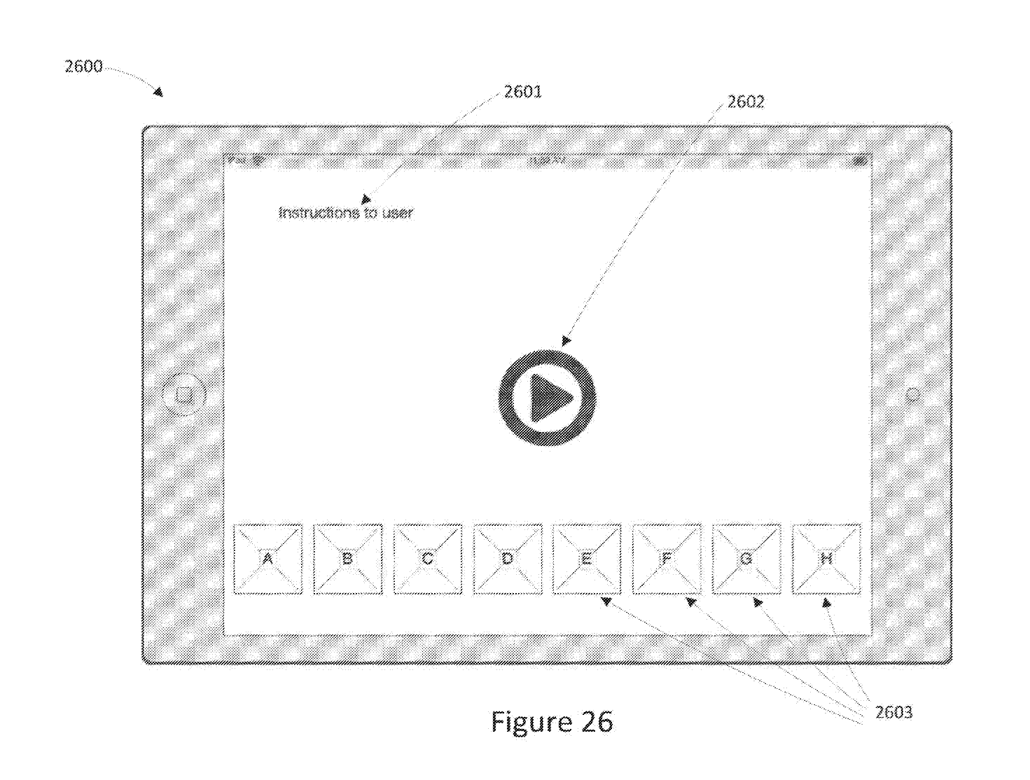

An example screen layout 2600 for the “Say What” game is shown in FIG. 26. Screen layout 2600 includes instructions 2601, an audio play button 2602, and an array of pictures 2603 that depict different emotions. Instructions 2601 may prompt a user to select a picture from array 2603 that matches the emotion played in the audio.

Social cognition type games or tests are designed to determine a measure of the user's comprehension of communication such as emotional perception, intent attribution, empathy, perspective taking, theory of mind and/or moral reasoning. For example, a basic social cognition type game may comprise a game configured to determine a measure of the user's ability to recognise emotion and/or to perceive emotion in themselves and/or others. A more complex social cognition type game may comprise a game configured to determine a measure of attribution of social intent, social information processing, socio-moral solutions, perspective taking/empathy, moral reasoning and/or theory of mind, for example.

Emotion recognition is important for recognising and identifying emotional states. As with emotion perception, recognising and identifying emotions is necessary for appropriate response to social interactions.

Emotion perception involves distinguishing between emotional states reflected in facial expression. Facial expression directly influences one's response and reciprocal interaction. Misperception of facial expression may result in inappropriate responses to social situations.

Moral reasoning is important for perspective taking and decision making, allowing an individual to engage in social interactions with empathy, and use knowledge of right and wrong to understand and negotiate their social environment. Previous research indicates that impairments in moral reasoning may result in socially unacceptable behaviour.

Theory of mind is the ability to understand and differentiate between one's own and others mental states. Impairment in theory of mind interferes with understanding and engaging in complex social behaviour and development of prosocial behaviour.

An example of a basic social cognition type game for determining a measure of the user's ability to recognise emotion is represented by FIG. 27. FIG. 27 is a process flow diagram of a computer-implemented method 2700 of determining a measure of the user's ability to recognise emotion, according to some embodiments, the method 2700 operable by the assessment system 100, 302, and in particular, execution of the processor(s) 102, 312 of an interactive game component 132, 346 of the games module 124, 338 of the assessment application 118, 330. In this example, the basic social cognition type game is referred to as FINDING EMO(tion).

Method 2700 starts when a user initiates the “Finding Emo” game, by selecting selectable option 605 on the assessment platform, at 2701. In some embodiments, processor 102, 312 executes an interactive game component 132, 346 that causes a set of instructions to be shown to the user on the assessment platform, at 2702, which may include an instruction such as “tap the odd one out”. Processor 102, 312 causes an array of image stimuli to be provided to the assessment platform and shown on a screen of the user interface 106, 324, at 2703. The image stimuli may include images or photos of full body shots of a number of people expressing different emotions. In some embodiments, one of the image stimuli will be showing one emotion, and the remaining image stimuli will be showing a second, different emotion. The user selects a photo stimulus that they believe to be the odd one out, at 2704. Processor 102, 312 determines a round or trial outcome based on a comparison of the selected image stimulus with the odd image stimulus, i.e., the image stimulus depicting an emotion different from that of the other image stimuli of the array, at 2705. For example, if the selected image stimulus corresponds with the odd image stimulus, the trial outcome comprises an indication that the input provided was correct and if the selected image stimulus does not correspond with the odd image stimulus, the trial outcome comprises an indication that the input provided was incorrect. In some embodiments, the trial outcome may further comprise the selected image stimulus and the odd image stimulus, which may be employed by the assessment module 128, 342 or other component of the assessment application 118, 330 or assessment system in diagnosing the social competency of the user. For example, such information may provide an indication that a user has difficulties in distinguishing between two or more specific emotions in particular. Processor 102, 312 checks to see whether all trials are complete, at 2706. If more trials are to be performed, a new set of image stimuli are generated and provided to the assessment platform, at 2703. Once all trials are complete, the games module 124, 338 determines the game outcome based on the trial outcome(s), at 2707. The method then moves to step 2708, where the Finding Emo measure is considered to be complete. Processor 102, 312 may cause the Finding Emo game to exit at step 2708.

The Finding Emo game assesses emotion perception over 1 or more trials, which may be 2, 3, 4, 5, 6, or more trials. In some embodiments, the Finding Emo game may have 22 trials. Each trial may cause a number of preselected images to be displayed. In some embodiments, 2, 3, 4, 5, 6, 7, 8 or more images may be displayed. The images may be displayed in a grid, which may be a two by two grid in some embodiments, or they may be arranged in a different configuration on the screen. All but one of the images may show people displaying one emotion, while one image may show a person displaying a second emotion. Each image may display an image of a person from the shoulder up and facing the user. They may be facing the user straight on, or at a left or right angle. The person in the image may appear on a white background. The user may be asked to select the image displaying the odd emotion in each trial. The Finding Emo game may have a number of easy trials and a number of hard trials. In some embodiments, the game may have an equal number of easy and hard trials. The Finding Emo game may take approximately five minutes to complete. In some embodiments, the game may take approximately two, three, four, six, seven, eight, nine, ten, or more minutes to complete.

The game outcome or measure of emotion perception may be determined based on a number of measured results of the Finding Emo game. For example, the total number of items completed, the percentage of the available items completed, the number of correct answers, the response time for each individual item, the total time taken to complete the game, and the user's verbal explanation may be measured and used to determine a measure of information processing speed of a user.

In some embodiments, the Finding Emo game may include a practice round, to allow the user to become familiar with the format of the game. During the practice round, an assessor may explain the Finding Emo game to the user, telling the user to tap the odd one out. In some embodiments, the assessor may play one round of the game as a demonstration to the user. The assessor may then ask the user to try it. In some embodiments, the practice round may consist of three trials. In some other embodiments, the practice round may have 1, 2, 4, 5, or more trials. Once the practice round is complete, the assessed phase of the Finding Emo game may begin, as described above with reference to FIG. 27.

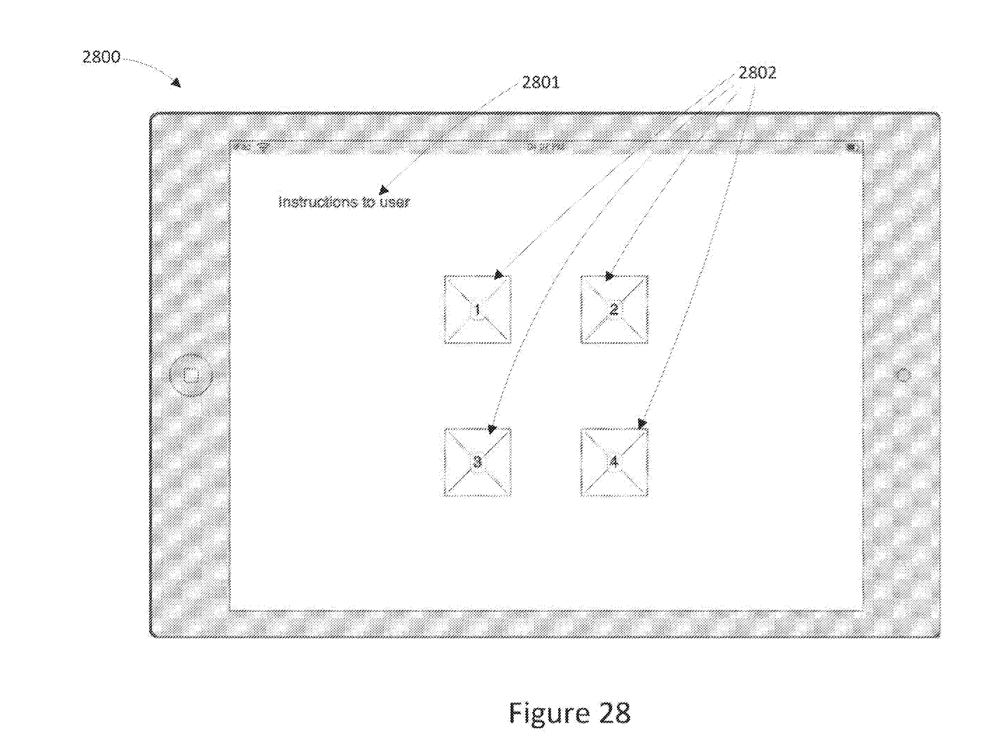

An example screen layout 2800 for the “Finding Emo” game is shown in FIG. 28. Screen layout 2800 includes instructions 2801, and an array of pictures 2802. Instructions 2801 may prompt a user to select a picture from array 2802 that is the odd picture from the group. The pictures in array 2802 may be photos of people expressing emotion through body language, in some embodiments.

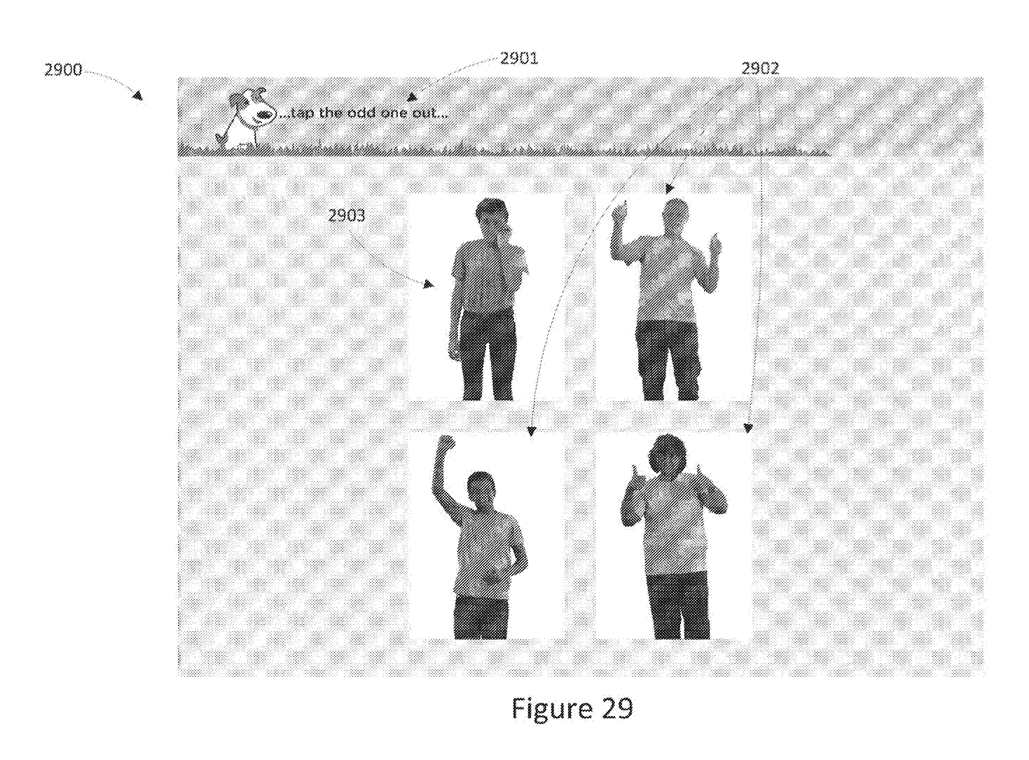

An example screenshot 2900 for the “Finding Emo” game is shown in FIG. 29. Screenshot 2900 includes instructions 2901, and an array of pictures 2902, 2903. In the illustrated embodiment, instructions 2901 prompt a user to “tap the odd one out”. In the illustrated embodiment, the pictures in array 2902, 2903 are pictures of people expressing different emotions through their body language. Image 2903 is showing a picture of a sad emotion, while images 2902 show a picture of a happy emotion.

An example of a basic social cognition type game for determining a measure of a user's emotion perception of self and others is represented by FIG. 30. FIG. 30 is a process flow diagram of a computer-implemented method 3000 of determining a measure of a user's emotion perception of self and others, according to some embodiments, the method 3000 operable by the assessment system 100, 302, and in particular, execution of the processor(s) 102, 312 of an interactive game component 132, 346 of the games module 124, 338 of the assessment application 118, 330. In this example, the basic social cognition type game is referred to as MATCHING EMO(tion).

Method 3000 starts when a user initiates the “Matching Emo” game, at 3001, for example, by selecting selectable option 605 on the assessment platform. In some embodiments, processor 102, 312 executes an interactive game component 132, 346 that causes a set of instructions to be shown to the user on the assessment platform, at 3002, which may include an instruction such as “tap the face that matches the word”. Processor 102, 312 causes an array of emotion image stimuli to be provided to the assessment platform shown on a screen of the user interface 106, 324, at 3003. For example, the emotion image stimuli may include a number of photos or cartoon faces displaying a range of emotions. An emotion label is provided to the assessment platform and displayed on the screen of the user interface 106, 324, at 3004. The user selects the emotion stimulus that they believe to match the emotion label, at 3005, and in some embodiments, the assessment platform shows the label moving toward and/or at or beside the selected emotion image stimulus, at 3006. Processor 102, 312 checks whether a target label associated with the selected image stimulus matches or corresponds substantially with the emotion label, at 3007. If not, the emotion label returns or “bounces back” to it's home position on the assessment platform, at 3008 and the user is prompted to attempt a new selection, at 3005. If the target label associated with the selected image stimulus does match or substantially correspond with the emotion label, the method moves to 3009, and processor 102, 312 determines a round or trial outcome based on a time value indicative of the time taken by the user to correctly associate the emotion label with the image stimulus, at 3009. In some embodiments, the games module may record incorrect attempts made by the user, which may be employed by the assessment module 128, 342 or other component of the assessment application 118, 330 or assessment system in diagnosing the social competency of the user. For example, such information may provide an indication that a user has difficulties in distinguishing between two or more specific emotions in particular. Processor 102, 312 checks to see whether all trials are complete, at 3010. If more trials are to be performed, a new set of emotion image stimuli are generated and provided to the assessment platform, at 3003. Once all trials are complete, the games module 124, 338 determines the game outcome based on the trial outcome(s), at 3011. The method then moves to step 3012, where the Matching Emo game is considered to be complete. Processor 102, 312 may cause the Matching Emo game to exit at step 3012.

The Matching Emo game assesses emotion recognition over 1 or more trials, which may be 2, 3, 4, 5, 6, or more trials. In some embodiments, the Matching Emo game may have 24 trials. Each trial may cause a number of illustrated faces to be displayed. In some embodiments, 2, 3, 4, 5, 6, 7, 8 or more faces may be displayed. The faces may be displayed in a grid, which may be a four by two grid in some embodiments, or they may be arranged in a different configuration on the screen. Each face may display one of a number of predetermined emotions. There may be 3, 4, 5, 6, 7, 8, 9, 10, 11, 12, or more predetermined emotions in some embodiments. Some emotions may include worried, unsure, bored, angry, disgusted, shy, surprised, sad, questioning, loving, scared, annoyed and happy, for example. The name of a target emotion may be displayed at the bottom of the screen. The user may be asked to select the illustration that matches the target emotion. The target emotion may be read aloud by the assessor if the user does not have adequate reading ability. If the user makes an incorrect response, the user may be asked to make another selection. The Matching Emo game may take approximately eight minutes to complete. In some embodiments, the game may take approximately two, three, four, five, six, seven, nine, ten, or more minutes to complete.

The game outcome or measure of emotion recognition may be determined based on a number of measured results of the Matching Emo game. For example, the total number of items completed, the percentage of the available items completed, the number of correct answers, the response time for each individual item, and the total time taken to complete the game may be measured and used to determine a measure of information processing speed of a user.

In some embodiments, the Matching Emo game may include a practice round, to allow the user to become familiar with the format of the game. During the practice round, an assessor may explain the Matching Emo game to the user, telling the user to tap the face that matches the word. In some embodiments, the assessor may play one round of the game as a demonstration to the user. The assessor may then ask the user to try it. In some embodiments, the practice round may consist of three trials. In some other embodiments, the practice round may have 1, 2, 4, 5, or more trials. Once the practice round is complete, the assessed phase of the Finding Emo game may begin, as described above with reference to FIG. 30.

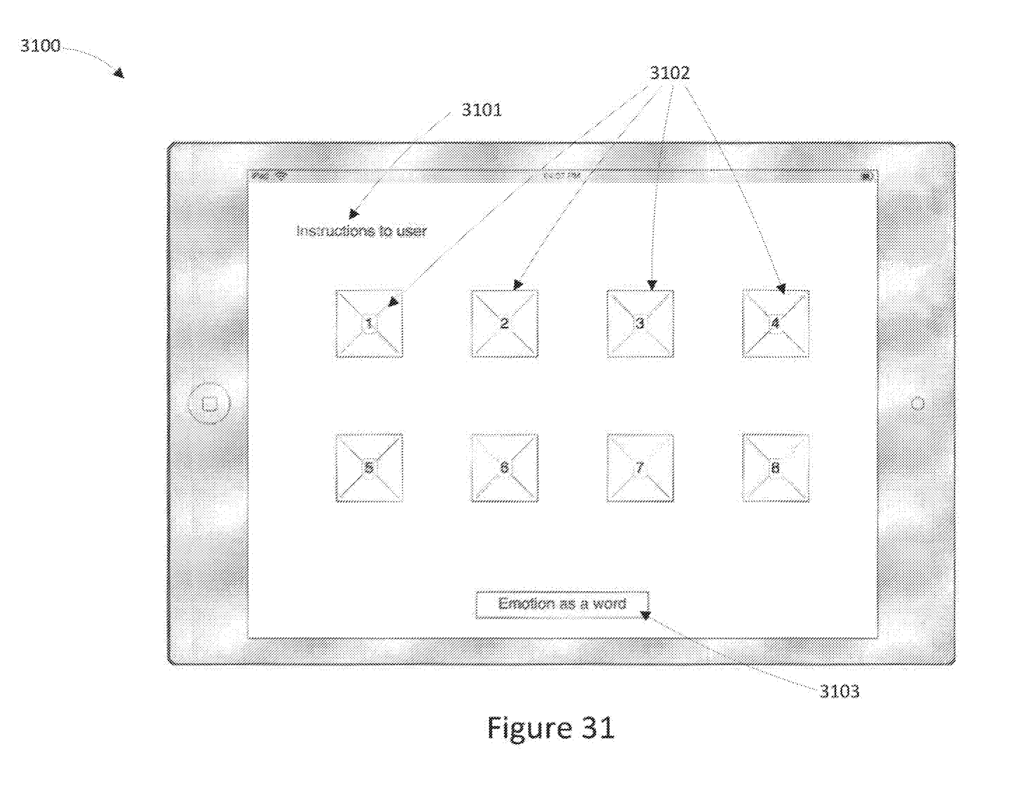

An example screen layout 3100 for the “Matching Emo” game is shown in FIG. 31. Screen layout 3100 includes instructions 3101, an array of images 3102, and a label 3103. Label 3103 may be configured to display an emotion as a word. Instructions 3101 may prompt a user to select an image from array 3102 that matches word displayed by label 3103. The images in array 3102 may be cartoon images depicting human faces in some embodiments.

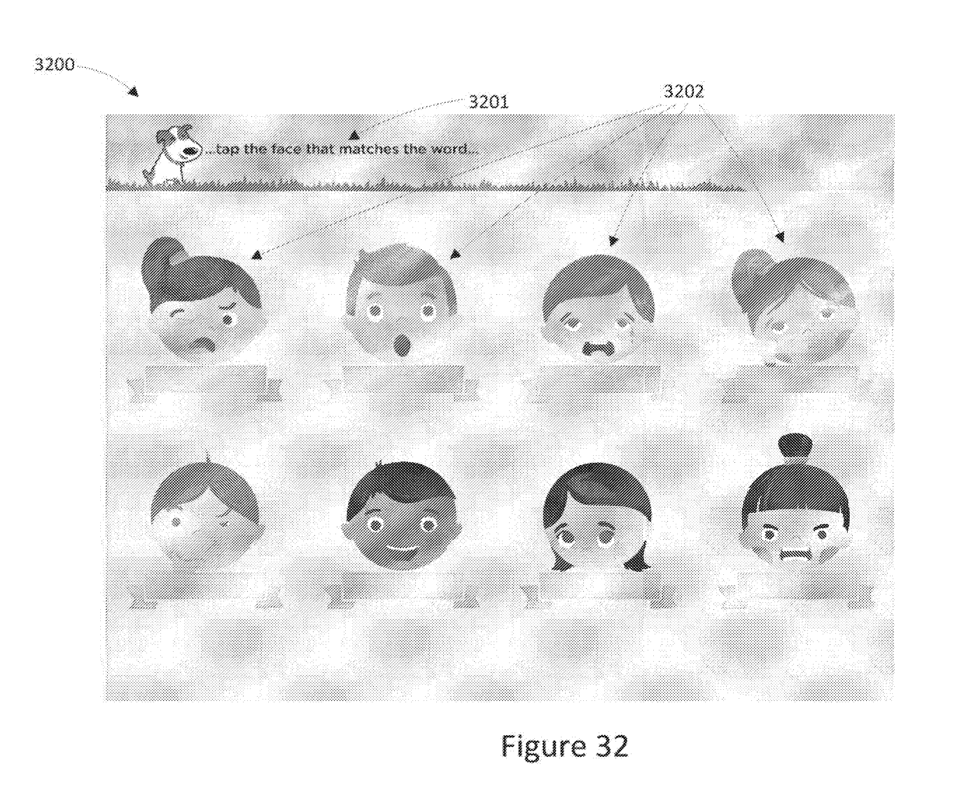

An example screenshot 3200 for the “Matching Emo” game is shown in FIG. 32. Screenshot 3200 includes instructions 3201, and an array of images 3202. In the illustrated embodiments, instructions 3201 prompt a user to “tap the face that matches the word”. In the illustrated embodiment, the images in array 3202 are cartoon images of human faces showing a variety of emotions.

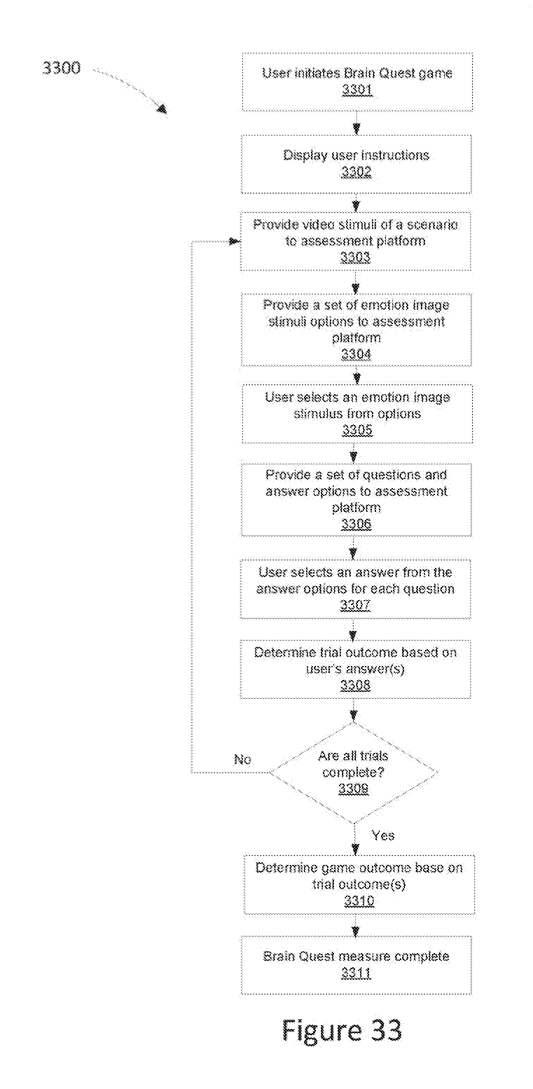

An example of a complex social cognition type game for determining a measure of attribution of social intent and/or social information processing of the user is represented by FIG. 33. FIG. 33 is a process flow diagram of a computer-implemented method 3300 of determining a measure of attribution of social intent and/or social information processing, according to some embodiments, the method 3300 operable by the assessment system 100, 302, and in particular, execution of the processor(s) 102, 312 of an interactive game component 132, 346 of the games module 124, 338 of the assessment application 118, 330. In this example, the complex social cognition type game is referred to as BRAIN QUEST.

Method 3300 starts when a user initiates the “Brain Quest” game, at 3301, for example, by selecting selectable option 605 on the assessment platform. In some embodiments, processor 102, 312 executes an interactive game component 132, 346 that causes a set of instructions to be shown to the user on the assessment platform, at 3302, which may include an instruction such as “tap the emotion that matches the video”. Processor 102, 312 causes a video stimuli to be provided to the assessment platform and to play on a screen of the user interface 106, 324, at 3304. Processor 102, 312 also causes a set of user selectable images, each corresponding to a different emotion, to be provided to the assessment platform and shown on a screen of the user interface 106, 324, at 3305. For example, the user selectable images may comprise emotion image stimuli. The user selects an emotion image stimulus from the set of user selectable images that they believe to match the emotion being suggested or evoked in the video stimuli, at 3305. Processor 102, 312 also causes one or more questions and for each questions, an associated set of answer options, to be provided to the assessment platform. The questions relate to the video stimuli and in particular in some embodiments, to a social intent suggested by the video stimuli. For each of the questions, the user selects an answer from the associated answer options provided using the user interface 106, 324, at 3307. The interactive game component 132, 346 of the games module 124, 338 determines a trial outcome based on whether or not the user selected the correct emotion image stimulus associated with the video stimulus and a number of correct answers to the question(s) provided by the user. For example, the interactive game component 132, 346 may be configured to compare the selected emotion image stimulus with a target emotion image stimulus for the video stimulus and the interactive game component 132, 346 may be configured to compare the answer selected by the user for a particular question, with a target answer associated with the question. If the selected answer corresponds substantially with the target answer, the user is deemed to have correctly answered the question and if the selected answer does not correspond substantially with the target answer, the user is deemed to have incorrectly answered the question. In some embodiments, the trial outcome may be further based on a time value indicative of the time taken by the user to correctly answer the questions of the trial. Processor 102, 312 checks to see whether all trials are complete, at 3309. If more trials are to be performed, a new video stimuli is provided to the assessment platform and played to the user, at 3303. Once all trials are complete, the games module 124, 338 determines the game outcome based on the trial outcome(s), at 3310. The method then moves to 3311, where the Brain Quest game is considered to be complete. Processor 102, 312 may cause the Brain Quest game to exit at step 3311.

The Brain Quest game assesses social-cognitive functioning across 1 or more trials, which may be 2, 3, 4, 5, 6, or more trials. In some embodiments, the Brain Quest game may have 10 trials. The trials are individualised based on the user's age and gender so that the stimuli closely resemble a real-life scenario for the user. The Brain Quest game may cause a short video to be played that shows a negative social interaction. The user is then asked about their reaction to the video. For example, the user may be asked, “What would you do?” with multiple choice options relevant to the scenario displayed on the screen. Alternatively or in addition, the user may be asked “How would you feel?” and told to select an emotion from a list that best describes how they would feel in that social interaction.

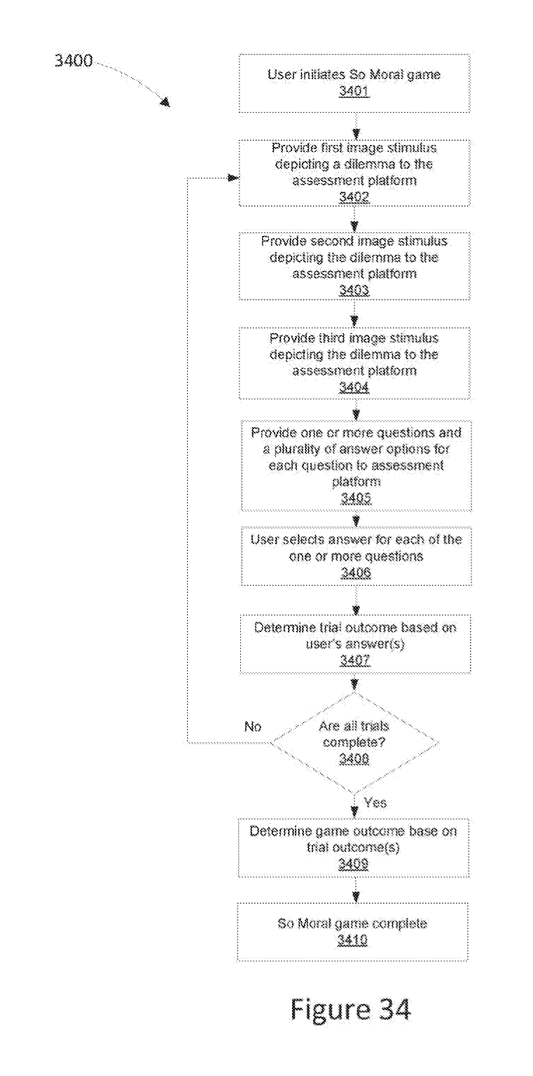

An example of a complex social cognition type game for determining a measure of socio-moral solutions, perspective taking/empathy, and/or moral reasoning of the user is represented by FIG. 34. FIG. 34 is a process flow diagram of a computer-implemented method 3400 of determining a measure of socio-moral solutions, perspective taking/empathy, and/or moral reasoning, according to some embodiments, the method operable by the assessment system 100, 302, and in particular, execution of the processor(s) 102, 312 of an interactive game component 132, 346 of the games module 124, 338 of the assessment application 118, 330. In this example, the complex c social cognition type game is referred to as SO-MORAL.

Method 3400 starts when a user initiates the “Multiple Morals” game, at 3401, for example, by selecting selectable option 605 on the assessment platform. In some embodiments, processor 102, 312 executes an interactive game component 132, 346 that causes a set of instructions to be shown to the user on the assessment platform. Processor 102, 312, causes a first image stimulus depicting a dilemma to be provided to the assessment platform shown to the user on a display of the of the user interface 106, 324, at 3402. In some embodiments, processor 102, 312, causes a second image stimulus depicting the dilemma to be provided to the assessment platform shown to the user on a display of the of the user interface 106, 324, at 3403. In some embodiments, processor 102, 312, causes a third image stimulus depicting the dilemma to be provided to the assessment platform shown to the user on a display of the of the user interface 106, 324, at 3404. In some embodiments, the first, second and third image stimuli are presented on the assessment platform as an array. Processor 102, 312 provides one or more questions and a set of answer options for each of the one or more questions to the assessment platform, at 3405. For each of the questions, the user selects an answer from the associated answer options provided using the user interface 106, 324, at 3406. In some embodiments, processor provides a first question asking the user what they would do in the situation or dilemma depicted in the image stimuli and the answer options for the first question provide a plurality of different actions that might be taken in view of the dilemma. In some embodiments, processor provides a second question asking the user why they selected the selected answer option in response to the first question and the answer options for the second question provide a plurality of different reasons to account for why the user made that selection. In some embodiments, processor provides a third question asking the user why they selected the selected answer option in response to the second question and the answer options for the third question provide a plurality of different emotions or feelings to account for how the user feels about the selection that they made. For example, the answer options for the third question may comprise an array of user-selectable emotion image stimuli The interactive game component 132, 346 of the games module 124, 338 determines a trial outcome based on the answer(s) provided by the user in response to the one or more questions provided, at 3407. For example, the interactive game component 132, 346 of the games module 124, 338 compares the answers provided by the user with target answers associated with the questions to determine whether or not the answered provided by the user are deemed to be correct. In some embodiments, the interactive game component 132, 346 determines a trial outcome based on a percentage of correct answers provided by the user. In some embodiments, the trial outcome may further depend on a time value indicative of the time taken by the user to provide answers to the questions of the trial. Processor 102, 312 checks to see whether all trials are complete, at 3408. If more trials are to be performed, a new set of image stimuli is provided to the assessment platform and displayed to the user, at 3402. Once all trials are complete, the games module 124, 338 determines the game outcome based on the trial outcome(s), at 3309. The method moves to 3410, where the Multiple Morals game is considered to be complete. Processor 102, 312 may cause the Multiple Morals game to exit at step 3410.

The Multiple Morals game assesses moral reasoning across 1 or more trials, which may be 2, 3, 4, 5, 6, or more trials. In some embodiments, the Multiple Morals game may have 9 or 10 trials. The number of trials may be selected based on the age of the user. The trials are individualised based on the user's age and gender so that the stimuli closely resemble a real-life scenario for the user. The Multiple Morals game may cause three static photographs of a neutral social interaction to be shown on the screen. The user is then asked about their reaction to the photographs. For example, the user may be asked, “What would you do?” with multiple-choice options relevant to the scenario displayed on the screen. Alternatively or in addition, the user may be asked “How would you feel?” and told to select an emotion from a list that best describes how they would feel in that social interaction.

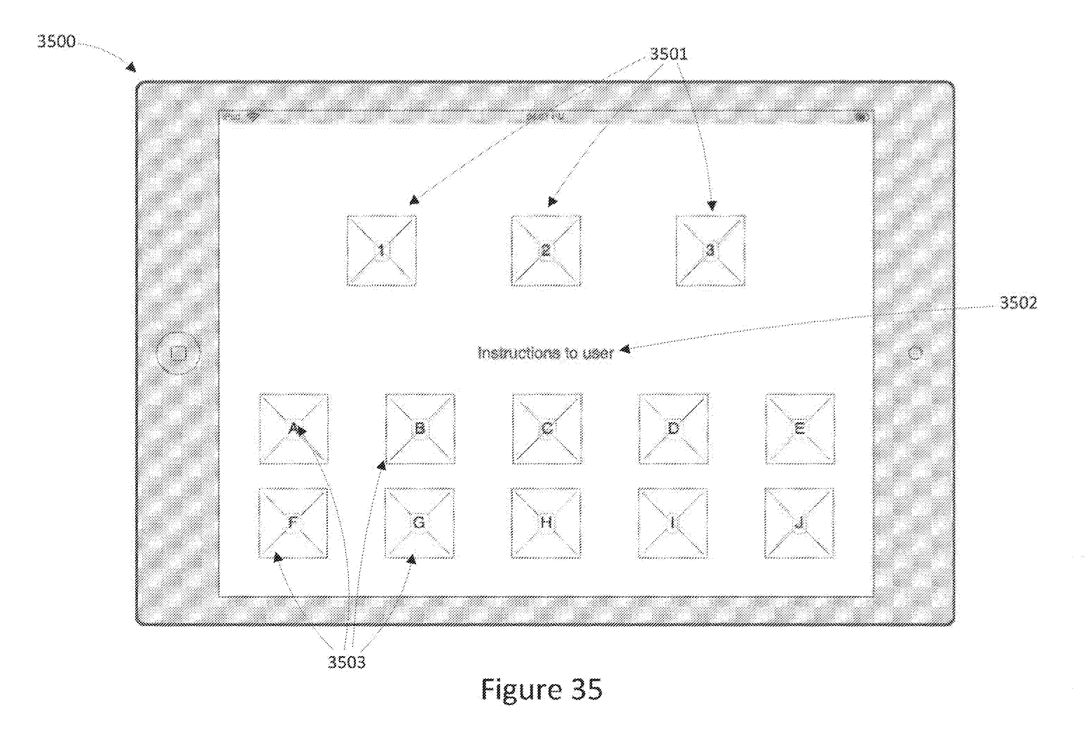

An example screen layout 3500 for the “Multiple Morals” game is shown in FIG. 35. Screen layout 3500 includes instructions photo stimuli 3501, instructions to the user 3502, and an array of emotion stimuli 3503. Instructions 3502 may prompt a user to select an emotion stimulus from array 3503 to describe how they felt about their decision in regard to the scenario shown in photo stimuli 3501. Photo stimuli 3501 may show a scene or scenario being played out, while emotion stimuli 3503 may show a number of cartoon faces showing various emotions.

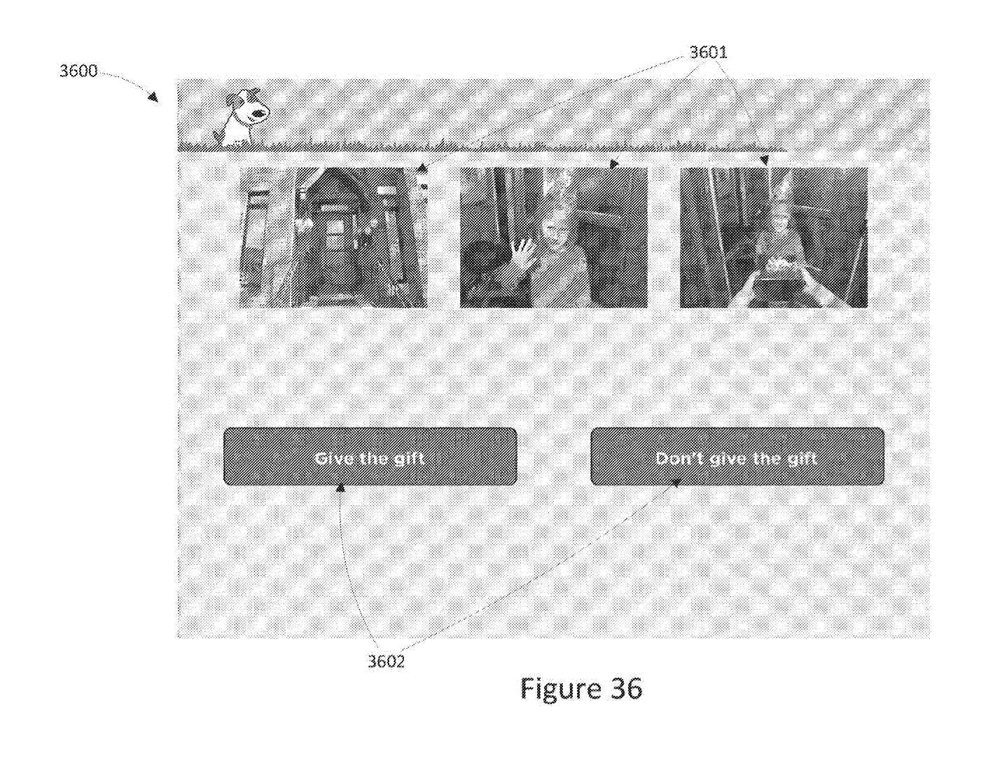

A first example screenshot 3600 for the “Multiple Morals” game is shown in FIG. 36. Screenshot 3600 includes photo stimuli 3601, and selectable options 3602. In the illustrated embodiments, photo stimuli 3601 depict the user arriving at a child's birthday party with a gift. Selectable options 3602 prompt the user to choose whether they give the gift, or don't give the gift.

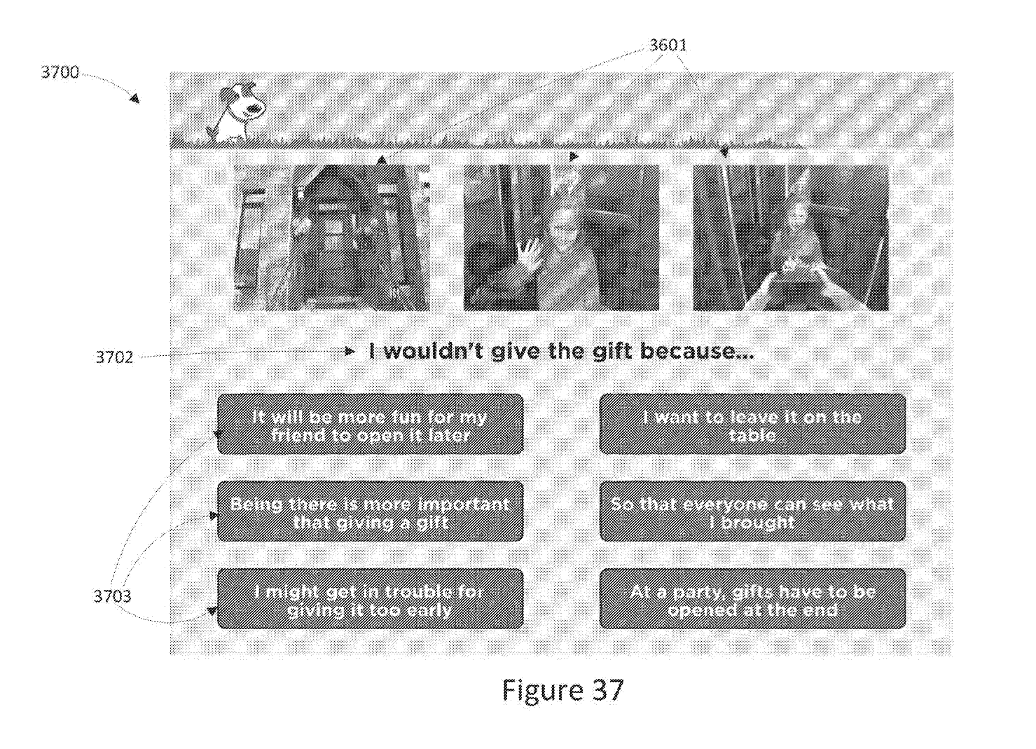

A second example screenshot 3700 for the “Multiple Morals” game is shown in FIG. 37, which may appear if the user was to press the “don't give the gift” option on screenshot 3600. Screenshot 3700 includes photo stimuli 3701, instructions 3702, and selectable options 3703. In the illustrated embodiment, instructions 3702 prompt a user to select an option in response to the phrase “I wouldn't give the gift because . . . ”. Selectable options 3703 include responses such as “I want to leave it on the table”, “I might get in trouble for giving it too early”, and “At a party, gifts have to be opened at the end”.

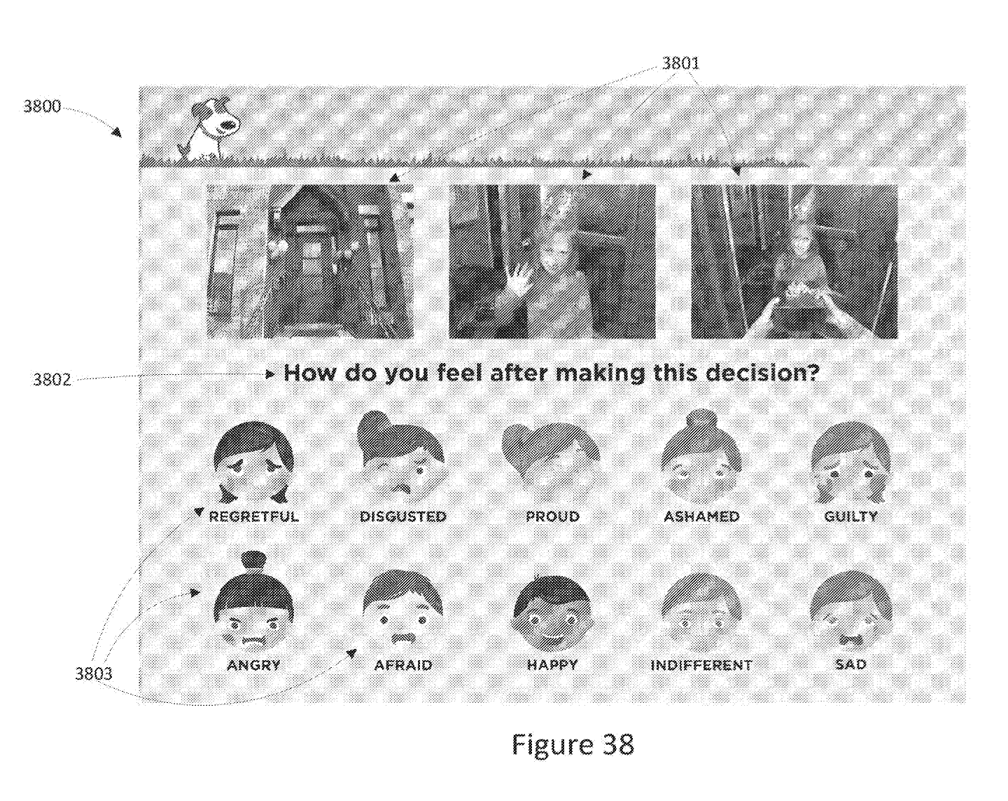

A third example screenshot 3800 for the “Multiple Morals” game is shown in FIG. 38. This screen may appear after the user makes a selection on screenshot 3700. Screenshot 3800 includes photo stimuli 3601, instructions 3802, and an array of emotion stimuli 3803. In the illustrated embodiments, instructions 3802 prompt a user to answer the question “how do you feel after making this decision?”. In the illustrated embodiment, the emotion stimuli 3803 are cartoon images of human faces showing a variety of emotions.

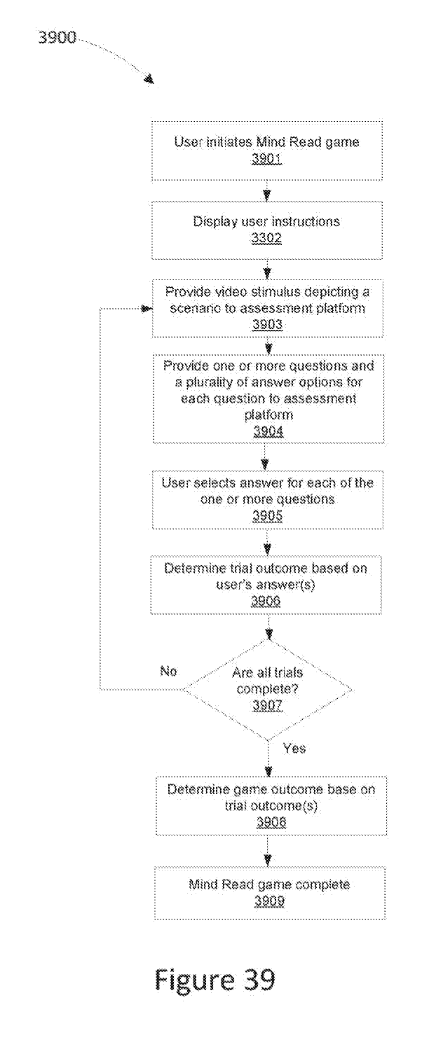

An example of a complex social cognition type game for determining a measure of theory of mind of the user is represented by FIG. 39. FIG. 39 is a process flow diagram of a computer-implemented method 3900 of determining a measure of theory of mind, the method 3900 operable by the assessment system 100, 302, and in particular, execution of the processor(s) 102, 312 of an interactive game component 132, 346 of the games module 124, 338 of the assessment application 118, 330. In this example, the complex social cognition type game is referred to as MIND READ—What's happening”.

Method 3900 starts at step 3901 when a user begins the “Mind Read—What's happening” game, at 3901, for example, by selecting selectable option 605 on the assessment platform. In some embodiments, processor 102, 312 executes an interactive game component 132, 346 that causes a set of instructions to be shown to the user on the assessment platform, at 3902, to direct the user to answer a question about a video that will be played. For example, the set of instructions may include an instruction such as “You are going to see some videos. Each one has a question. When you know the answer, touch YES or NO on the screen”. Processor 102, 312 causes a video stimulus to be provided to the assessment platform shown on a screen of the user interface 106, 324, at 3903. Processor 102, 312 causes one or more questions about the video stimulus and a set of user selectable answer options to each of the one or more questions to be provided to the assessment platform shown on the screen, at 3904. For example, in some embodiments, the question is “What's happening in the video?”. The user selects an answer to each of the one or more questions from the associated answer options provided using the user interface 106, 324, at 3905. The interactive game component 132, 346 of the games module 124, 338 determines a trial outcome based on the answer(s) provided by the user in response to the one or more questions provided, at 3906. For example, the interactive game component 132, 346 of the games module 124, 338 compares the answers provided by the user with target answers associated with the questions to determine whether or not the answered provided by the user are deemed to be correct. In some embodiments, the interactive game component 132, 346 determines a trial outcome based on a percentage of correct answers provided by the user. In some embodiments, the trial outcome may further depend on a time value indicative of the time taken by the user to provide answers to the questions of the trial. Processor 102, 312 determines or checks to see whether all trials are complete, at 3907. If more trials are to be performed, a new video stimulus is generated and provided to the assessment platform, at 3903. Once all trials are complete, the games module 124, 338 determines the game outcome based on the trial outcome(s), at 3908. The method then moves to 3909, where the Mind Read game is considered to be complete. Processor 102, 312 may cause the Mind Read game to exit at step 3909.

The Mind Read game assesses theory of mind across 1 or more trials, which may be 2, 3, 4, 5, 6, or more trials. In some embodiments, the Mind Read game may have 12 trials. The Mind Read game may cause a video of a vignette with a question relating to the video displayed at the top of the screen. Examples of questions include “Does he mean what he said?” and “Do they want to work together?” At the end of the video, the user may be asked to respond to the questions by selecting from a number of answers. In some embodiments, the available answers may be yes and no, for example

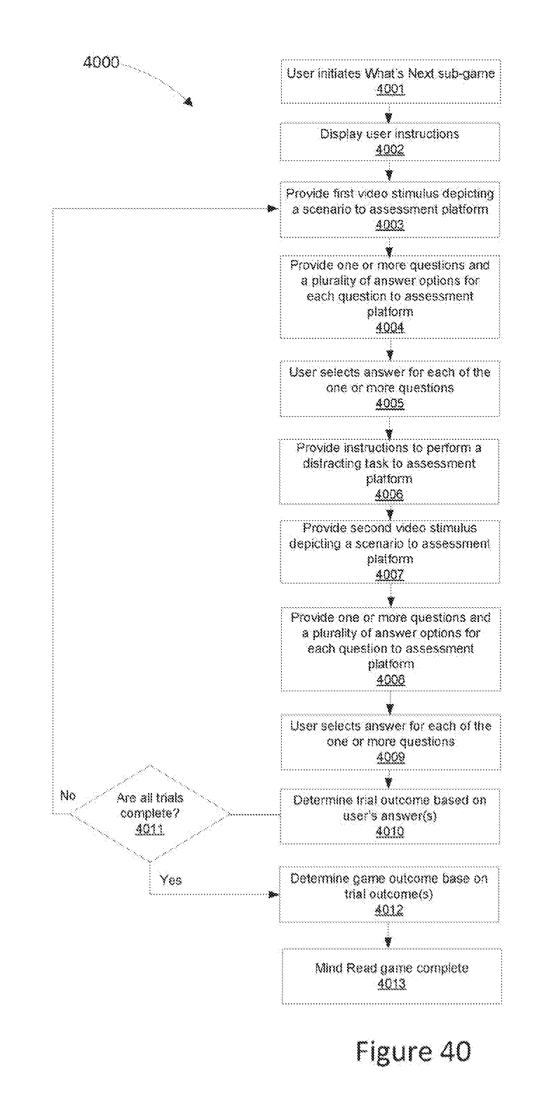

Another example of a complex social cognition type game for determining a measure of theory of mind of the user is represented by FIG. 40. FIG. 40 is a process flow diagram of a computer-implemented method 4000 of determining a measure of theory of mind, the method 4000 operable by the assessment system 100, 302, and in particular, execution of the processor(s) 102, 312 of an interactive game component 132, 346 of the games module 124, 338 of the assessment application 118, 330. In this example, the complex social cognition type game is referred to as MIND READ—What's Next”.

Method 4000 starts when a user initiates the “Mind Read—What's Next” game, at 4001, for example, by selecting selectable option 605 on the assessment platform. In some embodiments, processor 102, 312 executes an interactive game component 132, 346 that causes a set of instructions to be shown to the user on the assessment platform, at 4002 to direct the user to answer a set of questions about a set of videos that will be played and to perform distracting tasks. For example, the set of instructions may include an instruction such as “You are going to see some videos. Each one has a question. When you know the answer, touch YES or NO on the screen. Between some of the videos you will do things like count up to 100”. Processor 102, 312 causes a first video stimulus to be provided to the assessment platform shown on a screen of the user interface 106, 324, at 4003. Processor 102, 312 causes one or more questions about the video stimulus and a set of user selectable answer options to each of the one or more questions to be provided to the assessment platform shown on the screen, at 4004. For example, in some embodiments, the question is “What's happens next in the video?”. The user selects an answer to each of the one or more questions from the associated answer options provided using the user interface 106, 324, at 4005.

Processor 102, 312 causes a set of instructions to be provided to the assessment platform and shown to the user, instructing them to perform a distracting task, at 4006. For example, the set of instructions may include an instruction such as “Count to 10”. These distracting tasks are designed increase the memory demands of the task. Users may be given a time limit to complete the distracting task, which may be around 30 seconds or 1 minute in some embodiments. Users may perform the tasks vocally in some embodiments, and the user responses to the tasks may or may not be recorded.

Processor 102, 312 causes a second video stimulus to be to be provided to the assessment platform and shown on the screen of the user interface 106, 324, at 4007. Processor 102, 312 causes one or more questions about the second video stimulus and a set of user selectable answer options to each of the one or more questions to be provided to the assessment platform shown on the screen, at 4008. For example, in some embodiments, the question is “What's happens next in the video?”. The user selects an answer to each of the one or more questions from the associated answer options provided using the user interface 106, 324, at 4009. Processor 102, 312 determines or checks to see whether all trials are complete, at 4011. If more trials are to be performed, a new video stimulus is generated and provided to the assessment platform, at 4003. Once all trials are complete, the games module 124, 338 determines the game outcome based on the trial outcome(s), at 4012. The method moves to 4013, where the Mind Read—What's Next game is considered to be complete. Processor 102, 312 may cause the Mind Read—What's Next game to exit at step 4013.

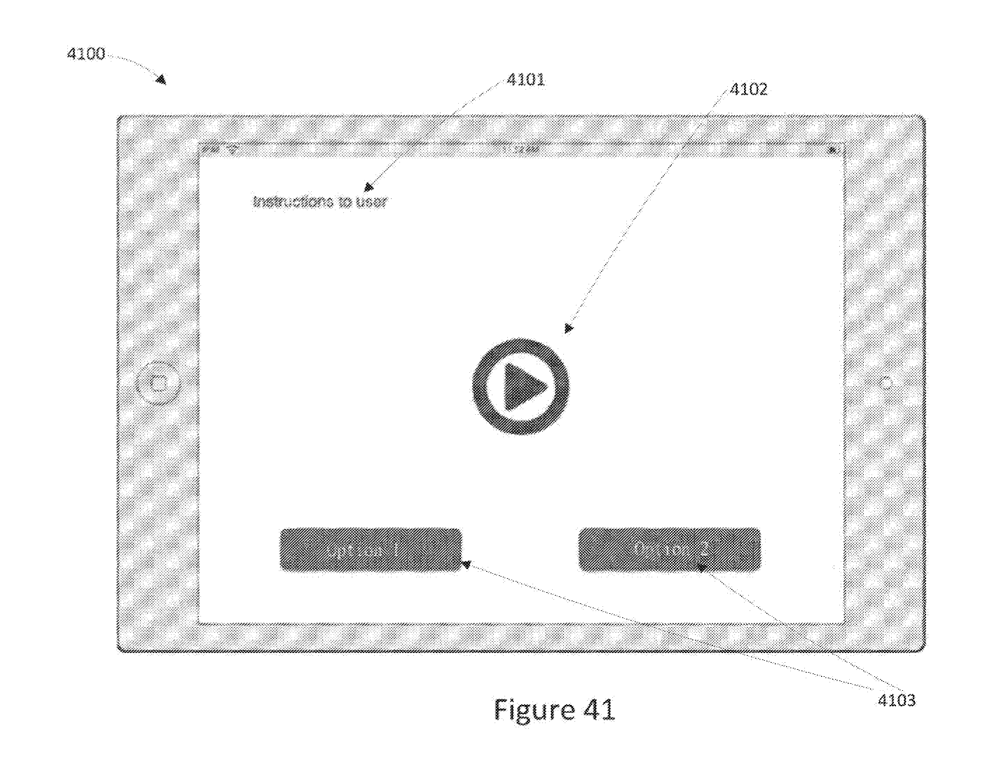

An example screen layout 4100 for the “Mind Read” game is shown in FIG. 41. Screen layout 4100 includes instructions 4101, a video player 4102, and a number of selectable options 4103 that display answers for the user to choose from. There may be two, three, four, five, six, or more selectable options 4103. Instructions 4101 may prompt a user to select an option from selectable options 4103 that answers a question displayed in instructions 4101.

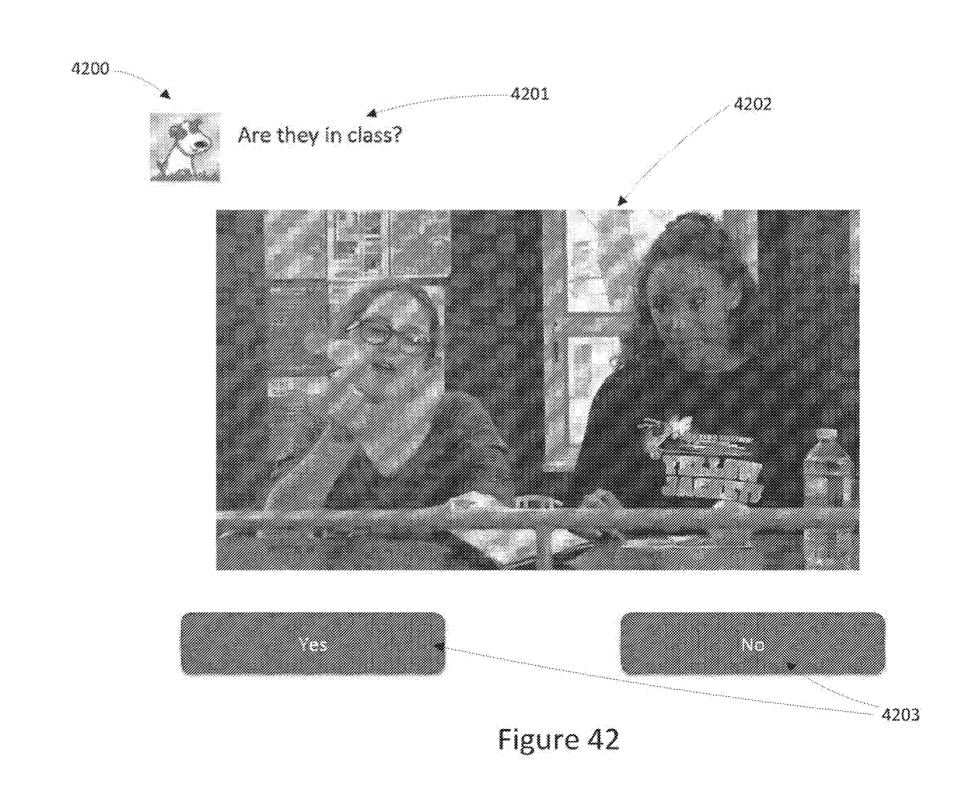

An example screenshot 4200 for the “Mind Read” game is shown in FIG. 42. Screenshot 4200 includes instructions 4201, a video still 4202, and a number of selectable options 4203 that display answers for the user to choose from. In the illustrated embodiment, instructions 4201 prompt a user to answer the question “Are they in class?”. In the illustrated embodiment, the selectable options 4203 available for the user to select from are “Yes” and “No”. Video still 4202 is showing a picture of two girls eating at a table in a cafeteria.

For each game or test performed, processor 102, 312 scores the user, which may include measures of accuracy and reaction time for each participant response. These scores are immediately available to examiners on task completion, in a user-friendly visual form. Scores are also fed into a local database to support calculation of summary scores for individual children and may be uploaded easily to an external database for research purposes.

An example of a method of administering a computer-based social skills questionnaire is represented by FIG. 43. FIG. 43 is a process flow diagram of a computer-implemented method 4300 of administering a computer-based social skills questionnaire, the method 4300 operable by the assessment system 100, 302. In this example, the computer-based social skills questionnaire is referred to as PEERS-Q.

Method 4300 starts at step 4301 when a user begins the PEERS-Q questionnaire Processor 102, 312 causes a set of instructions to be shown to the user at step 4302 to direct the user to answer a set of questions. For example, the set of instructions may include an instruction such as “Rate your child on the following items . . . ”. At step 4303, processor 102, 312 causes a new question to be shown on the screen, which may be displayed along with a number of selectable options for a user to select in answer to the question. At step 4304, the user selects an answer to the question. At step 4305, processor 102, 312 determines or checks to see whether all questions are complete. If more questions are to be answered, a new question is generated at step 4303. Once all questions have been answered, the method moves to step 4306, where the PEERS-Q questionnaire is considered to be complete. Processor 102, 312 may cause the PEERS-Q questionnaire to exit at step 4306.

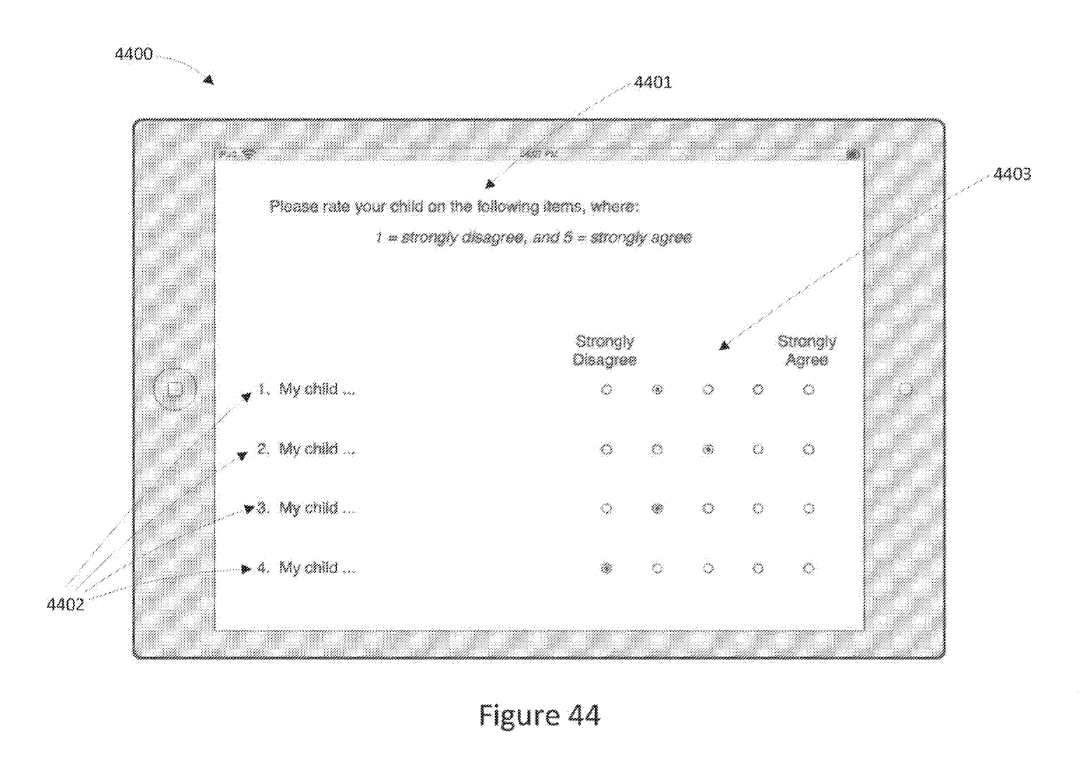

An example screenshot 4400 for the PEERS-Q questionnaire is shown in FIG. 44. Screenshot 4400 includes instructions 4401, an array of questions 4402, and an array of selectable options 4403. In the illustrated embodiment, instructions 4401 prompt a user to “Please rate your child on the following items, where 1=strongly disagree, and 5=strongly agree”. In the illustrated embodiment, four numbered questions 4402 are displayed, and array 4403 provides five selectable options for each question, the selectable options ranging from “Strongly Disagree” to “Strongly Agree”.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A diagnostic tool for assessing symptoms of neurodevelopmental conditions or disorders of patients, the diagnostic tool comprising:
a health data collection interface enabling a patent to interact with the diagnostic tool and to provide social feedback to stimulus provided by the diagnostic tool in in each of at least three test areas related to the patient's ability to intuit emotional state from physical cues, the three test areas corresponding to attention/executive function, communication, and social cognition, respectively;
an automated health data assessment device configured to obtain the social feedback of the patient for each test area of the at least three test areas, and to automatically assess social competency of the patient based at least partly on the social feedback and to generate an objective measurement of the social competency of the patient for use in diagnosing the patient for a potential neurodevelopmental condition.

2. An assessment system for assessing symptoms of neurodevelopmental conditions or disorders of patients, the assessment system comprising an assessment server configured to assess social competency of a user engaging with the assessment system, the assessment server arranged to communicate with the one or more computing devices across a communications network, the assessment server comprising:
at least one processor and memory comprising program code, which when executed by the at least one processor is configured to provide an assessment platform to a user interface of the one or more computing devices;
the memory comprising an assessment application including:
a games module; and
an assessment module;
wherein the games module includes at least one interactive game component in each of at least three social domains, each of the interactive game components, when executed by the at least one processor, being configured to provide an interactive game to the assessment platform and to determine an outcome for the interactive game based on user inputs received from a user at the computing device via the user interface;
wherein the assessment module, when executed by the at least one processor, is configured to assess social competency of the user of the assessment platform based on the outcome of at least one interactive game in each of the at least three social domains and to generate an assessment outcome; and
wherein the at least three social domains comprise attention/executive function, communication, and social cognition.

3. The assessment system of claim 1 or claim 2, wherein the at least one of the interactive game components in the attention/executive function social domain comprises an attention/executive function game component configured to provide an attention/executive function game and wherein the outcome of the attention/executive function game provides a measure of attention/executive function of the user.

4. The assessment system of claim 3, wherein the attention/executive function game is configured to determine a measure of at least one of processing speed, attention, self-regulation and flexibility.

5. The assessment system of any one of claims 1 to 4, wherein the at least one of the interactive game components in the communication social domain comprises a communication game component configured to provide a communication game and wherein the outcome of the communication game provides a measure of communication ability of the user.

6. The assessment system of claim 5, wherein the communication game is configured to determine a measure of at least one of pragmatic language, prosody, and non-verbal language comprehension of the user.

7. The assessment system of any one of claims 1 to 6, wherein the at least one of the interactive game components in the social cognition social domain comprises a social cognition game component configured to provide a social cognition game and wherein the outcome of the social cognition game provides a measure of social cognition of the user.

8. The assessment system of claim 7, wherein the social cognition game is configured to determine a measure of at least one of emotional perception, intent attribution, empathy, perspective taking, theory of mind and moral reasoning.

9. The assessment system of any one of the preceding claims, wherein the memory further comprises a weighting module, which when executed by the at least one processor, is configured to apply a weighting to the outcome of the at least one interactive game in each of the at least three social domains and wherein the assessment outcome is based on the weighted game outcomes.

10. The assessment system of any one of the preceding claims, wherein the assessment outcome comprises a social quotient score indicative of social competency of the user.

11. The assessment system of any preceding claim, wherein the assessment module, when executed by the at least one processor, is configured to provide the assessment outcome to the assessment platform.

12. The assessment system of any one of the preceding claims, wherein the memory further comprises a records module, which when executed by the at least one processor, is configured to create, store, modify and delete user records associated with users of the assessment platform.

13. The assessment system of claim 12, wherein user records comprise information derived from answers to social skills questionnaires provided to the user and/or people associated with the user.

14. The assessment system of claim 12, wherein a user's record of the user records comprise at least one of game outcomes, weighted game outcomes and assessment outcomes associated with the user.

15. The assessment system of any one of claims 12 to 14, wherein the assessment outcome is further based on data stored in the user records.

16. The assessment system of any one of the preceding claims, wherein the assessment outcome is based on normative data comprising age and gender norms for the diagnosis of social impairment.

17. The assessment system of claim 16, wherein the assessment module, when executed by the at least one processor, is configured to assess social competency of the user by comparing the outcome of the at least one interactive game with the normative data, wherein the normative data is retrieved from a remote server.

18. The assessment system of claim 16 when directly or indirectly dependent on claim 1, wherein the assessment system comprises a network interface configured to communicate with a remote server over a communications network and wherein the assessment module is configured to assess social competency of the user by causing the assessment system to transmit information associated with the user, including the outcome of the at least one interactive game, to a remote server via the network interface and to receive from the remote server via the network interface an assessment of the social competency of the user, and wherein the assessment module is configured to generate the assessment outcome based on the received assessment of the social competency.

19. The assessment system of claim 1 or claims 3 to 18 when directly or indirectly dependent on claim 1, wherein the assessment system comprises an assessment server and a computing device in communication with the assessment server across a communications network, wherein the assessment server comprises the at least one processor and memory and the device comprises the user interface.

20. The assessment system of any one of the preceding claims, wherein the assessment module, when executed by the at least one processor, is further configured to provide a computer-based social skills questionnaire and to receive responses to the questionnaire, wherein the responses to the questionnaire are associated with a user and are used to assess a user social competency of the user.

21. The assessment system of any one of the preceding claims, wherein the assessment module, when executed by the at least one processor, is further configured to determine a treatment plan based on the generated assessment outcome.

22. A computer implemented method for assessing social competency of a user of an assessment system, the method comprising:
providing, by an assessment application of the assessment system, an assessment platform;
providing, by a games module of the assessment application, at least one interactive game in each of at least three social domains to the assessment platform;
allowing the user of the assessment system to play the at least one interactive game in each of the at least three social domains using a user interface of the assessment system;
determining, by the games module, an outcome for each of the interactive games played by the user based on user inputs received from the user of the assessment system via the user interface;
assessing, by an assessment module of the assessment application, a social competency of the user of the assessment platform based on the outcome of the at least one interactive game in each of the at least three social domains; and
generating, by the assessment module, an assessment outcome indicative of the social competency of the user;
wherein the at least three social domains comprise attention/executive function, communication, and social cognition.

23. The computer implemented method of claim 22, wherein the at least one of the interactive games in the attention/executive function social domain comprises an attention/executive function game and wherein the outcome of the attention/executive function game provides a measure of attention/executive function of the user.

24. The computer implemented method of claim 23, wherein the attention/executive function game is configured to determine a measure of at least one of processing speed, attention, self-regulation and flexibility.

25. The computer implemented method of any one of claims 22 to 24, wherein the at least one of the interactive games in the communication social domain comprises a communication game and wherein the outcome of the communication game provides a measure of communication ability of the user.

26. The computer implemented method of claim 25, wherein the communication game is configured to determine a measure of at least one of pragmatic language, prosody, and non-verbal language comprehension of the user.

27. The computer implemented method of any one of claims 22 to 26, wherein the at least one of the interactive games in the social cognition social domain comprises a social cognition game and wherein the outcome of the social cognition game provides a measure of social cognition of the user.

28. The computer implemented method of claim 27, wherein the social cognition game is configured to determine a measure of at least one of emotional perception, intent attribution, empathy, perspective taking, theory of mind and moral reasoning.

29. The computer implemented method of any one of claims 22 to 28, further comprising, applying by a weighting module of the assessment application, a weighting to the outcome of the at least one interactive game in each of the at least three social domains and wherein the assessment outcome is based on the weighted game outcomes.

30. The computer implemented method of any one of claims 22 to 29, wherein the assessment outcome comprises a social quotient score indicative of social competency of the user.

31. The computer implemented method of any one of claims 22 to 29, further comprising providing, by the assessment module, the assessment outcome to the assessment platform.

32. The computer implemented method of any one of claims 22 to 30, further comprising maintaining by a records module of the assessment application, a user record associated with users of the assessment platform.

33. The computer implemented method of claim 32, wherein user records comprise information derived from answers to social skills questionnaires provided to the user and/or people associated with the user.

34. The computer implemented method of claim 33, wherein a user's record of the user records comprise at least one of game outcomes, weighted game outcomes and assessment outcomes associated with the user.

35. The computer implemented method of any one of claims 22 to 34, further comprising determining, by the assessment module, the assessment outcome based on data stored in the user records.

36. The computer implemented method of any one of claims 22 to 35, further comprising determining, by the assessment module, the assessment outcome based on normative data comprising age and gender norms for the diagnosis of social impairment.

37. The computer implemented method of claim 36, further comprising assessing, by the assessment module, a social competency of the user by comparing the outcome of the at least one interactive game with the normative data, wherein the normative data is retrieved from a remote server.

38. The computer implemented method of any one of claims 22 to 37, further comprising providing, by the assessment application, a computer-based social skills questionnaire and receiving, by the assessment application, responses to the questionnaire, wherein the responses to the questionnaire are associated with a user and are used to assess a user social competency of the user.

39. The computer implemented method of any one of claims 22 to 38, further comprising determining, by the assessment application, a treatment plan based on the generated assessment outcome.

40. A computer implemented method for determining a measure of selective attention of a user of an assessment system, the method comprising:
performing, by a games module of an assessment application of the assessment system, a plurality of rounds of a selective attention game, wherein each round comprises:
providing, by an interactive game component of the games module, a target image and an array of images to an assessment platform provided on a user interface of the assessment system, wherein the array of images also comprises the target image;
receiving, by the interactive game component, an indication of a selected image of the array of images from the user via the user interface;
comparing, by the interactive game component, the selected image with the target image; and
determining, by the interactive game component, a round outcome based on the comparison; and
determining, by the games module, a game outcome based on the round outcomes of the plurality of games, wherein the game outcome is indicative of a measure of selective attention of the user.

41. A computer implemented method for determining a measure of reaction time of a user of an assessment system, the method comprising:
performing, by a games module of an assessment application of the assessment system, a plurality of rounds of a reaction time game, wherein each round comprises:
providing, by an interactive game component of the games module, an object to an assessment platform provided on a user interface of the assessment system, wherein the object is positioned at one of a plurality of locations on the assessment platform;
receiving, by the interactive game component, an indication of a selected location on the assessment platform from the user via the user interface;
determining by the interactive game component, a time value for a length of time taken by the user to provide indication of the selected location;
comparing, by the interactive game component, the selected location with position of the object; and
determining, by the interactive game component, a round outcome based on the time value and the comparison; and
determining, by the games module, a game outcome based on the round outcome of the plurality of rounds, wherein the game outcome is indicative of a measure of reaction time of the user.

42. A computer implemented method for determining a measure of information processing of a user of an assessment system, the method comprising:
performing, by a games module of an assessment application of the assessment system, one or more rounds of an information processing game, wherein each round comprises:
providing, by an interactive game component of the games module, an array of images depicting a plurality of emotions to an assessment platform provided on a user interface of the assessment system, wherein each image of the array depicts one of the plurality of emotions;
for each of the images of the array, receiving, by the interactive game component, an indication of a selected one of the images of the array of images and determining that an identified emotion provided by the user corresponds substantially with the emotion depicted by the selected image;
determining, by the interactive game component, a time value for the time taken by the user to correctly identify emotions depicted by all of the images of the array; and
determining, by the interactive game component, a round outcome based on the time value; and
determining, by the games module, a game outcome based on the round outcomes of the plurality of games, wherein the game outcome is indicative of a measure of measure of information processing of the user.

43. The computer implemented method of claim 42, wherein determining that an identified emotion provided by the user corresponds substantially with the emotion depicted by the selected image comprises, for each of the images of the array, receiving, by the games module, an input from the user via the user interface, wherein the input is indicative of an identified emotion and determining, by the games module, that the identified emotion corresponds substantially with a target emotion associated with the selected image by comparing the identified emotion to the target emotion.

44. The computer implemented method of claim 42, wherein determining that an identified emotion provided by the user corresponds substantially with the emotion depicted by the selected image comprises, for each of the images of the array, receiving, by the games module, an input from an advisor assisting the user indicating that identified emotion corresponds substantially with the emotion depicted by the selected image.

45. A computer implemented method for determining a measure of non-verbal language comprehension of a user of an assessment system, the method comprising:
performing, by a games module of an assessment application of the assessment system, one or more rounds of an non-verbal language comprehension game, wherein each round comprises:
providing, by an interactive game component of the games module, an array of images to an assessment platform provided on a user interface of the assessment system, wherein the array of images comprises a first image depicting a first emotion and each of the remaining images depicting a second emotion and the first emotion is different from the second emotion;
receiving, by the interactive game component, an indication of a selected image from the array of image stimuli displayed on the assessment platform from the user via the user interface;
comparing, by the interactive game component, the selected image to the first image; and
determining, by the interactive game component, a round outcome based on the comparison; and
determining, by the games module, a game outcome based on the round outcome of the one or more rounds, wherein the game outcome is indicative of a measure non-verbal language comprehension of the user.

46. A computer implemented method for determining a measure of social intent comprehension of a user of an assessment system, the method comprising:
performing, by a games module of an assessment application of the assessment system, one or more rounds of a social intent comprehension game, wherein each round comprises:
providing, by an interactive game component of the games module, an array of images to an assessment platform provided on a user interface of the assessment system, wherein the array of images comprises a first image depicting a first social interaction and each of the remaining images depicting a second social interaction and the first social interaction is different from the second social interaction;
receiving, by the interactive game component, a selected image from the array of images displayed on the assessment platform from the user via the user interface;
comparing, by the interactive game component, the selected image to the first image; and
determining, by the interactive game component, a round outcome based on the comparison; and
determining, by the games module, a game outcome based on the round outcome of the one or more rounds, wherein the game outcome is indicative of a measure of social intent comprehension of the user.

47. A computer implemented method for determining a measure of pragmatics of a user of an assessment system, the method comprising:
performing, by a games module of an assessment application of the assessment system, one or more rounds of a pragmatics game, wherein each round comprises:
providing, by an interactive game component of the games module, a set of images to an assessment platform provided on a user interface of the assessment system, wherein the set of images, when placed in order, collectively depict a scenario;
receiving, by the interactive game component, an ordered set of the set of images from the user via the user interface;
comparing, by the interactive game component, the ordered set of the images with a target ordered set of the images; and
determining, by the interactive game component, a round outcome based on the comparison; and
determining, by the games module, a game outcome based on the round outcome of the one or more plurality of rounds, wherein the game outcome is indicative of a measure pragmatics of the user.

48. A computer implemented method for determining a measure of prosody of a user of an assessment system, the method comprising:
performing, by a games module of an assessment application of the assessment system, one or more rounds of a prosody game, wherein each round comprises:
providing, by an interactive game component of the games module, a set of images to an assessment platform provided on a user interface of the assessment system, wherein each image of the set of images depicts a different emotion;
providing, by the interactive game component, an audio snippet to the assessment platform provided on a user interface of the assessment system, wherein the audio snippet suggests an emotion;
receiving, by the interactive game component, an indication of a selected image of the set of images from the user via the user interface;
comparing, by the interactive game component, the selected image with a target image associated with the audio snippet; and
determining, by the interactive game component, a round outcome based on the comparison; and
determining, by the games module, a game outcome based on the round outcome of the one or more plurality of rounds, wherein the game outcome is indicative of a measure of prosody of the user.

49. A computer implemented method for determining a measure of emotion perception of a user of an assessment system, the method comprising:
performing, by a games module of an assessment application of the assessment system, one or more rounds of an emotion perception game, wherein each round comprises:
providing, by an interactive game component of the games module, a set of images to an assessment platform provided on a user interface of the assessment system, wherein each image of the set of images depicts a different emotion;
providing, by the interactive game component, an emotion label to the assessment platform provided on the user interface of the assessment system;
receiving, by the interactive game component, an indication of a selected image of the array of images from the user via the user interface;
determining, by the interactive game component, that the selected image is associated with the emotion label in response to determining that the emotion label corresponds substantially with a target label associated with the selected image;
determining, by the interactive game component, a time value for the length of time taken by the user to select the image associated with the emotion label; and
determining, by the interactive game component, a round outcome based on the time value; and
determining, by the games module, a game outcome based on the round outcome of the one or more plurality of rounds, wherein the game outcome is indicative of a measure of emotion perception of the user.

50. A computer implemented method for determining a measure of attribution of social intent of a user of an assessment system, the method comprising:
performing, by a games module of an assessment application of the assessment system, one or more rounds of a social intent game, wherein each round comprises:
providing, by an interactive game component of the games module, a video stimulus to the assessment platform provided on a user interface of the assessment system, wherein the video stimulus suggests an emotion;
providing, by the interactive game component, a set of images to an assessment platform provided on a user interface of the assessment system, wherein each image of the set of images depicts a different emotion;
providing, by the interactive game component, one or more questions relating to the social intent of the video stimulus and a set of answer options associated with each of the one or more questions;
receiving, by the interactive game component, an indication of a selected image of the set of images from the user via the user interface;
receiving, by the interactive game component, a selected answer to each of the one or more questions from the user via the user interface;
comparing, by the interactive game component, the selected image with a target image associated with the video stimulus and the selected answer with a target answer for each of the one or more questions; and
determining a round outcome based on the comparison; and
determining, by the games module, a game outcome based on the round outcome of the one or more plurality of rounds, wherein the game outcome is indicative of a measure of measure of attribution of social intent of the user.

51. A computer implemented method for determining a measure of moral reasoning of a user of an assessment system, the method comprising:
performing, by a games module of an assessment application of the assessment system, one or more rounds of a moral reasoning game, wherein each round comprises:
providing, by an interactive game component of the games module, one or more images to the assessment platform provided on a user interface of the assessment system, wherein each of the one or more images depicts a problematic situation;
providing, by the interactive game component, one or more questions relating to the problematic situation depicted in the one or more images and a set of answer options associated with each of the one or more questions;
receiving, by the games module, a selected answer to each of the one or more questions from the user via the user interface;
comparing, by the games module, the selected answer with a target answer associated with each of the one or more questions; and
determining a round outcome based on the comparison; and
determining, by the games module, a game outcome based on the round outcome of the one or more plurality of rounds, wherein the game outcome is indicative of a measure of moral reasoning of the user.

52. A computer implemented method for determining a measure of theory of mind of a user of an assessment system, the method comprising:
performing, by a games module of an assessment application of the assessment system, one or more rounds of a theory of mind game, wherein each round comprises:
providing, by an interactive game component of the games module, a video stimulus to the assessment platform provided on a user interface of the assessment system, wherein the video stimulus depicts a situation;
providing, by the interactive game component, one or more questions relating to the situation depicted in the video stimulus and a set of answer options associated with each of the one or more questions;
receiving, by the games module, a selected answer to each of the one or more questions from the user via the user interface;
comparing, by the games module, the selected answer with a target answer associated with each of the one or more questions; and
determining a round outcome based on the comparison; and
determining, by the games module, a game outcome based on the round outcome of the one or more plurality of rounds; wherein the game outcome is indicative of a measure of theory of mind of the user.

53. A computer implemented method for determining a measure of theory of mind of a user of an assessment system, the method comprising:
performing, by a games module of an assessment application of the assessment system, one or more rounds of a theory of mind game, wherein each round comprises:
providing, by an interactive game component of the games module, a first video stimulus to the assessment platform provided on a user interface of the assessment system, wherein the first video stimulus depicts a first situation;
providing, by the interactive game component, one or more questions relating to the first situation depicted in the video stimulus and a set of answer options associated with each of the one or more questions;
receiving, by the games module, a selected answer to each of the one or more questions associated with the first video stimulus from the user via the user interface;
providing, by the interactive game component, a second video stimulus to the assessment platform provided on a user interface of the assessment system, wherein the second video stimulus depicts a second situation different from that depicted in the first video stimulus;
providing, by the interactive game component, one or more questions relating to the second situation depicted in the video stimulus and a set of answer options associated with each of the one or more questions;
receiving, by the games module, a selected answer to each of the one or more questions associated with the second video stimulus from the user via the user interface;
comparing, by the games module, the selected answers with target answers associated with each of the one or more questions; and
determining a round outcome based on the comparison; and
determining, by the games module, a game outcome based on the round outcome of the one or more plurality of rounds, wherein the game outcome is indicative of a measure of theory of mind of the user.

54. An assessment system for assessing social competency of a user of the assessment system, the assessment system comprising:
at least one processor, a user interface and memory comprising program code, which when executed by the at least one processor is configured to provide an assessment platform to the user interface;
the memory comprising an assessment application including:
a games module; and
an assessment module;
wherein the games module is configured to perform the computer implemented method of any one of claims 38 to 53.

55. An assessment system comprising an assessment server configured to assess social competency of a user engaging with the assessment system, the assessment server arranged to communicate with the one or more computing devices across a communications network, the assessment server comprising:
at least one processor and memory comprising program code, which when executed by the at least one processor is configured to provide an assessment platform to a user interface of the one or more computing devices;
the memory comprising an assessment application including:
a games module; and
an assessment module;
wherein the games module is configured to perform the computer implemented method of any one of claims 38 to 53.

56. The assessment system of claim 54 or claim 55, wherein the games module includes at least one interactive game component in each of at least three social domains, each of the interactive game components, when executed by the at least one processor, being configured to provide an interactive game to the assessment platform and to determine an outcome for the interactive game based on user inputs received from the user of the assessment system via the user interface;
wherein the assessment module, when executed by the at least one processor, is configured to assess social competency of the user of the assessment platform based on the outcome of at least one interactive game in each of the at least three social domains and to generate an assessment outcome; and
wherein the at least three social domains comprise attention/executive function, communication, and social cognition.

57. A computer program product comprising a computer readable medium encoded with computer executable instructions, which when executed in a computer system, is effective to cause the computer system to carry out the method of any one of claims 22 to 53.

58. The computer readable medium of claim 57, wherein the computer readable medium is a non-transitory computer readable medium.
